(19) United States
(12) Patent Application Publication
(10) Pub. No.: US 2007/0264193 A1
(43) Pub. Date: Nov. 15, 2007

(54) DIAGNOSTICS AND TREATMENTS FOR TUMORS

(75) Inventors: Farbod Shojaei, Santa Clara, CA (US); Cuiling Zhong, Palo Alto, CA (US); Megan Baldwin, San Francisco, CA (US); Hans-Peter Gerber, Bellevue, WA (US); Napoleone Ferrara, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/692,681

(22) Filed: Mar. 28, 2007

Related U.S. Application Data
(60) Provisional application No. 60/787,720, filed on Mar. 29, 2006.

(51) Int. Cl. A61K 39/395; A61B 5/00; C12Q 1/06

(52) U.S. Cl. 424/9.1; 424/158.1; 435/39

(57) ABSTRACT

Methods for the treatment of cancer with combination therapies that include anti-VEGF antibodies are provided. Methods for diagnosing resistant tumors are also provided.

### RELATED APPLICATIONS

This application claims benefit from U.S. Provisional Application No. 60/787,720, filed Mar. 29, 2006.

### FIELD OF THE INVENTION

The invention relates to the field of tumor growth and tumor type. The invention relates to inhibitors and diagnostics markers for tumors, and uses of such for the diagnosis and treatment of cancer and tumor growth.

### BACKGROUND

Malignant tumors (cancers) are a leading cause of death in the United States, after heart disease (see, e.g., Boring et al., CA Cancel J. Clin. 43:7(1993)). Cancer is characterized by the increase in the number of abnormal, or neoplastic, cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites via a process called metastasis. In a cancerous state, a cell proliferates under conditions in which normal cells would not grow. Cancer manifests itself in a wide variety of forms, characterized by different degrees of invasiveness and aggressiveness.

Various types of therapies have been used to treat cancer. For example, surgical methods are used to remove cancerous or dead tissue. Radiotherapy, which works by shrinking solid tumors, and chemotherapy, which kills rapidly dividing cells, are used as cancer therapies. In addition, anti-angiogenesis agents are an effective anticancer strategy. These therapies are also being enhanced, while other therapies are being developed, e.g., immunotherapies.

It is now well established that angiogenesis is implicated in the pathogenesis of a variety of disorders. These include solid tumors and metastasis, atherosclerosis, retrolental fibroplasia, hemangiomas, chronic inflammation, intraocular neovascular diseases such as proliferative retinopathies, e.g., diabetic retinopathy, age-related macular degeneration (AMD), neovascular glaucoma, immune rejection of transplanted corneal tissue and other tissues, rheumatoid arthritis, and psoriasis. Folkman et al., J. Biol. Chem., 267:10931-10934 (1992); Klagsbrun et al., Annu. Rev. Physiol. 53:217-239 (1991); and Garner A., “Vascular diseases”, In: Pathobiology of Ocular Disease. A Dynamic Approach, Garner A., Klintworth G K, eds., 2nd Edition (Marcel Dekker, NY, 1994), pp 1625-1710.

In the case of tumor growth, angiogenesis appears to be crucial for the transition from hyperplasia to neoplasia, and for providing nourishment for the growth and metastasis of the tumor. Folkman et al., Nature 339:58 (1989). Neovascularization allows the tumor cells to acquire a growth advantage and proliferative autonomy compared to the normal cells. A tumor usually begins as a single aberrant cell which can proliferate only to a size of a few cubic millimeters due to the distance from available capillary beds, and it can stay ‘dormant’ without further growth and dissemination for a long period of time. Some tumor cells then switch to the angiogenic phenotype to activate endothelial cells, which proliferate and mature into new capillary blood vessels. These newly formed blood vessels not only allow for continued growth of the primary tumor, but also for the dissemination and recolonization of metastatic tumor cells. Accordingly, a correlation has been observed between density of microvessels in tumor sections and patient survival in breast cancer as well as in several other tumors. Weidner et al., N. Engl. J. Med 324:1-6 (1991); Horak et al., Lancet 340:1120-1124 (1992); Macchiarini et al., Lancet 340:145-146 (1992). The precise mechanisms that control the angiogenic switch is not well understood, but it is believed that neovascularization of tumor mass results from the net balance of a multitude of angiogenesis stimulators and inhibitors (Folkman, 1995, Nat Med 1(1):27-31).

Recognition of vascular endothelial growth factor (VEGF) as a primary regulator of angiogenesis in pathological conditions has led to numerous attempts to block VEGF activities. VEGF is one of the best characterized and most potent positive regulators of angiogenesis. See, e.g., Ferrara, N. & Kerbel, R. S. Angiogenesis as a therapeutic target. Nature 438:967-74 (2005). In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx. Ferrara and Davis-Smyth (1997) Endocrine Rev. 18:4-25. Moreover, studies have reported mitogenic effects of VEGF on a few non-endothelial cell types, such as retinal pigment epithelial cells, pancreatic duct cells and Schwann cells. See, e.g., Guerrin et al. J. Cell Physiol. 164:385-394 (1995); Oberg-Welsh et al. Mol. Cell. Endocrinol. 126:125-132 (1997); and, Sondell et al. J. Neurosci. 19:5731-5740 (1999).

There has been numerous attempts to block VEGF activities. Inhibitory anti-VEGF receptor antibodies, soluble receptor constructs, antisense strategies, RNA aptamers against VEGF and low molecular weight VEGF receptor tyrosine kinase (RTK) inhibitors have all been proposed for use in interfering with VEGF signaling. See, e.g., Siemeister et al. Cancer Metastasis Rev. 17:241-248 (1998). Anti-VEGF neutralizing antibodies have been shown to suppress the growth of a variety of human tumor cell lines in nude mice (Kim et al. Nature 362:841-844 (1993); Warren et al. J. Clin. Invest. 95:1789-1797 (1995); Borgström et al. Cancer Res. 56:4032-4039 (1996); and Melnyk et al. Cancer Res. 56:921-924 (1996)) and also inhibit intraocular angiogenesis in models of ischemic retinal disorders (Adamis et al. Arch. Ophthalmol. 114:66-71 (1996)). Indeed, a humanized anti-VEGF antibody, bevacizumab (AVASTIN®, Genentech, South San Francisco, Calif.) has been approved by the US FDA as a first-line therapy for metastic colorectal cancer. See, e.g., Ferrara et al., Nature Reviews Drug Discovery, 3:391-400 (2004).

However, the long-term ability of therapeutic compounds to interfere with tumor growth is frequently limited by the development of drug resistance. Several mechanisms of resistance to various cytotoxic compounds have been identified and functionally characterized, primarily in unicellular tumor models. See, e.g., Longley, D. B. & Johnston, P. G. Molecular mechanisms of drug resistance. J Pathol 205:275-92 (2005). In addition, host stromal-tumor cell interactions may be involved in drug-resistant phenotypes. Stromal cells secrete a variety of pro-angiogenic factors and are not prone to the same genetic instability and increases in mutation rate as tumor cells (Kerbel, R. S. Inhibition of tumor angiogenesis as a strategy to circumvent acquired resistance to anti-cancer therapeutic agents. Bioessays 13:31-6 (1991). Reviewed by Ferrara & Kerbel and Hazlehurst et al. in Ferrara, N. & Kerbel, R. S. Angiogenesis as a therapeutic target. Nature 438:967-74 (2005); and, Hazlehurst, L. A., Landowski, T. H. & Dalton, W. S. Role of the tumor microenvironment in mediating de novo resistance to drugs and physiological mediators of cell death. Oncogene 22:7396-402 (2003).

In preclinical models, VEGF signaling blockade with the humanized monoclonal antibody bevacizumab (AVASTIN®, Genentech, South San Francisco, Calif.) or the murine precursor to bevacizumab (A4.6.1 (hybridoma cell line producing A4.6.1 deposited on Mar. 29, 1991, ATCC HB-10709)) significantly inhibited tumor growth and reduced tumor angiogenesis in most xenograft models tested (reviewed by Gerber & Ferrara in Gerber, H. P. & Ferrara, N. Pharmacology and pharmacodynamics of bevacizumab as monotherapy or in combination with cytotoxic therapy in preclinical studies. Cancer Res 65:671-80 (2005)). The pharmacologic effects of single-agent anti-VEGF treatment were most pronounced when treatment was started in the early stages of tumor growth. If treatment was delayed until tumors were well established, the inhibitory effects were typically transient, and tumors eventually developed resistance. See, e.g., Klement, G. et al. Differences in therapeutic indexes of combination metronomic chemotherapy and an anti-VEGFR-2 antibody in multidrug-resistant human breast cancer xenografts. Clin Cancer Res 8:221-32 (2002). The cellular and molecular events underlying such resistance to anti-VEGF treatment are complex. See, e.g., Casanovas, O., Hicklin, D. J., Bergers, G. & Hanahan, D. Drug resistance by evasion of antiangiogenic targeting of VEGF signaling in late-stage pancreatic islet tumors. Cancer Cell 8:299-309 (2005); and, Kerbel, R. S. et al. Possible mechanisms of acquired resistance to anti-angiogenic drugs: implications for the use of combination therapy approaches. Cancer Metastasis Rev 20:79-86 (2001). A variety of factors may be involved. For example, combination treatment with compounds targeting VEGF and fibroblast growth factor (FGF) signaling improved efficacy and delayed onset of resistance in late-stage tumors in a genetic model of pancreatic islet carcinogenesis. See, Casanovas, O., Hicklin, D. J., Bergers, G. & Hanahan, D. Drug resistance by evasion of antiangiogenic targeting of VEGF signaling in late-stage pancreatic islet tumors. Cancer Cell 8, 299-309 (2005). Other investigators have identified tumor-infiltrating stromal fibroblasts as a potent source of alternative pro-angiogenic factors. See, e.g., Dong, J. et al. VEGF-null cells require PDGFR alpha signaling-mediated stromal fibroblast recruitment for tumorigenesis. Embo J 23:2800-10 (2004); and, Orimo, A. et al. Stromal fibroblasts present in invasive human breast carcinomas promote tumor growth and angiogenesis through elevated SDF-1/CXCL12 secretion. Cell 121:335-48 (2005).

Inflammatory cells can participate in angiogenesis by secreting inflammatory cytokines, which can affect endothelial cell activation, proliferation, migration, and survival (reviewed in Albini et al. and Balkwill et al. in Albini, A., Tosetti, F., Benelli, R. & Noonan, D. M. Tumor inflammatory angiogenesis and its chemoprevention. Cancer Res 65:10637-41 (2005); and, Balkwill, F., Charles, K. A. & Mantovani, A. Smoldering and polarized inflammation in the initiation and promotion of malignant disease. Cancer Cell 7:211-7 (2005). Several tumor-infiltrating inflammatory cells secrete pro-angiogenic factors, including monocytes/macrophages (see, e.g. De Palma, M. et al. Tie2 identifies a hematopoietic lineage of proangiogenic monocytes required for tumor vessel formation and a mesenchymal population of pericyte progenitors. Cancer Cell 8:211-26 (2005); and, Yang, L. et al. Expansion of myeloid immune suppressor Gr+CD11b+ cells in tumor-bearing host directly promotes tumor angiogenesis. Cancer Cell 6:409-21 (2004)), T- and B-lymphocytes (see, e.g., Freeman, M. R. et al. Peripheral blood T lymphocytes and lymphocytes infiltrating human cancers express vascular endothelial growth factor: a potential role for T cells in angiogenesis. Cancer Res 55:4140-5 (1995)), vascular leukocytes (see, e.g., Conejo-Garcia, J. R. et al. Vascular leukocytes contribute to tumor vascularization. Blood 105:679-81 (2005)), dendritic cells (see, e.g. Conejo-Garcia, J. R. et al. Tumor-infiltrating dendritic cell precursors recruited by a beta-defensin contribute to vasculogenesis under the influence of Vegf-A. Nat Med 10:950-8 (2004)), neutrophils (see, e.g., Coussens, L. M., Tinkle, C. L., Hanahan, D. & Werb, Z. MMP-9 supplied by bone marrow-derived cells contributes to skin carcinogenesis. Cell 103:481-90 (2000)), and mast cells (see, e.g. Coussens, L. M. et al. Inflammatory mast cells up-regulate angiogenesis during squamous epithelial carcinogenesis. Genes Dev 13:382-97 (1999); and (reviewed in de Visser and Coussens in de Visser, K. E., Eichten, A. & Coussens, L. M. Paradoxical roles of the immune system during cancer development. Nat Rev Cancer 6:24-37 (2006)). It was suggested that bone marrow-derived endothelial progenitor cells (EPCs (see, e.g. Lyden, D. et al. Impaired recruitment of bone-marrow-derived endothelial and hematopoietic precursor cells blocks tumor angiogenesis and growth. Nat Med 7, 1194-201 (2001)) and perivascular progenitor cells (see, e.g., Song, S., Ewald, A. J., Stallcup, W., Werb, Z. & Bergers, G. PDGFRbeta+ perivascular progenitor cells in tumours regulate pericyte differentiation and vascular survival. Nat Cell Biol 7:870-9 (2005)) contribute to vessel formation in some experimental models of tumor growth (reviewed in Rafii et al. in Rafii, S., Lyden, D., Benezra, R., Hattori, K. & Heissig, B. Vascular and haematopoietic stem cells: novel targets for anti-angiogenesis therapy? Nat Rev Cancer 2:826-35 (2002)). Myeloid lineage hematopoietic cells, including tumor-associated macrophages (TAMs), were shown to stimulate angiogenesis either directly by secreting angiogenic factors or indirectly by producing extracellular matrix-degrading proteases, which in turn release sequestered angiogenic factors (reviewed in Lewis, C. E. & Pollard, J. W. Distinct role of macrophages in different tumor microenvironments. Cancer Research 66:605-612 (2006); and, Naldini, A. & Carraro, F. Role of inflammatory mediators in angiogenesis. Curr Drug Targets Inflamm Allergy 4:3-8 (2005)). Among the myeloid cell lineages, CD11b+Gr1+ progenitor cells isolated from the spleens of tumor-bearing mice promoted angiogenesis when co-injected with tumor cells (see, e.g. Yang, L. et al. Expansion of myeloid immune suppressor Gr+CD11b+ cells in tumor-bearing host directly promotes tumor angiogenesis. Cancer Cell 6:409-21 (2004)) and tumor-infiltrating macrophage numbers correlated with poor prognosis in some human tumors (reviewed in Balkwill et al. in Balkwill, F., Charles, K. A. & Mantovani, A. Smoldering and polarized inflammation in the initiation and promotion of malignant disease. Cancer Cell 7:211-7 (2005)). However, in another study, macrophages inhibited growth of experimental tumors in mice, suggesting their potential as anticancer therapy. See, e.g., Kohchi, C. et al. Utilization of macrophages in anticancer therapy: the macrophage network theory. Anticancer Res 24:3311-20 (2004).

Despite the relative abundance of myeloid cells and their potential to produce pro-angiogenic factors, their role in tumor resistance to anti-VEGF treatment remains unknown. There is a need to discover and understand the biological functions of myeloid cells, resistant tumors, and the factors that they produce. The present invention addresses these and other needs, as will be apparent upon review of the following disclosure.

### SUMMARY OF THE INVENTION

The invention provides methods and compositions for diagnosing and treating resistant tumors. Further, methods of treating a resistant tumor with a combination treatment are provided. For example, a method comprises administering an effective amount of a VEGF antagonist in combination with an effective amount of a second agent to a subject with the resistant tumor, wherein the second agent comprises a myeloid cell reduction agent. The myeloid cell reduction agent reduces or completes ablates myeloid cells, e.g., CD11b+Gr1+ myeloid cells. In certain embodiments of the invention, a myeloid cell reduction agent includes, but is not limited to, e.g., a Gr1 antagonist, CD11b antagonist, CD18 antagonist, an elastase inhibitor, a MCP-1 antagonist, a MIP-1 alpha antagonist, clodronate, etc. In one embodiment, the antagonist is an antibody.

The invention also provides methods for diagnosing resistant tumors and markers sets for diagnosing resistant tumors. In certain embodiments of the invention, a method includes diagnosising a resistant tumor in a subject, the method comprising providing from the subject a test cell population from a tumor of the subject or the blood of the subject; measuring the number or percentage of CD11b+Gr1+ cells in the test cell population; comparing the number or percentage of the CD11b+Gr1+ cells in the test cell population to the number or percentage of the CD11b+Gr1+ cells in a reference cell population (e.g., a cell population from an anti-VEGF sensitive tumor); and, detecting an increase in the number or percentage of CD11b+Gr1+ in the test cell population compared to reference cell population, wherein the increase in number or percentage of CD11b+Gr1+ indicates that the tumor is the resistant tumor.

In one embodiment, the method further comprises measuring spleen size of the subject and comparing the spleen size of the subject to a reference spleen size (e.g., spleen size of the subject when the subject was tumor free or when the subject was sensitive to VEGF antagonist treatment or database of spleen sizes of others who are sensitive to VEGF antagonist treatment), wherein enlarged spleen size indicates that the tumor is the resistant tumor. In yet another embodiment, the method further comprises measuring the number or percentage of a vascular surface area (VSA) of a tumor in the subject after the subject has been administered a VEGF antagonist, and comparing the number or percentage of the vascular surface area number of the tumor in the subject to a reference vascular surface area (e.g., a vascular surface area from an anti-VEGF sensitive tumor), wherein an increase in the number or percentage of the vascular surface area of the tumor indicates that the tumor is the resistant tumor. In one embodiment, the antagonist is an antibody.

In another embodiment of the invention, a method includes diagnosing a resistant tumor in a subject, the method comprising: providing from the subject a test cell population from a tumor of the subject; measuring the number or percentage of CD19 B-lymphoid cells or CD11c dendritic cells in the test cell population; comparing the number or percentage of the CD19 B-lymphoid cells or CD11c dendritic cells in the test cell population to the number or percentage of the CD19 B-lymphoid cells or CD11c dendritic cells in a reference cell population; and, detecting a decrease in the number or percentage of CD19 B-lymphoid cells or CD11c dendritic cells in the test cell population compared to reference cell population, wherein the decrease in number or percentage of CD19 B-lymphoid cells or CD11c dendritic cells indicates that the tumor is the resistant tumor.

In yet another embodiment, a method includes diagnosing a resistant tumor in a subject, the method comprising: providing from the subject a test cell population from a bone marrow of the subject; measuring the number or percentage of CD90 T-lymphoid cells, CD19 B-lymphoid cells or CD11c dendritic cells in the test cell population; comparing the number or percentage of the CD90 T-lymphoid cells, CD19 B-lymphoid cells or CD11c dendritic cells in the test cell population to the number or percentage of the CD90 T-lymphoid cells, CD19 B-lymphoid cells or CD11c dendritic cells in a reference cell population; and, detecting a decrease in the number or percentage of CD90 T-lymphoid cells, CD19 B-lymphoid cells or CD11c dendritic cells in the test cell population compared to reference cell population, wherein the decrease in number or percentage of CD90 T-lymphoid cells, CD19 B-lymphoid cells or CD11c dendritic cells indicates that the tumor is the resistant tumor.

In another embodiment of the invention, a method includes treating a resistant tumor in a subject with a combination treatment, the method comprising administering an effective amount of a VEGF antagonist in combination with an effective amount of a myeloid cell reduction agent and an effective amount of a third agent to the subject with the resistant tumor, wherein the third agent is a chemotherapeutic agent. In one embodiment, the antagonist is an antibody. In certain embodiments of the invention, a myeloid cell reduction agent includes, but is not limited to, e.g., a GR1 antagonist, CD11b antagonist, CD18 antagonist, an elastase inhibitor, a MCP-1 antagonist, a MIP-1 alpha antagonist, clodronate, etc. In yet another embodiment, the chemotherapeutic agent is 5FU, gemcitabine or a chemotherapeutic agent listed herein.

In one embodiment of the invention, a method of the invention includes providing from the subject a test cell population from a tumor of the subject; measuring expression, levels, or activity of a molecule in the test cell population; comparing the expression, levels, or activity of the molecule in the test cell population to the expression and/or activity of the molecule in a reference cell population; and, detecting an alteration in expression and/or activity of the molecule in test cell population compared to the reference cell population (e.g., a cell population from an anti-VEGF treatment sensitive tumor), wherein the molecule is nucleic acid encoding a protein or the protein encoded by the nucleic acid, thereby diagnosing or determining the resistant tumor in the subject. In certain embodiments, the protein with the altered expression and/or activity, includes, but is not limited to, e.g., IL-13R, TLR-1, Endo-Lip, FGF13, IL-4R, THBS1, Crea7, MSCA, MIP2, IL-8R, G-CSF, IL10-R2, THBSP-4 and JAM-2. The alteration in expression and/or activity can be with one or more proteins, two or more, three or more, four or more, five or more, six or more, seven or more, eight or more, nine or more, ten or more, twelve or more, thirteen or more, fourteen or more, or all of the proteins.

In certain embodiments of the invention, the expression of the molecule is upregulated and the protein includes, but is not limited to, e.g., IL-13R, TLR-1, Endo-Lip, FGF13, IL-4R, MSCA, MIP2, IL-8R and G-CSF. In certain embodiments of the invention, the expression of the molecule is downregulated and the protein includes, but is not limited to, e.g., THBS1, Crea7, IL10-R2, THBSP-4, and JAM-2.

As mentioned above, in certain embodiments of the invention, a method includes providing from the subject a test cell population from a tumor of the subject or the blood of the subject; measuring the number or percentage of CD11b+GR1+ cells in the test cell population; comparing the number or percentage of the CD11b+Gr1+ cells in the test cell population to the number or percentage of the CD11b+Gr1+ cells in a reference cell population (e.g., a cell population from an anti-VEGF sensitive tumor); and, detecting an increase in the number or percentage of CD11b+Gr1+ in the test cell population compared to reference cell population, wherein the increase in number or percentage of CD11b+Gr1+ indicates that the tumor is the resistant tumor. In one embodiment, the method further comprises detecting an alteration in expression or activity of a molecule in the test cell population compared to the reference cell population, wherein the molecule is nucleic acid encoding a protein or the protein, wherein the protein includes, but is not limited to, e.g., IL-13R, TLR-1, Endo-Lip, FGF13, IL-4R, THBS1 and Crea7. In certain embodiments, there is an alteration in expression and/or activity of one or more, two or more, three or more, four or more, five or more, six or more, seven or more, or all of the proteins.

The invention also provides for marker sets to identify resistant tumors. For example, a marker set can include two or more, three or more, four or more, five or more, six or more, seven or more, eight or more, nine or more, ten or more, twelve or more, thirteen or more, fourteen or more, or the entire set, of molecules. The molecule is a nucleic acid encoding a protein or a protein with an altered expression and/or activity and is selected from the following: IL-13R, TLR-1, Endo-Lip, FGF13, IL-4R, THBS1, Crea7, MSCA, MIP2, IL-8R, G-CSF, IL10-R2, THBSP-4 and JAM-2. In one embodiment, the molecules are derived from CD11b+Gr1+ cells and include, e.g., IL-13R, TLR-1, Endo-Lip, FGF13, IL-4R, THBS1 and Crea7. In another embodiment, the molecules are derived from resistant tumors and include, e.g., MSCA, MIP2, IL-8R, G-CSF, IL10-R2, THBSP-4 and JAM-2.

### BRIEF DESCRIPTION OF THE FIGURES

FIG. 1 Panels a-f illustrate resistance of syngeneic tumor cell lines to anti-VEGF treatment correlates with their potential to recruit BMMNCs. (a) Growth curves of xenografted LLC, EL4 and B16F1 tumors in C57BL/6, GFP bone marrow chimeric mice treated with the anti-VEGF antibody G6-23 or a control antibody (anti-Ragweed) (n=5). Treatment was started on the second day by intraperitoneal (IP) administration of control antibody, G6-23 at 10 mg/kg, twice weekly. Data shown are means±standard deviations from one representative of three independent experiments. (b) Growth of EL4 tumors in beige nude XID mice (n=10) treated with control (10 and 50 mg/kg, IP, twice weekly) or G6-23 (10 and 50 mg/kg, IP, twice weekly). Treatment was initiated on day 1 after tumor cell implantation. Statistical analysis was evaluated using the ANOVA program, *p≦0.05, **p<0.005. (c) Growth of LLC tumors (n=10) in beige nude XID mice as described for (b), G6-23 (10 and 100 mg/kg) and control (100 mg/kg) were administered IP, twice weekly, respectively. (d) FACS analysis of B16F1, EL4, and LLC tumor cell suspension treated for 14 days (n=4). Increased numbers of GFP+ BMMNCs in anti-VEGF-treated EL4 and LLC tumors were identified relative to B16F1 tumors. (e) Immunofluorescent staining of CD31+ and GFP+ cells in EL4, LLC and B16F1 tumor sections treated for 14 days with either a control or anti-VEGF antibody. A significant reduction in the amounts of CD31+ vessels and reduced presence of GFP+ cells in the stroma of B16F1 tumors was identified compared with EL4 and LLC tumors. Data shown are one representative section per group from three independent experiments. (f) Quantification of the vascular surface area (VSA) in tumor xenografts treated for 14 days. Anti-VEGF-treated B16F1 tumors displayed more pronounced reductions in vascular surface area than LLC or EL4 tumors. Data shown are means±standard error of the means of 9 to 15 sections of 3 to 5 tumors per treatment group.

FIG. 2 Panels a-d illustrate tumor admixing experiments and growth curves of B16F1 tumors admixed with with GFP+ isolated from the bone marrow and tumor of GFP-chimeric mice. (a) Growth of 2.5×10⁶ B16F1 tumor cells when admixed with 10⁶ BMMNCs isolated from EL4, LLC, or B16F1 tumor-bearing mice and treated with control antibody. As a control, BMMNCs from mice implanted with matrigel or control mice are shown. (n=5) (b) Tumor growth curves of B16F1 tumors admixed with GFP+ BMMNCs isolated from EL4, LLC or B16F1 tumor bearing mice and treated with anti-VEGF antibody. EL4 and LLC tumor-derived GFP+ bone marrow cells significantly increased growth of anti-VEGF-sensitive B16F1 tumors (n=4). Data shown in (a) and (b) are from one representative of at least two independent experiments. (c and d) Growth of 2×10⁶ B16F1 tumors when admixed with 5×10⁵ GFP positive cells isolated from 14 day old EL4, LLC or B16F1 tumors treated either with control antibody (c) or anti-VEGF, G6-23 (d).

FIG. 3 Panels a-d illustrate frequency analysis of CD11b, GR1 cell in cell migration experiments in vitro, tumor and bone marrow in vivo and their functional role in mediating resistance to anti-VEGF. CD11b+Gr1+ cells isolated from mice bearing EL4 and LLC tumors are a main BM cell population mediating resistance to anti-VEGF treatment. (a) Number of migrating CD11b+Gr1+ positive cells from freshly isolated BMMNCs following exposure to conditioned media from control or anti-VEGF-treated EL4, LLC or B16F1 tumors. Both anti-VEGF resistant tumors (EL4, LLC) induce VEGF-independent migration. (b) Multi-lineage analysis of tumor isolates from mice implanted with EL4, LLC and B16F1 tumors and treated with control or anti-VEGF. EL4 and LLC, but not B16F1 tumors, displayed a significant increase in CD11b+Gr1+ cells. Data shown are from one representative of two independent experiments. (c) Multi-lineage analysis of tumor and bone marrow isolates from mice implanted with EL4, LLC and B16F1 tumors. In contrast to tumor isolates (FIG. 3b), there was no consistent increase CD11b+ or Gr1+ cell in the bone marrow of tumor bearing mice. Data shown are from one representative of two independent experiments. (d) Growth curves of B16F1 tumors admixed with EL4- and LLC-primed, bone marrow-derived CD11b+Gr1+ cells and treated with anti-VEGF (G6-23, n=5 per group). CD11b+Gr1+ cells are necessary and sufficient to mediate resistance, as BMMNCs depleted of CD11b+Gr1+ cells displayed reduced potential to mediate resistance. Data shown are from one representative of two independent experiments. (e and f) Growth curve of B 16F1 cells admixed with tumor associated CD11b+Gr1+ cells isolated from EL4 (e) and LLC (f) tumor bearing mice. Approximately, 3×10⁵ FACS sorted CD11b+Gr1+ cells isolated from EL4 or LLC tumor bearing mice and were admixed with 3×10⁶ B16F1 cells and were implanted in C57BL/6 mice (n=5).

FIG. 4 Panels a-d illustrate gene expression analysis of bone marrow cells and tumors isolates. (a) Unsupervised cluster analysis of gene expression data from CD11b+Gr1+ cells isolated from the bone marrow of mice implanted with anti-VEGF-resistant EL4 (ER1-3), LLC (LR1-3) or anti-VEGF-sensitive B16F1 tumors (BR1-3) treated with anti-VEGF. For hierarchical clustering approach, the data was normalized to control matrigel-implanted mice. Genes down-regulated, unchanged and up-regulated are shown. A characteristic set of changes induced by anti-VEGF-resistant tumors, which is distinct from that induced by anti-VEGF-sensitive tumors, can be identified. (b) Display of genes that may be involved in the regulation of angiogenesis or myeloid cell differentiation and migration, with significant changes (p=0.05, >2 fold) in expression levels in bone marrow CD11b+GR1 + cells between anti-VEGF resistant and sensitive tumors treated with anti-VEGF for 17 days. (c) Unsupervised cluster analysis of gene expression data generated from RNA isolated from EL4, LLC and B16F1 tumors following treatment with G6-23 for 17 days. (d) Display of genes potentially involved in the regulation of angiogenesis and/or myeloid cell differentiation and migration with significant changes in expression levels (p≦0.05, fold change >2) in both anti-VEGF resistant tumors (EL4=ER1-3, LLC=LR1-3) relative to B16F1 tumors (BR1-3) following treatment with G6-23 for 17 days.

FIG. 5 Panels a-f illustrate effects of combining anti-VEGF with an antibody targeting Gr1+ myeloid cells (anti-Gr1) on growth of EL4 and LLC tumors. (a) Growth curves of EL4 tumors treated with anti-VEGF, (n=5) or anti-GR1 (n=4) either alone or in combination (anti-VEGF+anti-Gr1; combo). The number of animals in these groups is 3-4. (b) Quantification of the vascular surface area (VSA) by IHC, frequency of Gr1+ cells in the periphery and tumor and CD31+ endothelial cells (EC) by FACS and terminal tumor weights of EL4 tumors treated for 17 days as described in (a). In contrast to the almost complete reduction in circulatory GR1 cells, a 2-3 fold reduction in the tumors of anti-GR1 treated mice was found. A statistically significant difference in the terminal tumor weights between EL4 tumors with anti-VEGF alone and in combination with an anti-GR1 MAb was identified. Data are means±SEM from one representative of at least two independent experiments. (c) Growth curves of LLC tumors treated with anti-VEGF (n=5) or anti-GR1 (n=4) either alone or in combination (n=4). (d) Quantification of the vascular surface area (VSA) by IHC, frequency of Gr1+ cells in the periphery and Gr1+ and CD31+ endothelial cells (EC) in tumors by FACS and tumor weights in treated animals. There was a statistically significant difference in tumor volumes and VSA between LLC tumors treated with anti-VEGF alone and in combination with anti-GR1 (c). Data are means±SEM from one representative of at least two independent experiments. (e & f) Elastase inhibitor in combination with anti-VEGF treatment delays tumor resistance of EL4 (e) and LLC (f) tumors. Tumor volumes in the combination treatment were significantly smaller when compared to the anti-VEGF cohort. Data shown in FIG. 5 are means±standard deviations from one representative of at least two independent experiments. Statistical analysis was evaluated by ANOVA, indicates p≦0.05, ** indicates p<0.01.

FIG. 6 Panels a-b illustrate the experimental strategy use to investigate the role of BMMNCs in tumor resistant to anti-VEGF treatment and the isolation of the GFP+ cells from the tumor or bone marrow of experimental animals. Panel a schematically illustrates the experimental strategy to investigate role of BMMNCs in tumor resistant against anti-VEGF treatment. To monitor the kinetics of recruitment of BMMNCs in xenograft studies, GFP+BMMNCs were IV injected into lethally irradiated C57B1/6 mice (aI.). Next, the chimeric mice were primed by implantation of sensitive (B16F1) and resistant (EL4 and LLC) tumors in matrigel (aII.). GFP+ cells from both bone marrow (aIII.) and tumors (aIV.) of chimeric mice were isolated, admixed with B16F1 cells and injected (SC) into C57BL/6 mice. Tumor implanted animals were treated with anti-VEGF or control antibodies (aV.) in order to determine role of BMMNCs in mediating tumor resistant against anti-VEGF treatment. Panel b illustrates isolation of GFP+ cells from tumor and bone marrow of implanted mice. Using FACS sorting, GFP+ cells from both tumor and the bone marrow of implanted mice (step aII. of the strategy) were isolated (bI.). Post-sort analysis (bII.) was used to determine the purity of the GFP+ cells isolated from the tumor or bone marrow of experimental animals.

FIG. 7 illustrates CD11bGR1 purification from the bone marrow of mice implanted with EL4 and LLC tumors. BMMNCs were isolated form C57BL/6 mice implanted with EL4 or LLC cells. BMMNCs were incubated with anti-CD11b conjugated beads and passed through large-scale magnetic columns to isolate CD11b+ and CD11b− fraction. Cells from each fraction as well as an aliquot of unsorted cells were stained with CD11b and Gr1 fluorochrome conjugated antibodies to determine the purity of the cells.

FIG. 8 illustrates the elution profile of mouse lymphoma tumor lysates resistant to anti-VEGF treatment, which were treated with anti-VEGF antibody (G6-31) and loaded on a HiTrap HS column. The column was eluted in step-wise fashion with increasing salt concentration.

FIG. 9 illustrates a change in EL4 tumor size in mice after 72 hours of receiving a dose of 1) PBS liposome/ragweed, 2) PBS liposome/G6-31; 3) clodronate liposome/G6-23, 4) clodronate liposome/G6-31 or 5) clodronate liposome/PBS in the tail vein.

FIG. 10 illustrates a decrease in VEGF mRNA expression in mice that have tumors resistant to anti-VEGF treatment, when clodronate liposome was administered to mice in combination with anti-VEGF (G6-23).

FIG. 11 illustrates a decrease in KC levels in mice that have tumors resistant to anti-VEGF treatment treated with clodronate liposome and anti-VEGF (G6-23).

FIG. 12, panels A and B, illustrate that both MIP-1alpha (Panel A) and MCP-1 (Panel B) are expressed in tumor cell lines resistant to anti-VEGF treatment, where Dil(+) are endothelial cells, CD3(+) represents lymphoid cells and F4/80(+) represented macrophages.

FIG. 13, panels A and B, illustrate that MIP-1 alpha and MCP-1 have angiogenic activity in an angiogenic sprouting and capillary lumen formation assay. Panel A illustrates endothelial cell controls, where the beads were treated with VEGF and D551 for 10 days. Panel B illustrates endothelial cells treated with D551 (negative control) (top left), VEGF (negative control) (top right), 1.25 μg/ml MCP-1 and D551 (bottom left), and 1.25 μg/ml MIP-1alpha and D551 (bottom right).

FIG. 14 illustrates lineage analysis of BMNNCs from tumor bearing mice (B16F1 (a), EL4 (b) and LL2 (c)) on days 7 (p1) and days 14 (p2) of treatment with either control or anti-VEGF antibody G6-23. The insets represent cells gated for CD11b. Anti-VEGF treatment increased the levels of CD11b+ and Gr1+ cells, but none of the other cell types analyzed. Cell types that were increase between day 7 and day 14 were CXCR4+, CD11b+, CD31+ and CD11b+, CD31+ cells. In contrast, a reduction in CD19+ (B-lymphocytes) and CD90+ (T-lymphocytes) cells in LL2 and EL4, but not B16F1 tumors, between days 7 and 14 were found.

FIG. 15 illustrates multilineage analysis of GFP+ cells in the tumor and BM in mice bearing resistant and sensitive tumors. C57B1/6 mice were implanted with TIB6, B16F1, EL4 and LLC tumors and were treated with anti-VEGF or control antibodies as described. BMMNCs and tumor isolates were harvested from each mouse and were stained with antibodies against CD19 (B lymphoid), CD90 (T lymphoid), CD11c (dendritic) and also VEGF receptors (R1 and R2). Graphs represent the frequency of each subset in the tumors (a) and in the bone marrow (b) compartments.

FIG. 16. Spleen is an alternative site of homing for CD11b+Gr1+ cells in mice bearing resistant tumors. C57B1/6-GFP chimeric mice were implanted with TIB6, B16F1, EL4 and LLC tumors and were treated with anti-VEGF or control antibodies for 17 days as described. (a) Analysis of tumor bearing animals revealed a significant (p≦0.05) increase in the size of spleens in mice bearing resistant tumors. (b) Splenocytes were harvested from each mouse using mechanical disruption and were treated with lysis buffer to remove red blood cells. Spleen cells were then stained with anti-CD11b and anti-Gr1 antibodies and were analyzed in a FACS machine to investigate the frequency of CD11b+Gr1+ cells. Data analysis indicated a significant increase (p≦0.05) in the frequency of CD11b+Gr1+ cells in the spleen of mice bearing resistant tumor compared to the sensitive tumors.

* Indicates the difference in EL4 tumor bearing mice treated with anti-VEGF compared to the corresponding B16F1 and TIB6 treated animals is significant (p≦0.05). + Indicates a significant difference (p≦0.05) in LLC-tumor bearing mice treated with anti-VEGF compared to the B16F1 and TIB6 treated animals.

FIG. 17 illustrates that (a) only myeloid cells isolated from mice primed with resistant tumors are capable of mediating resistance to anti-VEGF. Graph represents growth curves of B16F1 tumors admixed with B16F1- or matrigel-primed, bone marrow-derived CD11b+Gr1+ cells and treated with anti-VEGF (n=5 per group). Tumor volume was measured for 21 days as described. (b) Induction of angiogenesis is one the mechanisms that CD11b+Gr1+ cells develop resistance to anti-VEGF treatment. VSA was analyzed in mice harboring admixture of B16F1 and CD11b+Gr1+ or CD11b-Gr1-cells. * Indicates significant difference (p≦0.05) when comparing admixture of B16F1 and CD11b+Gr1+ cells from EL4 or LLC primed mice to B16F1 admixture with CD11b-Gr1-cells isolated from the same primed animals.

FIG. 18 illustrates that distinct mechanisms govern resistance to anti-VEGF and chemotherapeutic agents. C57BL/6 mice (n=5) were implanted with EL4 (a), LLC (b), TIB6 (c) and B16F1 (d) tumors and were treated with anti-VEGF antibody, control antibody, Gemcitabine and 5FU as described. Tumor volume was measured twice a week and all mice were analyzed at day 17. * indicates a significant difference when comparing anti-VEGF treated mice to 5FU or Gemcitabine treated animals. (e) BM cells were isolated from each mouse and were stained with CD11b and GR1 fluorochrome conjugated antibodies. Graph represents the number of BM CD11b+Gr1+ cells in each treatment. (f) Tumor isolate from each mouse harvested after 17 days and was stained with the same antibodies to look at the frequency and the number of CD11b+Gr1+ cells in each tumor. Bars represent mean±SEM. * Indicates the difference in EL4 tumor bearing mice treated with anti-VEGF compared to the corresponding B16F1 and TIB6 treated animals is significant (p≦0.05). +Indicates a significant difference (p≦0.05) in LLC-tumor bearing mice treated with anti-VEGF compared to the corresponding B16F1 and TIB6 treated animals.

### DETAILED DESCRIPTION

### DEFINITIONS

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The terms “VEGF” and “VEGF-A” are used interchangeably to refer to the 165-amino acid vascular endothelial cell growth factor and related 121-, 145-, 183-, 189-, and 206-amino acid vascular endothelial cell growth factors, as described by Leung et al. Science, 246:1306 (1989), Houck et al. Mol. Endocrin., 5:1806 (1991), and, Robinson & Stringer, Journal of Cell Science, 144(5):853-865 (2001), together with the naturally occurring allelic and processed forms thereof. VEGF-A is part of a gene family including VEGF-B, VEGF-C, VEGF-D, VEGF-E, VEGF-F, and PIGF. VEGF-A primarily binds to two high affinity receptor tyrosine kinases, VEGFR-1 (Flt-1) and VEGFR-2 (Flk-1/KDR), the latter being the major transmitter of vascular endothelial cell mitogenic signals of VEGF-A. The term “VEGF” or “VEGF-A” also refers to VEGFs from non-human species such as mouse, rat, or primate. Sometimes the VEGF from a specific species is indicated by terms such as hVEGF for human VEGF or mVEGF for murine VEGF. The term “VEGF” is also used to refer to truncated forms or fragments of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

A “VEGF antagonist” refers to a molecule (peptidyl or non-peptidyl) capable of neutralizing, blocking, inhibiting, abrogating, reducing or interfering with VEGF activities including its binding to one or more VEGF receptors. VEGF antagonists include anti-VEGF antibodies and antigen-binding fragments thereof, receptor molecules and derivatives which bind specifically to VEGF thereby sequestering its binding to one or more receptors (e.g., soluble VEGF receptor proteins, or VEGF binding fragments thereof, or chimeric VEGF receptor proteins), anti-VEGF receptor antibodies and VEGF receptor antagonists such as small molecule inhibitors of the VEGFR tyrosine kinases, and fusions proteins, e.g., VEGF-Trap (Regeneron), VEGF₁₂₁-gelonin (Peregine). VEGF antagonists also include antagonist variants of VEGF, antisense molecules directed to VEGF, RNA aptamers, and ribozymes against VEGF or VEGF receptors. VEGF antagonists useful in the methods of the invention further include peptidyl or non-peptidyl compounds that specifically bind VEGF, such as anti-VEGF antibodies and antigen-binding fragments thereof, polypeptides, or fragments thereof that specifically bind to VEGF; antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. In one embodiment, the VEGF antagonist reduces or inhibits, by at least 10%, 20%, 30%, 40%, 50%, 60%, 70%, 80%, 90% or more, the expression level or biological activity of VEGF. In another embodiment, the VEGF inhibited by the VEGF antagonist is VEGF (8-109), VEGF (1-109), or VEGF₁₆₅.

The term “anti-VEGF antibody” or “an antibody that binds to VEGF” refers to an antibody that is capable of binding to VEGF with sufficient affinity and specificity that the antibody is useful as a diagnostic and/or therapeutic agent in targeting VEGF. For example, the anti-VEGF antibody of the invention can be used as a therapeutic agent in targeting and interfering with diseases or conditions wherein the VEGF activity is involved. See, e.g., U.S. Pat. Nos. 6,582,959, 6,703,020; WO98/45332; WO 96/30046; WO94/10202, WO2005/044853; EP 0666868B1; US Patent Applications 20030206899, 20030190317, 20030203409, 20050112126, 20050186208, and 20050112126; Popkov et al., Journal of Immunological Methods 288:149-164 (2004); and WO2005012359. The antibody selected will normally have a sufficiently strong binding affinity for VEGF, for example, the antibody may bind hVEGF with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. The antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B, VEGF-C, VEGF-D or VEGF-E, nor other growth factors such as PlGF, PDGF or bFGF. In one embodiment, anti-VEGF antibodies include a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599, including but not limited to the antibody known as “bevacizumab (BV),” also known as “rhuMAb VEGF” or “AVASTIN®.” Bevacizumab comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005. Additional preferred antibodies include the G6 or B20 series antibodies (e.g., G6-23, G6-31, B20-4.1), as described in PCT Application Publication No. WO2005/012359. For additional preferred antibodies see U.S. Pat. Nos. 7,060,269, 6,582,959, 6,703,020; 6,054,297; WO98/45332; WO 96/30046; WO94/10202; EP 0666868B 1; U.S. Patent Application Publication Nos. 2006009360, 20050186208, 20030206899, 20030190317, 20030203409, and 20050112126; and Popkov et al., Journal of Immunological Methods 288:149-164 (2004).

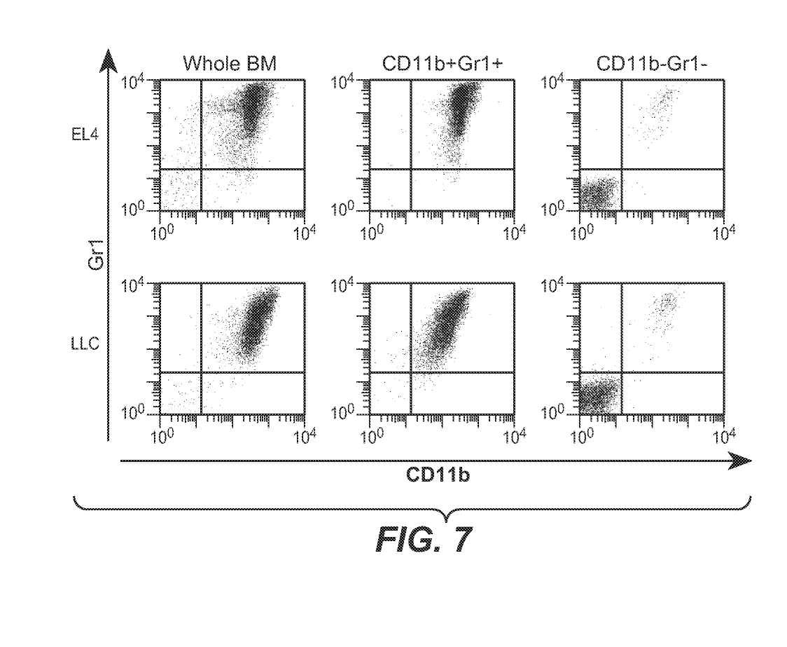

A “G6 series antibody” according to this invention, is an anti-VEGF antibody that is derived from a sequence of a G6 antibody or G6-derived antibody according to any one of FIGS. 7, 24-26, and 34-35 of PCT Application Publication No. WO2005/012359.

A “hematopoietic stem/progenitor cell” or “primitive hematopoietic cell” is one which is able to differentiate to form a more committed or mature blood cell type. “Lymphoid blood cell lineages” are those hematopoietic precursor cells which are able to differentiate to form lymphocytes (B-cells or T-cells). Likewise, “lymphopoeisis” is the formation of lymphocytes. “Erythroid blood cell lineages” are those hematopoietic precursor cells which are able to differentiate to form erythrocytes (red blood cells) and “erythropoeisis” is the formation of erythrocytes.

The phrase “myeloid blood cell lineages”, for the purposes herein, encompasses all hematopoietic progenitor cells, other than lymphoid and erythroid blood cell lineages as defined above, and “myelopoiesis” involves the formation of blood cells (other than lymphocytes and erythrocytes).

A myeloid cell population can be enriched in myeloid immune cells that are Gr1+/CD11b+ (or CD11b+Gr1+) or Gr1+/Mac-1+. These cells express a marker for myeloid cells of the macrophage lineage, CD11b, and a marker for granulocytes, Gr1. A Gr1+/CD11b+ can be selected by immunoadherent panning, for example, with an antibody to Gr1+.

A “myeloid cell reduction agent” or “myeloid cell reducing agent” refers to an agent that reduces or ablates a myeloid cell population. Typically, the myeloid cell reducing agent will reduce or ablate myeloid cells, CD11b+Gr1+, monocytes, macrophages, etc. Examples of myeloid cell reducing agents include, but are not limited to, Gr1+ antagonist, CD11b antagonist, CD18 antagonist, elastase inhibitor, MCP-1 antagonist, MIP-1 alpha antagonist, etc.

The term “GR1 antagonist” when used herein refers to a molecule which binds to GR1 and inhibits or substantially reduces a biological activity of Gr1. Non-limiting examples of GR1 antagonists include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. In one embodiment of the invention, the GR1 antagonist is an antibody, especially an anti-GR1 antibody which binds human Gr1.

The term “CD11b antagonist” when used herein refers to a molecule which binds to CD11b and inhibits or substantially reduces a biological activity of CD11b. Normally, the antagonist will block (partially or completely) the ability of a cell (e.g. immature myeloid cell) expressing the CD11b subunit at its cell surface to bind to endothelium. Non-limiting examples of CD11b antagonists include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. In one embodiment of the invention, the CD11b antagonist is an antibody, especially an anti-CD11b antibody which binds human CD11b. Exemplary CD11b antibodies include MY904 (U.S. Pat. No. 4,840,793); 1B6c (see Zhang et al., Brain Research 698:79-85 (1995)); CBRN1/5 and CBRM1/19 (WO94/08620).

The term “CD18 antagonist” when used herein refers to a molecule which binds to CD18 (preferably human CD18) and inhibits or substantially reduces a biological activity of CD18. Normally, the antagonist will block (partially or completely) the ability of a cell (e.g. a neutrophil) expressing the CD18 subunit at its cell surface to bind to endothelium. Non-limiting examples of CD18 antagonists include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. In one embodiment of the invention, the CD18 antagonist is an antibody.

Examples of anti-CD18 antibodies include MHM23 (Hildreth et al., Eur. J. Immunol. 13:202-208 (1983)); M18/2(IgG₂a; Sanches-Madrid et al., J. Exp. Med. 158:586-602 (1983)); H52 (American Type Culture Collection (ATCC) Deposit HB 10160); Mas191c and 1OT18 (Vermot Desroches et al., Scand. J. Immunol. 33:277-286 (1991)); and NA-8 (WO 94/12214). In one embodiment, the antibody is one which binds to the CD18 epitope to which either MHM23 or H52 binds. In one embodiment of the invention, the antibody has a high affinity for the CD18 polypeptide. In certain embodiments, the antibody may bind to a region in the extracellular domain of CD18 which associates with CD11b and the antibody may also dissociate a and P chains (e.g. the antibody may dissociate the CD11b and CD18 complex as is the case for the MHM23 antibody).

Monocyte chemotactic protein (MCP-1) is a chemokine involved in innate immunity and Th2 effector response, and CD4+ T cell differentiation. See, e.g., Paul, W. E., Fundamental Immunology, 5^{th} Edition, Lippincott Williams & Wilkins, (Philadelphia, 2003) at pages 801-840.

The term “MCP-1 antagonist” when used herein refers to a molecule which binds to MCP-1 and inhibits or substantially reduces a biological activity of MCP-1. Non-limiting examples of MCP-1 antagonists include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. In one embodiment of the invention, the MCP-1 antagonist is an antibody, especially an anti-MCP-1 antibody which binds human MCP-1.

Macrophage inflammatory proteins alpha and beta (MIP-1 alpha and beta are known chemokines. MIP-1 alpha is involved in innate immunity and Th1 effector response, and CD4+ T cell differentiation. See, e.g., Paul, W. E., Fundamental Immunology, 5^{th} Edition, Lippincott Williams & Wilkins, (Philadelphia, 2003) at pages 801-840.

The term “MIP-1 alpha antagonist” when used herein refers to a molecule which binds to MIP-1 alpha and inhibits or substantially reduces a biological activity of MIP-1 alpha. Non-limiting examples of MIP-1 alpha antagonists include antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. In one embodiment of the invention, the MIP-1 alpha antagonist is an antibody, especially an anti-MIP-1 alpha antibody which binds human MIP-1 alpha.

The term “antagonist” when used herein refers to a molecule capable of neutralizing, blocking, inhibiting, abrogating, reducing or interfering with the activities of a protein of the invention including its binding to one or more receptors in the case of a ligand or binding to one or more ligands in case of a receptor. Antagonists include antibodies and antigen-binding fragments thereof, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like. Antagonists also include small molecule inhibitors of a protein of the invention, and fusions proteins, receptor molecules and derivatives which bind specifically to protein thereby sequestering its binding to its target, antagonist variants of the protein, antisense molecules directed to a protein of the invention, RNA aptamers, and ribozymes against a protein of the invention.

A “blocking” antibody or an “antagonist” antibody is one which inhibits or reduces biological activity of the antigen it binds. Certain blocking antibodies or antagonist antibodies substantially or completely inhibit the biological activity of the antigen.

A “URCGPs” refers to proteins that are upregulated in CD11b+Gr+1 cells from anti-VEGF resistant tumors. URCGPs include, but are not limited to, neutropil elastase, CD14, expi, Il-13R, LDLR, TLR-1, RLF, Endo-Lip, SOCS13, FGF13, IL-4R, IL-11R, IL-1RII, IFN TM1, TNFRSF18, WNT5A, Secretory carrier membrane 1, HSP86, EGFR, EphRB2, GPCR25, HGF, Angiopoietin Like-6, Eph-RA7, Semaphorin Vlb, Neurotrophin 5, Claudin-18, MDC15, ECM and ADAMTS7B. In certain embodiment, the URCGPs refer to IL-13R, TLR-1, Endo-Lip, FGF13 and/or IL-4R.

A “DRCGPs” refers to proteins that are downregulated in CD11b+Gr1+ cells from anti-VEGF resistant tumors. DRCGPs include, but are not limited to, THBS1, Crea7, Aquaporin-1, solute carrier family protein (SCF38), apolipoprotein E (APOE), fatty acid binding protein (FABP), NCAM-140, Fibronectin type III, WIP, CD74, ICAM-2, Jagged1, 1tga4, ITGB7, TGF-BII-R, TGFb IEP, Smad4, BMPR1A, CD83, Dectin-1, CD48, E-selectin, IL-15, Suppressor of cytokine signaling 4, Cytor4 and CX3CR1. In certain embodiment, the DRCGPs refer to THBS1 and/or Crea7.

A “URRTPs” refers to proteins that are upregulated in anti-VEGF resistant tumors. URRTPs include, but are not limited to, Notch2, DMD8, MCP-1, ITGB7, G-CSF, IL-8R, MIP2, MSCA, GM-CSF, IL-1R, Meg-SF, HSP1A, IL-1R, G-CSFR, IGF2, HSP9A, FGF18, ELM1, Ledgfa, scavenger receptor type A, Macrophage C-type lectin, Pigr3, Macrophage SRT-1, G protein-coupled receptor, ScyA7, IL-1R2, IL-1 inducible protein, IL-1beta and ILIX Precuror. In certain embodiment, the URRTPs refer to MSCA, MIP2, IL-8R and/or G-CSF.

A “DRRTPs” refers to proteins that are downregulated in anti-VEGF resistant tumors. URRTPs include, but are not limited to, IL10-R2, Erb-2. 1, Caveolin3, Semcap3, INTG4, THBSP-4, ErbB3, JAM, Eng, JAM, Eng, JAM-2, Pecam1, Tlr3, TGF-B, FIZZ1, Wfs1, TP 14A, EMAP, SULF-2, Extracellular matrix 2, CTFG, TFPI, XCP2, Ramp2, ROR-alpha, Ephrin B1, SPARC-like 1, and Semaphorin A. In certain embodiments, the DRRTP refer to IL10-R2, THBSP-4, and/or JAM-2.

A “native sequence” polypeptide comprises a polypeptide having the same amino acid sequence as a polypeptide derived from nature. Thus, a native sequence polypeptide can have the amino acid sequence of naturally occurring polypeptide from any mammal. Such native sequence polypeptide can be isolated from nature or can be produced by recombinant or synthetic means. The term “native sequence” polypeptide specifically encompasses naturally occurring truncated or secreted forms of the polypeptide (e.g., an extracellular domain sequence), naturally occurring variant forms (e.g., alternatively spliced forms) and naturally occurring allelic variants of the polypeptide.

A “polypeptide chain” is a polypeptide wherein each of the domains thereof is joined to other domain(s) by peptide bond(s), as opposed to non-covalent interactions or disulfide bonds.

A polypeptide “variant” means a biologically active polypeptide having at least about 80% amino acid sequence identity with the corresponding native sequence polypeptide. Such variants include, for instance, polypeptides wherein one or more amino acid (naturally occurring amino acid and/or a non-naturally occurring amino acid) residues are added, or deleted, at the N- and/or C-terminus of the polypeptide. Ordinarily, a variant will have at least about 80% amino acid sequence identity, or at least about 90% amino acid sequence identity, or at least about 95% or more amino acid sequence identity with the native sequence polypeptide. Variants also include polypeptide fragments (e.g., subsequences, truncations, etc.), typically biologically active, of the native sequence.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif. The ALIGN-2 program should be compiled for use on a UNIX operating system, e.g., digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

The term “protein variant” as used herein refers to a variant as described above and/or a protein which includes one or more amino acid mutations in the native protein sequence. Optionally, the one or more amino acid mutations include amino acid substitution(s). Protein and variants thereof for use in the invention can be prepared by a variety of methods well known in the art. Amino acid sequence variants of a protein can be prepared by mutations in the protein DNA. Such variants include, for example, deletions from, insertions into or substitutions of residues within the amino acid sequence of protein. Any combination of deletion, insertion, and substitution may be made to arrive at the final construct having the desired activity. The mutations that will be made in the DNA encoding the variant must not place the sequence out of reading frame and preferably will not create complementary regions that could produce secondary mRNA structure. EP 75,444A.

The protein variants optionally are prepared by site-directed mutagenesis of nucleotides in the DNA encoding the native protein or phage display techniques, thereby producing DNA encoding the variant, and thereafter expressing the DNA in recombinant cell culture.

While the site for introducing an amino acid sequence variation is predetermined, the mutation per se need not be predetermined. For example, to optimize the performance of a mutation at a given site, random mutagenesis may be conducted at the target codon or region and the expressed protein variants screened for the optimal combination of desired activity. Techniques for making substitution mutations at predetermined sites in DNA having a known sequence are well-known, such as, for example, site-specific mutagenesis. Preparation of the protein variants described herein can be achieved by phage display techniques, such as those described in the PCT publication WO 00/63380.

After such a clone is selected, the mutated protein region may be removed and placed in an appropriate vector for protein production, generally an expression vector of the type that may be employed for transformation of an appropriate host.

Amino acid sequence deletions generally range from about 1 to 30 residues, optionally 1 to 10 residues, optionally 1 to 5 residues or less, and typically are contiguous.

Amino acid sequence insertions include amino- and/or carboxyl-terminal fusions of from one residue to polypeptides of essentially unrestricted length as well as intrasequence insertions of single or multiple amino acid residues. Intrasequence insertions (i.e., insertions within the native protein sequence) may range generally from about 1 to 10 residues, optionally 1 to 5, or optionally 1 to 3. An example of a terminal insertion includes a fusion of a signal sequence, whether heterologous or homologous to the host cell, to the N-terminus to facilitate the secretion from recombinant hosts.

Additional protein variants are those in which at least one amino acid residue in the native protein has been removed and a different residue inserted in its place. Such substitutions may be made in accordance with those shown in Table 1. Protein variants can also unnatural amino acids as described herein.

Amino acids may be grouped according to similarities in the properties of their side chains (in A. L. Lehninger, in Biochemisty, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

“Naturally occurring amino acid residues” (i.e. amino acid residues encoded by the genetic code) may be selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include, e.g., norleucine, ornithine, norvaline, homoserine and other amino acid residue analogues such as those described in Ellman et al. Meth. Enzym. 202:301-336 (1991) & US Patent application publications 20030108885 and 20030082575. Briefly, these procedures involve activating a suppressor tRNA with a non-naturally occurring amino acid residue followed by in vitro or in vivo transcription and translation of the RNA. See, e.g., US Patent application publications 20030108885 and 20030082575; Noren et al. Science 244:182 (1989); and, Ellman et al., supra.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In certain embodiments, the polypeptide will be purified (1) to greater than 95% by weight of polypeptide as determined by the Lowry method, or more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or silver stain. Isolated polypeptide includes the polypeptide in situ within recombinant cells since at least one component of the polypeptide's natural environment will not be present. Ordinarily, however, isolated polypeptide will be prepared by at least one purification step.

The term “antibody” is used in the broadest sense and and specifically covers monoclonal antibodies (including full length or intact monoclonal antibodies), polyclonal antibodies, multivalent antibodies, multispecific antibodies (e.g., bispecific antibodies) formed from at least two intact antibodies, and antibody fragments (see below) so long as they exhibit the desired biological activity.

Unless indicated otherwise, the expression “multivalent antibody” is used throughout this specification to denote an antibody comprising three or more antigen binding sites. The multivalent antibody is typically engineered to have the three or more antigen binding sites and is generally not a native sequence IgM or IgA antibody.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10): 1057 1062 (1995); and U.S. Pat. No. 5,641,870).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. Monoclonal antibodies are highly specific, being directed against a single antigen. In certain embodiments, a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations that typically include different antibodies directed against different determinants (epitopes), each monoclonal antibody is directed against a single determinant on the antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1991); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology) See also, for example, Li et al., Proc. Natl. Acad. Sci. USA, 103:3557-3562 (2006) regarding human antibodies generated via a human B-cell hybridoma technology.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. PNAS (USA) 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cellular toxicity.

The term “hypervariable region,” “HVR,” or “HV,” when used herein refers to the amino acid residues of an antibody which are responsible for antigen-binding. For example, the term hypervariable region refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework Region” or “FR” residues are those variable domain residues other than the hypervariable region residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

Throughout the present specification and claims, the Kabat numbering system is generally used when referring to a residue in the variable domain (approximately, residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md. (1991) expressly incorporated herein by reference). Unless stated otherwise herein, references to residues numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁ (including non-A and A allotypes), IgG₂, IgG₃, IgG₄, IgA₁, and IgA₂. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

The “light chains” of antibodies (immunoglobulins) from any vertebrate species can be assigned to one of two clearly distinct types, called kappa (κ) and lambda (λ), based on the amino acid sequences of their constant domains.

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. The Fc region of an immunoglobulin generally comprises two constant domains, a CH2 domain and a CH3 domain, and optionally comprises a CH4 domain.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

By “Fc region chain” herein is meant one of the two polypeptide chains of an Fc region.

The “CH2 domain” of a human IgG Fc region (also referred to as “Cg2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protroberance” in one chain thereof and a corresponding introduced “cavity” in the other chain thereof; see U.S. Pat. No. 5,821,333, expressly incorporated herein by reference). Such variant CH3 domains may be used to make multispecific (e.g. bispecific) antibodies as herein described.

“Hinge region” is generally defined as stretching from about Glu216, or about Cys226, to about Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In certain embodiments, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide, e.g. from about one to about ten amino acid substitutions, and preferably from about one to about five amino acid substitutions in a native sequence Fc region or in the Fc region of the parent polypeptide. The variant Fc region herein will typically possess, e.g., at least about 80% sequence identity with a native sequence Fc region and/or with an Fc region of a parent polypeptide, or at least about 90% sequence identity therewith, or at least about 95% sequence or more identity therewith.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells and neutrophils; with PBMCs and NK cells being generally preferred. The effector cells may be isolated from a native source thereof, e.g. from blood or PBMCs as described herein.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g. Ghetie and Ward., Immunol. Today 18(12):592-598 (1997); Ghetie et al., Nature Biotechnology, 15(7):637-640 (1997); Hinton et al., J. Biol Chem. 279(8):6213-6216 (2004); WO 204/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

An “affinity matured” antibody is one with one or more alterations in one or more CDRs thereof which result an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “flexible linker” herein refers to a peptide comprising two or more amino acid residues joined by peptide bond(s), and provides more rotational freedom for two polypeptides (such as two Fd regions) linked thereby. Such rotational freedom allows two or more antigen binding sites joined by the flexible linker to each access target antigen(s) more efficiently. Examples of suitable flexible linker peptide sequences include gly-ser, gly-ser-gly-ser, ala-ser, and gly-gly-gly-ser.

A “dimerization domain” is formed by the association of at least two amino acid residues (generally cysteine residues) or of at least two peptides or polypeptides (which may have the same, or different, amino acid sequences). The peptides or polypeptides may interact with each other through covalent and/or non-covalent association(s). Examples of dimerization domains herein include an Fc region; a hinge region; a CH3 domain; a CH4 domain; a CH1-CL pair; an “interface” with an engineered “knob” and/or “protruberance” as described in U.S. Pat. No. 5,821,333, expressly incorporated herein by reference; a leucine zipper (e.g. a jun/fos leucine zipper, see Kostelney et al., J. Immunol, 148: 1547-1553 (1992); or a yeast GCN4 leucine zipper); an isoleucine zipper; a receptor dimer pair (e.g., interleukin-8 receptor (IL-8R); and integrin heterodimers such as LFA-1 and GPIIIb/IIIa), or the dimerization region(s) thereof; dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF); see Arakawa et al. J. Biol. Chem. 269(45): 27833-27839 (1994) and Radziejewski et al. Biochem. 32(48): 1350 (1993)), or the dimerization region(s) thereof; a pair of cysteine residues able to form a disulfide bond; a pair of peptides or polypeptides, each comprising at least one cysteine residue (e.g. from about one, two or three to about ten cysteine residues) such that disulfide bond(s) can form between the peptides or polypeptides (hereinafter “a synthetic hinge”); and antibody variable domains. The most preferred dimerization domain herein is an Fc region or a hinge region.

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

Administration “in combination with” one or more further therapeutic agents includes simultaneous (concurrent) and/or consecutive administration in any order.

“Mammal” for purposes of treatment refers to any animal classified as a mammal, including humans, domestic and farm animals, and zoo, sports, or pet animals, such as dogs, horses, cats, cows, sheep, pigs, etc. Typically, the mammal is a human.

A “disorder” is any condition that would benefit from treatment with the molecules of the invention. This includes chronic and acute disorders or diseases including those pathological conditions which predispose the mammal to the disorder in question. Non-limiting examples of disorders to be treated herein include any form of tumor, benign and malignant tumors; vascularized tumors; hypertrophy; leukemias and lymphoid malignancies; neuronal, glial, astrocytal, hypothalamic and other glandular, macrophagal, epithelial, stromal and blastocoelic disorders; and inflammatory, angiogenic and immunologic disorders, vascular disorders that result from the inappropriate, aberrant, excessive and/or pathological vascularization and/or vascular permeability.

The term “effective amount” or “therapeutically effective amount” refers to an amount of a drug effective to treat a disease or disorder in a mammal. In the case of cancer, the effective amount of the drug may reduce the number of cancer cells; reduce the tumor size; inhibit (i.e., slow to some extent and typically stop) cancer cell infiltration into peripheral organs; inhibit (i.e., slow to some extent and typically stop) tumor metastasis; inhibit, to some extent, tumor growth; allow for treatment of the resistant tumor, and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

“Treatment” refers to both therapeutic treatment and prophylactic or preventative measures. Those in need of treatment include those already with the disorder as well as those in which the disorder is to be prevented. In certain embodiments of the invention, treatment can refer to a suppression of tumor angiogenesis and/or growth, or delayed onset of anti-VEGF resistance.

The term “biological activity” and “biologically active” with regard to a polypeptide of the invention refer to the ability of a molecule to specifically bind to and regulate cellular responses, e.g., proliferation, migration, etc. Cellular responses also include those mediated through a receptor, including, but not limited to, migration, and/or proliferation. In this context, the term “modulate” includes both promotion and inhibition.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include kidney or renal cancer, breast cancer, colon cancer, rectal cancer, colorectal cancer, lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, liver cancer, bladder cancer, cancer of the peritoneum, hepatocellular cancer, gastric or stomach cancer including gastrointestinal cancer, gastrointestinal stromal tumors (GIST), pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, urinary tract carcinomas, thyroid carcinomas, Wilm's tumor, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. “Tumor”, as used herein, refers to all neoplastic cell growth and proliferation, whether malignant or benign, and all pre-cancerous and cancerous cells and tissues.

The term “resistant tumor” refers to cancer, cancerous cells, or a tumor that does not respond completely, or loses or shows a reduced response over the course of cancer therapy to a cancer therapy comprising at least a VEGF antagonist. A resistant tumor also refers to a tumor diagnosed as resistant herein (also referred to herein as “anti-VEGF resistant tumor”). In certain embodiments, there is an increase in CD11b+Gr1+ cells in a resistant tumor compared to a tumor that is sensitive to therapy that includes at least a VEGF antagonist.

The term “anti-neoplastic composition” refers to a composition useful in treating cancer comprising at least one active therapeutic agent, e.g., “anti-cancer agent.” Examples of therapeutic agents (anti-cancer agents) include, but are limited to, e.g., chemotherapeutic agents, growth inhibitory agents, cytotoxic agents, agents used in radiation therapy, anti-angiogenesis agents, apoptotic agents, anti-tubulin agents, toxins, and other-agents to treat cancer, e.g., anti-VEGF neutralizing antibody, VEGF antagonist, anti-HER-2, anti-CD20, an epidermal growth factor receptor (EGFR) antagonist (e.g., a tyrosine kinase inhibitor), HER1/EGFR inhibitor, erlotinib, a COX-2 inhibitor (e.g., celecoxib), interferons, cytokines, antagonists (e.g., neutralizing antibodies) that bind to one or more of the ErbB2, ErbB3, ErbB4, or VEGF receptor(s), inhibitors for receptor tyrosine kinases for platet-derived growth factor (PDGF) and/or stem cell factor (SCF) (e.g., imatinib mesylate (Gleevec® Novartis)), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P and radioactive isotopes of Lu), chemotherapeutic agents, and toxins such as small molecule toxins or enzymatically active toxins of bacterial, fungal, plant or animal origin, including fragments and/or variants thereof.

A “growth inhibitory agent” when used herein refers to a compound or composition which inhibits growth of a cell in vitro and/or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factors (e.g., VEGF, VEGF-B, VEGF-C, VEGF-D, VEGF-E); placental derived growth factor (PlGF); platelet derived growth factors (PDGF, e.g., PDGFA, PDGFB, PDGFC, PDGFD); integrin; thrombopoietin (TPO); nerve growth factors such as NGF-alpha; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta and -gamma, colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20-IL-30; secretoglobin/uteroglobin; oncostatin M (OSM); a tumor necrosis factor such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

An “angiogenic factor or agent” is a growth factor which stimulates the development of blood vessels, e.g., promotes angiogenesis, endothelial cell growth, stability of blood vessels, and/or vasculogenesis, etc. For example, angiogenic factors, include, but are not limited to, e.g., VEGF and members of the VEGF family, P1GF, PDGF family, fibroblast growth factor family (FGFs), TIE ligands (Angiopoietins), ephrins, ANGPTL3, ANGPTL4, etc. It would also include factors that accelerate wound healing, such as growth hormone, insulin-like growth factor-I (IGF-I), VIGF, epidermal growth factor (EGF), CTGF and members of its family, and TGF-α and TGF-β. See, e.g. Klagsbrun and D'Amore, Annu. Rev. Physiol., 53:217-39 (1991); Streit and Detmar, Oncogene, 22:3172-3179 (2003); Ferrara & Alitalo, Nature Medicine 5(12):1359-1364 (1999); Tonini et al., Oncogene, 22:6549-6556 (2003) (e.g., Table 1 listing angiogenic factors); and, Sato Int. J. Clin. Oncol., 8:200-206 (2003).

An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent is an antibody or other antagonist to an angiogenic agent as defined above, e.g., antibodies to VEGF, antibodies to VEGF receptors, small molecules that block VEGF receptor signaling (e.g., PTK787/ZK2284, SU6668, SUTENT/SU11248 (sunitinib malate), AMG706). Anti-angiogensis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5(12):1359-1364 (1999); Tonini et al., Oncogene, 22:6549-6556 (2003) (e.g., Table 2 listing antiangiogenic factors); and, Sato Int. J. Clin. Oncol., 8:200-206 (2003) (e.g., Table 1 lists Anti-angiogenic agents used in clinical trials).

The term “immunosuppressive agent” as used herein refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); nonsteroidal antiinflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, and dexamethasone; dihydrofolate reductase inhibitors such as methotrexate (oral or subcutaneous); hydroxycloroquine; sulfasalazine; leflunomide; cytokine or cytokine receptor antibodies including anti-interferon-alpha, -beta, or -gamma antibodies, anti-tumor necrosis factor-alpha antibodies (infliximab or adalimumab), anti-TNF-alpha immunoahesin (etanercept), anti-tumor necrosis factor-beta antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 1990/08187 published Jul. 26, 1990); streptokinase; TGF-beta; streptodornase; RNA or DNA from the host; FK506; RS-61443; deoxyspergualin; rapamycin; T-cell receptor (Cohen et al., U.S. Pat. No. 5,114,721); T-cell-receptor fragments (Offner et al., Science, 251: 430-432 (1991); WO 1990/11294; Ianeway, Nature, 341: 482 (1989); and WO 1991/01133); and T-cell-receptor antibodies (EP 340,109) such as T10B9.

Examples of “nonsteroidal anti-inflammatory drugs” or “NSAIDs” are acetylsalicylic acid, ibuprofen, naproxen, indomethacin, sulindac, tolmetin, including salts and derivatives thereof, etc.

The word “label” when used herein refers to a detectable compound or composition which is conjugated directly or indirectly to the polypeptide. The label may be itself be detectable (e.g., radioisotope labels or fluorescent labels) or, in the case of an enzymatic label, may catalyze chemical alteration of a substrate compound or composition which is detectable.

An “isolated” nucleic acid molecule is a nucleic acid molecule that is identified and separated from at least one contaminant nucleic acid molecule with which it is ordinarily associated in the natural source of the polypeptide nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

Resistant Tumors

The invention is based, in part, on the discovery of cellular and molecular events leading to resistance of tumors to cancer therapy comprising at least a VEGF antagonist. A correlation between recruitment of hematopoietic bone marrow-derived cells and the development of tumor resistance to anti-VEGF treatment is shown herein.

The immune system includes hematopoietic cells, which include erythrocytes, lymphocytes, and cells of myeloid lineage. These cell types all arise from the same pluripotent stem cells. In an adult, hematopoiesis occurs in the bone marrow where stem cells divide infrequently to produce more stem cells (self-renewel) and various committed progenitor cells. It is the committed progenitor cells that will in response to specific regulator factors produce a hematopoietic cell. These regulatory factors are primarily produced by the surrounding stromal cells and in other tissues and include, for example, colony-stimulating factors (CSFs), erythropoietin (EPO), interleukin 3 (IL3), granulocyte/macrophage CSF (GM-CSF), granulocyte CSF (G-CSF), macrophage CSF (M-CSF), and STEEL factor. Alterations in the immune systems in cancer patients has been suggested to contribute to the inability or reduced ability of the immune system to mount a successful attack against the cancer, thus allowing progression of tumor growth. See, e.g., Gabrilovich et al., Antibodies to Vascular Endothelial Growth Factor Enhances the Efficacy of Cancer Immunotherapy by Improving Endogenous Dendritic Cell Function, Clinical Cancer Research 5:2963-2970 (1999).

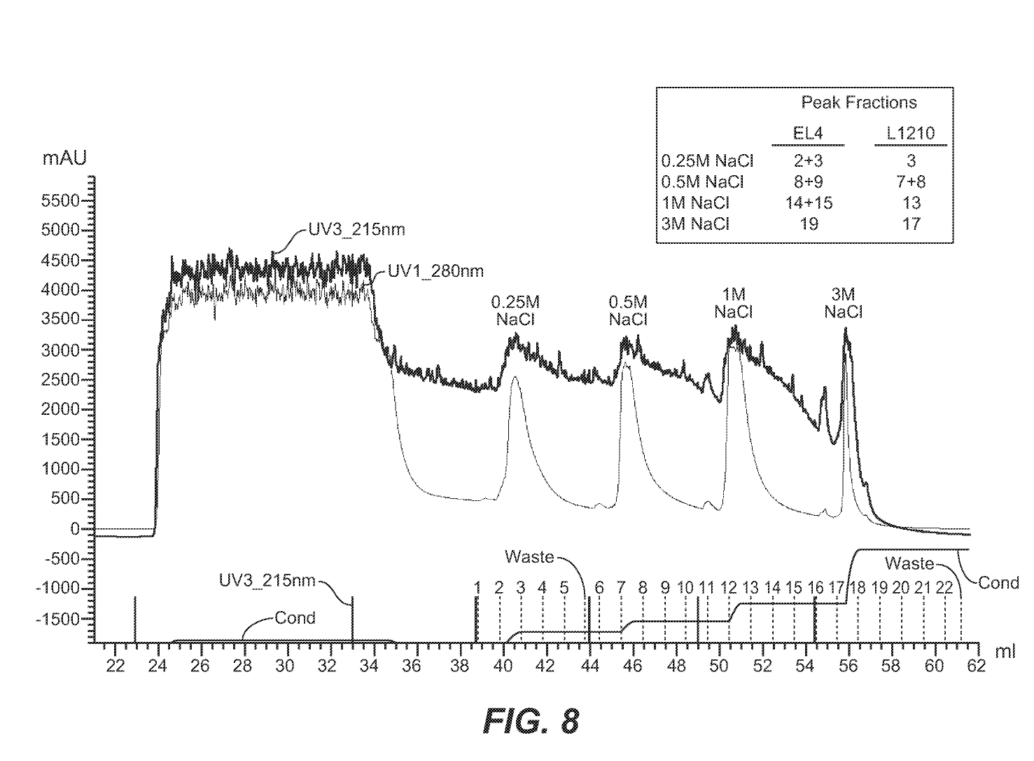

Factors produced by tumors may lead to abnormal myelopoiesis and may lead to the suppression of the immune response to the tumor. See, e.g., Kusmartsev and Gabrilovich, Immature myeloid cells and cancer-associated immune suppression. Caner Immunol Immunothera. 51:293-298 (2002). The invention provides specific factors from tumor resistant cells and CD11b+Gr1+ cells that can be involved in tumor resistance to VEGF antagonist treatment. For example, salt fractionation of resistant tumor also resulted in factors that may directly or indirectly provide resistance. See, e.g., FIG. 8 and Example 2 herein. Mobilization and activation of CD11b+Gr1+ myeloid cells can represent two steps in the development of resistance to anti-VEGF treatment.

The invention also provides combination treatment methods and compositions that use agents targeting myeloid cells and chemotherapeutic agents as described herein with anti-VEGF. These combination treatments can suppress tumor angiogenesis and growth, and/or delayed onset of anti-VEGF resistance.

CD11b+Gr1+ Cells

The CD11/CD18 family is related structurally and genetically to the larger integrin family of receptors that modulate cell adhesive interactions, which include; embryogenesis, adhesion to extracellular substrates, and cell differentiation (Hynes, R. O., Cell 48: 549-554 (1987); Kishimoto et al., Adv. Immunol. 46: 149-182 (1989); Kishimoto et al., Cell 48: 681-690 (1987); and, Ruoslahti et al., Science 238: 491-497 (1987)). Integrins are a class of membrane-spanning heterodimers comprising an α subunit in noncovalent association with a β subunit. The β subunits are generally capable of association with more than one α subunit and the heterodimers sharing a common β subunit have been classified as subfamilies within the integrin population (Larson and Springer, Structure and function of leukocyte integrins, Immunol. Rev. 114: 181-217 (1990)).

The integrin molecules of the CD11/CD18 family, and their cellular ligands, have been found to mediate a variety of cell-cell interactions, especially in inflammation. These proteins have been demonstrated to be critical for adhesive functions in the immune system (Kishimoto et al., Adv. Immunol. 46: 149-182 (1989)). Monoclonal antibodies to LFA-1 have been shown to block leukocyte adhesion to endothelial cells (Dustin et al., J. Cell. Biol. 107: 321-331 (1988); Smith et al., J. Clin. Invest. 83: 2008-2017 (1989)) and to inhibit T-cell activation (Kuypers et al., Res. Immunol., 140: 461 (1989)), conjugate formation required for antigen-specific CTL killing (Kishimoto et al., Adv. Immunol. 46: 149-182 (1989)), T. cell proliferation (Davignon et al., J. Immunol. 127: 590-595 (1981)) and NK cell killing (Krensky et al., J. Immunol. 131: 611-616 (1983)).

The CD11/CD18 family of adhesion receptor molecules comprises four highly related cell surface glycoproteins; LFA-1 (CD11a/CD18), Mac-1 (CD11b/CD18), p150.95 (CD11c/CD18) and (CD11d/CD18). Each of these heterodimers has a unique α-chain (CD11a, b, c or d) and the invariant β-chain (CD18). CD18 integrins located on leukocytes may bind to intercellular adhesion molecule-1 (ICAM-1) which is expressed on vascular endothelium and other cells, thereby mediating leukocyte adhesion and transendothelial migration. LFA-1 is present on the surface of all mature leukocytes except a subset of macrophages and is considered the major lymphoid integrin. The expression of Mac-1, p150.95 and CD11d/CD18 is predominantly confined to cells of the myeloid lineage (which include neutrophils, monocytes, macrophage and mast cells). CD11b+Gr1+ are markers also found on myeloid cells. It has been suggested that the balance between mature and immature myeloid cells is an indication for cancer and in progressive tumor growth the balance shifts toward immature myeloid cells with a decrease and function of dendritic cells. See, e.g., Kusmartsev and Gabrilovich, Immature myeloid cells and cancer-associated immune suppression. Caner Immunol Immunothera. 51:293-298 (2002). Shifting the balance, e.g., by differentiating the immature myeloid cells in tumor bearing mice improved the effect of cancer vaccines. See, Kusmartsev et al., All-trans-Retinoic Acid Eliminates Immature Myeloid Cells from Tumor-bearing Mice and Improves the Effect of Vaccination. Cancer Research 63:4441-4449 (2003). It was also observed that in cancer patients, the level of VEGF in the circulation correlated with an increase number of immature myeloid cells. See, Almand et al., Clinical significance of defective dendritic cells differentiation in cancer. Clin. Cancer Res. 6:1755 (2000).

It is shown herein that the mobilization and activation of CD11b+Gr1+ myeloid cells can result in the resistance to anti-VEGF treatment. It is also shown that bone marrow-derived CD11b+Gr1+ myeloid cells isolated from tumor-bearing mice can confer resistance in tumors to anti-VEGF treatment and conditioned media from anti-VEGF-resistant (but not anti-VEGF-sensitive tumors) stimulated migration of CD11b+Gr1+ cells.

Diagnostics

The invention also provides for methods and compositions for diagnosing a tumor resistant to VEGF antagonist treatment. In certain embodiments of the invention, methods of the invention compare the levels of expression of one or more CD11b+Gt1+ or tumor resistant nucleic acids in a test and reference cell populations. The sequence information disclosed herein, coupled with nucleic acid detection methods known in the art, allow for detection and comparison of the various disclosed transcripts. In another embodiment, methods of the invention compare the spleen size of a subject with resistant tumor compared to the reference spleen size. In one embodiment, the reference spleen size is the spleen size of the subject when the subject was tumor free or when the subject was sensitive to VEGF antagonist treatment. In another embodiment, the reference spleen size is an average spleen size of other subjects without tumor or an average spleen size of other subjects with sensitive tumors. Spleen size can be measured using methods known in the art, including, but not limited to noninvasive imaging techniques such as ultrasound, ultrasonography, one-dimensional ultrasonography (US), radionuclide scanning, computed tomography (CT) and magnetic resonance imaging. See e.g., Yang et al, West J Med.; 155(1): 47-52 (1991). In yet another embodiment, methods of invention compare the vascular surface area of a tumor in a subject with resistant tumor to a reference vascular surface area.

In certain embodiments of the invention, the invention includes providing a test cell population which includes at least one cell that is capable of expressing one or more of a molecule that is a nucleic acid encoding a protein or that is the protein, where the protein is Gr1, a neutrophil elastase, MCP-1, MIP-1 alpha, a URCGP, a DRCGP, a URRTP and/or a DRRTP. By “capable of expressing” is meant that the gene is present in an intact form in the cell and can be expressed. Expression of one, some, or all of the sequences is then detected, if present, and, measured. Using sequence information provided by the database entries for the known sequences or the chip manufacturer, sequences can be detected (if expressed) and measured using techniques well known to one of ordinary skill in the art. For example, sequences within the sequence database entries corresponding to nucleic acids that encode Gr1, a neutrophil elastase, MCP-1, MIP-1 alpha, a URCGP, DRCGP, URRTP or DRRTP, can be used to construct probes for detecting the corresponding RNA sequences in, e.g., northern blot hybridization analyses or methods which specifically, and, preferably, quantitatively amplify specific nucleic acid sequences. As another example, the sequences can be used to construct primers for specifically amplifying the nucleic acids that encode Gr1, neutrophil elastase, MCP-1, MIP-1 alpha, URCGP, DRCGP, URRTP or DRRTP sequences in, e.g. amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

Expression can be also measured at the protein level, i.e., by measuring the levels of polypeptides encoded by the gene products described herein. Such methods are well known in the art and include, e.g., immunoassays based on antibodies to proteins encoded by the genes. Expression level of one or more of the Gr1, neutrophil elastase, MCP-1, MIP-1 alpha, URCGP, DRCGP, URRTP or DRRTP sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING.RTM. methods as described in U.S. Pat. No. 5,871,697 and in Shimkets et al., Nat. Biotechnol. 17:798-803. In certain embodiments of the invention, expression of one, two or more, three or more, four or more, five or more, six or more, seven or more, eight or more, nine or more, ten or more, eleven or more, twelve or more, thirteen or more, fourteen or more, fifteen or more, 20 or more, 25 or more, or all of the sequences which encodes for Gr1, neutrophil elastase, MCP-1, MIP-1 alpha, URCGP, DRCGP, URRTP and/or DRRTP are measured.

The reference cell population includes one or more cells capable of expressing the measured Gr1, neutrophil elastase, MCP-1, MIP-1 alpha, URCGP, DRCGP, URRTP or DRRTP sequences and for which the compared parameter is known, e.g., tumor sensitive to a VEGF antagonist. In certain embodiments of the invention, Gr1, a neutrophil elastase, MCP-1, MIP-1 alpha, a URCGP, DRCGP, URRTP or DRRTP sequence in a test cell population is considered comparable in expression level to the expression level of the Gr1, neutrophil elastase, MCP-1, MIP-1 alpha,URCGP, DRCGP, URRTP or DRRTP sequence in the reference cell population if its expression level varies within a factor of less than or equal to 2.0 fold from the level of the Gr1, neutrophil elastase, MCP-1, MIP-1 alpha,URCGP, DRCGP, URRTP or DRRTP transcript in the reference cell population. In various embodiments, a Gr1, neutrophil elastase, URCGP, DRCGP, URRTP or DRRTP sequence in a test cell population can be considered altered in levels of expression if its expression level varies from the reference cell population by more than 2.0 fold from the expression level of the corresponding Gr1, neutrophil elastase, MCP-1, MIP-1 alpha, URCGP, DRCGP, URRTP or DRRTP sequence in the reference cell population.

Optionally, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations. Suitable control nucleic acids can readily be determined by one of ordinary skill in the art.

The test cell population can be any number of cells, i e., one or more cells, and can be provided in vitro, in vivo, or ex vivo.

In certain embodiments, cells in the reference cell population are derived from a tissue type as similar as possible to test cell, e.g., tumor cell. In some embodiments, the control cell is derived from the same subject as the test cell, e.g., from a region proximal to the region of origin of the test cell, or from a time point when the subject was sensitive to VEGF antagonist treatment. In one embodiment of the invention, the reference cell population is derived from a plurality of cells. For example, the reference cell population can be a database of expression patterns from previously tested cells for which tumor sensitive treatment with a VEGF antagonist is known.

Assessing Tumor Sensitivity

Recruitment of CD11b+GR1+ myeloid cells, and expression of some of the URCGP, DRCGP, URRTP or DRRTP sequences described herein is correlated with tumors resistant to VEGF antagonist treatment. Thus, in one aspect, the invention provides a method of assessing VEGF antagonist sensitivity in a subject, where VEGF antagonist sensitivity refers to the ability to treat a tumor with anti-VEGF. In one embodiment of the invention, a method includes providing one or more test cell populations from the subject that includes cells capable of expressing one or more nucleic acid sequences homologous to nucleic acid encoding a URCGP, DRCGP, URRTP or DRRTP. Expression of the sequences is compared to a reference cell population. Any reference cell population can be used, as long as the VEGF antagonist sensitivity status of the cells in the reference cell population is known. Comparison can be performed on test and reference samples measured concurrently or at temporally distinct times. An example of the latter is the use of compiled expression information, e.g., a sequence database, which assembles information about expression levels of known sequences in cells whose sensitivity status is known. In certain embodiments of the invention, the reference cell population is enriched for CD11b+Gr1+ myeloid cells. In certain embodiments of the invention, the reference cell population is enriched for tumor cells.

Diagnostic or Marker Sets

The invention also provides for marker sets to identify resistant tumors. In certain embodiments, these marker sets are provided in a kit for assessing tumor sensitivity or resistance to VEGF antagonist treatment. For example, a marker set can include two or more, three or more, four or more, five or more, six or more, seven or more, eight or more, nine or more, ten or more, twelve or more, thirteen or more, fourteen or more, fifteen or more, twenty or more, or the entire set, of molecules. The molecule is a nucleic acid encoding a protein or a protein with an altered expression and/or activity, and is selected from the following: Notch2, DMD8, MCP-1, ITGB7, G-CSF, IL-8R, MIP2, MSCA, GM-CSF, IL-1R, Meg-SF, HSP1A, IL-1R, G-CSFR, IL10-R1, Erb-2.1, Caveolin3, Semcap3, INTG4, THBSP-4, ErbB3, JAM, Eng, JAM, Eng, JAM-2, Pecam1, Tlr3, neutropil elastase, CD14, expi, II-13R, LDLR, TLR-1, RLF, Endo-Lip, SOCS13, FGF13, IL-4R, THBS1, Crea7, Aquaporin-1, SCF38, APOE, FABP, IL-11R, IL-1RII, IFN TM1, TNFRSF18, WNT5A, Secretory carrier membrane 1, HSP86, EGFR, EphRB2, GPCR25, HGF, Angiopoietin Like-6, Eph-RA7, Semaphorin V1b, Neurotrophin 5, Claudin-18, MDC15, ECM, ADAMTS7B, NCAM-140, Fibronectin type III, WIP, CD74, ICAM-2, Jagged1, 1tga4, ITGB7, TGF-BII-R, TGFb IEP, Smad4, BMPR1A, CD83, Dectin-1, CD48, E-selectin, IL-15, Suppressor of cytokine signaling 4, Cytor4, CX3CR1, IGF2, HSP9A, FGF18, ELM1, Ledgfa, scavenger receptor type A, Macrophage C-type lectin, Pigr3, Macrophage SRT-1, G protein-coupled receptor, ScyA7, IL-1R2, IL-1 inducible protein, IL-1beta, ILIX Precuror, TGF-B, FIZZ1, Wfs1, TP 14A, EMAP, SULF-2, Extracellular matrix 2, CTFG, TFPI, XCP2, Ramp2, ROR-alpha, Ephrin B1, SPARC-like 1 and Semaphorin A. In one embodiment of the invention, an antibody is provided that detects the protein. In one embodiment, the molecules are derived from CD11b+Gr1+ cells and include, e.g., IL-13R, TLR-1, Endo-Lip, FGF13, IL-4R, THBS1 and Crea7. In another embodiment, the molecules are derived from resistant tumors and include, e.g., MSCA, MIP2, IL-8R, G-CSF, IL10-R2, THBSP-4, and JAM-2.

Modulators and Uses Thereof

Modulators of VEGF, Gr1, neutrophil elastase, MCP-1, MIP-1 alpha, CD11b, CD18, URCGPs, DRCGPs, URRTPs and DRTRPs are molecules that modulate the activity of these proteins, e.g., agonists and antagonists. The term “agonist” is used to refer to peptide and non-peptide analogs of protein of the invention, and to antibodies specifically binding such proteins of the invention, provided they have the ability to provide an agonist signal. The term “agonist” is defined in the context of the biological role of the protein. In certain embodiments, agonists possess the biological activities of a native protein of the invention, e.g., for VEGF. The term “antagonist” is used to refer to molecules that have the ability to inhibit the biological activity of a protein of the invention. Antagonist can be assessed by, e.g., by inhibiting the activity of protein.

Therapeutic Uses

It is contemplated that, according to the invention, the combinations of modulators, including a VEGF antagonist, myeloid cell reduction agent, and other therapeutic agents can be used to treat various neoplasms or non-neoplastic conditions. In one embodiment, modulators, e.g., antagonists of VEGF, myeloid cell reduction agents, antagonists of URCGPs and URRTPs (“antagonists of the invention”), are used in the inhibition of cancer cell or tumor growth of resistant tumors. In certain embodiments of the invention, modulators, e.g., agonists of DRCGPs and DRRTPs (“agonists of the invention”), are used to inhibit cancer cell or tumor growth. It is contemplated that, according to the invention, antagonists of the invention can also be used to inhibit metastasis of a tumor. In certain embodiments, one or more anti-cancer agents can be administered with antagonists of the invention, and/or agonists of the invention to inhibit cancer cell or tumor growth. See also section entitled Combination Therapies herein.

Examples of neoplastic disorders to be treated with include, but are not limited to, those described herein under the terms “cancer” and “cancerous.” Non-neoplastic conditions that are amenable to treatment with antagonists of the invention include, but are not limited to, e.g., undesired or aberrant hypertrophy, arthritis, rheumatoid arthritis (RA), psoriasis, psoriatic plaques, sarcoidosis, atherosclerosis, atherosclerotic plaques, edema from myocardial infarction, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), obesity, adipose tissue mass growth, hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

Combination Therapies

As indicated above, the invention provides combined therapies in which a VEGF antagonist is administered in combination with another therapy. For example, a VEGF antagonist is administered in combination with a different agent or antagonist of the invention (and/or agonist of the invention) to treat tumor resistant to anti-VEGF treatment. In certain embodiments, additional agents, e.g., myeloid cell reduction agent, anti-cancer agents or therapeutics, anti-angiogenesis agents, or an anti-neovascularization therapeutics, can also be administered in combination with anti-VEGF and a different antagonist of the invention to treat various neoplastic or non-neoplastic conditions. In one embodiment, the neoplastic or non-neoplastic condition is characterized by pathological disorder associated with aberrant or undesired angiogenesis that is resistant to VEGF antagonist treatment. The antagonists of the invention can be administered serially or in combination with another agent that is effective for those purposes, either in the same composition or as separate compositions. Alternatively, or additionally, multiple antagonists, agents and/or agonists of the invention can be administered.

The administration of the antagonist and/or agents, e.g. myeloid cell reduction agent, of the invention can be done simultaneously, e.g., as a single composition or as two or more distinct compositions using the same or different administration routes. Alternatively, or additionally, the administration can be done sequentially, in any order. In certain embodiments, intervals ranging from minutes to days, to weeks to months, can be present between the administrations of the two or more compositions. For example, the VEGF antagonist may be administered first, followed by a different antagonist or agent, e.g., myeloid cell reduction agent, of the invention (other than a VEGF antagonist). However, simultaneous administration or administration of the different antagonist or agent of the invention first is also contemplated.

The effective amounts of therapeutic agents administered in combination with a VEGF antagonist will be at the physicians's or veterinarian's discretion. Dosage administration and adjustment is done to achieve maximal management of the conditions to be treated. The dose will additionally depend on such factors as the type of therapeutic agent to be used and the specific patient being treated. Suitable dosages for the VEGF antagonist are those presently used and can be lowered due to the combined action (synergy) of the VEGF antagonist and the different antagonist of the invention. In certain embodiments, the combination of the inhibitors potentiates the efficacy of a single inhibitor. The term “potentiate” refers to an improvement in the efficacy of a therapeutic agent at its common or approved dose. See also the section entitled Pharmaceutical Compositions herein.

Antiangiogenic therapy in relationship to cancer is a cancer treatment strategy aimed at inhibiting the development of tumor blood vessels required for providing nutrients to support tumor growth. Because angiogenesis is involved in both primary tumor growth and metastasis, the antiangiogenic treatment provided by the invention is capable of inhibiting the neoplastic growth of tumor at the primary site as well as preventing metastasis of tumors at the secondary sites, therefore allowing attack of the tumors by other therapeutics. In one embodiment of the invention, anti-cancer agent or therapeutic is an anti-angiogenic agent. In another embodiment, anti-cancer agent is a chemotherapeutic agent.

Many anti-angiogenic agents have been identified and are known in the arts, including those listed herein, e.g., listed under Definitions, and by, e.g., Carmeliet and Jain, Nature 407:249-257 (2000); Ferrara et al., Nature Reviews: Drug Discovery, 3:391-400 (2004); and Sato Int. J. Clin. Oncol., 8:200-206 (2003). See also, US Patent Application US20030055006. In one embodiment, an antagonist of the invention is used in combination with an anti-VEGF neutralizing antibody (or fragment) and/or another VEGF antagonist or a VEGF receptor antagonist including, but not limited to, for example, soluble VEGF receptor (e.g., VEGFR-1, VEGFR-2, VEGFR-3, neuropillins (e.g., NRP1, NRP2)) fragments, aptamers capable of blocking VEGF or VEGFR, neutralizing anti-VEGFR antibodies, low molecule weight inhibitors of VEGFR tyrosine kinases (RTK), antisense strategies for VEGF, ribozymes against VEGF or VEGF receptors, antagonist variants of VEGF; and any combinations thereof. Alternatively, or additionally, two or more angiogenesis inhibitors may optionally be co-administered to the patient in addition to VEGF antagonist and other agent of the invention. In certain embodiment, one or more additional therapeutic agents, e.g., anti-cancer agents, can be administered in combination with agent of the invention, the VEGF antagonist, and/or an anti-angiogenesis agent.

In certain aspects of the invention, other therapeutic agents useful for combination tumor therapy with antagonists of the invention include other cancer therapies, (e.g., surgery, radiological treatments (e.g., involving irradiation or administration of radioactive substances), chemotherapy, treatment with anti-cancer agents listed herein and known in the art, or combinations thereof). Alternatively, or additionally, two or more antibodies binding the same or two or more different antigens disclosed herein can be co-administered to the patient. Sometimes, it may be beneficial to also administer one or more cytokines to the patient.

Chemotherapeutic Agents

In certain aspects, the invention provides a method of blocking or reducing resistant tumor growth or growth of a cancer cell, by administering effective amounts of an antagonist of VEGF and an antagonist of the invention and one or more chemotherapeutic agents to a patient susceptible to, or diagnosed with, cancer. A variety of chemotherapeutic agents may be used in the combined treatment methods of the invention. An exemplary and non-limiting list of chemotherapeutic agents contemplated is provided herein under “Definition.”

As will be understood by those of ordinary skill in the art, the appropriate doses of chemotherapeutic agents will be generally around those already employed in clinical therapies wherein the chemotherapeutics are administered alone or in combination with other chemotherapeutics. Variation in dosage will likely occur depending on the condition being treated. The physician administering treatment will be able to determine the appropriate dose for the individual subject.

Relapse Tumor Growth

The invention also provides methods and compositions for inhibiting or preventing relapse tumor growth or relapse cancer cell growth. Relapse tumor growth or relapse cancer cell growth is used to describe a condition in which patients undergoing or treated with one or more currently available therapies (e.g., cancer therapies, such as chemotherapy, radiation therapy, surgery, hormonal therapy and/or biological therapy/immunotherapy, anti-VEGF antibody therapy, particularly a standard therapeutic regimen for the particular cancer) is not clinically adequate to treat the patients or the patients are no longer receiving any beneficial effect from the therapy such that these patients need additional effective therapy. As used herein, the phrase can also refer to a condition of the “non-responsive/refractory” patient, e.g., which describe patients who respond to therapy yet suffer from side effects, develop resistance, do not respond to the therapy, do not respond satisfactorily to the therapy, etc. In various embodiments, a cancer is relapse tumor growth or relapse cancer cell growth where the number of cancer cells has not been significantly reduced, or has increased, or tumor size has not been significantly reduced, or has increased, or fails any further reduction in size or in number of cancer cells. The determination of whether the cancer cells are relapse tumor growth or relapse cancer cell growth can be made either in vivo or in vitro by any method known in the art for assaying the effectiveness of treatment on cancer cells, using the art-accepted meanings of “relapse” or “refractory” or “non-responsive” in such a context. A tumor resistant to anti-VEGF treatment is an example of a relapse tumor growth.

The invention provides methods of blocking or reducing relapse tumor growth or relapse cancer cell growth in a subject by administering one or more antagonists of the invention to block or reduce the relapse tumor growth or relapse cancer cell growth in subject. In certain embodiments, the antagonist can be administered subsequent to the cancer therapeutic. In certain embodiments, the antagonists of the invention are administered simultaneously with cancer therapy, e.g., chemotherapy. Alternatively, or additionally, the antagonist therapy alternates with another cancer therapy, which can be performed in any order. The invention also encompasses methods for administering one or more inhibitory antibodies to prevent the onset or recurrence of cancer in patients predisposed to having cancer. Generally, the subject was or is concurrently undergoing cancer therapy. In one embodiment, the cancer therapy is treatment with an anti-angiogenesis agent, e.g., a VEGF antagonist. The anti-angiogenesis agent includes those known in the art and those found under the Definitions herein. In one embodiment, the anti-angiogenesis agent is an anti-VEGF neutralizing antibody or fragment (e.g., humanized A4.6.1, AVASTIN® (Genentech, South San Francisco, Calif.), Y0317, M4, G6, B20, 2C3, etc.). See, e.g., U.S. Pat. Nos. 6,582,959, 6,884,879, 6,703,020; WO98/45332; WO 96/30046; WO94/10202; EP 0666868B1; US Patent Applications 20030206899, 20030190317, 20030203409, and 20050112126; Popkov et al., Journal of Immunological Methods 288:149-164 (2004); and, WO2005012359. Additional agents can be administered in combination with VEGF antagonist and an antagonist of the invention for blocking or reducing relapse tumor growth or relapse cancer cell growth, e.g., see section entitled Combination Therapies herein.

In one embodiment, antagonists of the invention, or other therapeutics that reduce expression of Gr1, neutrophil elastase, MCP-1, MIP-1 alpha, URCGPs or URRTPs, are administered to reverse resistance or reduced sensitivity of cancer cells to certain biological (e.g., antagonist, which is an anti-VEGF antibody), hormonal, radiation and chemotherapeutic agents thereby resensitizing the cancer cells to one or more of these agents, which can then be administered (or continue to be administered) to treat or manage cancer, including to prevent metastasis.

Antibodies

Antibodies of the invention include antibodies of a protein of the invention and antibody fragment of an antibody of a protein of the invention. A polypeptide or protein of the invention includes, but not limited to, VEGF, Gr1, MCP-1, MIP-1 alpha, CD11b, CD18, a neutrophil elastase, an URCGP, a DRCGP, an URRTP, and a DRRTP. In certain aspects, a polypeptide or protein of the invention is an antibody against VEGF, Gr1, MCP-1, MIP-1 alpha, CD11b, CD18, an URCGP, a DRCGP, an URRTP, and a DRRTP, e.g., for general polypeptide or protein information provided herein.

Antibodies of the invention further include antibodies that are anti-angiogenesis agents or angiogenesis inhibitors, antibodies that are myeloid cell reduction agents, antibodies of VEGF, Gr1, neutrophil elastase, MCP-1, MIP-1 alpha, CD11b, CD18, URCGPs, DRCGPs, URRTPs, and DRRTPs, antibodies that are anti-cancer agents, or other antibodies described herein. Exemplary antibodies include, e.g. polyclonal, monoclonal, humanized, fragment, multispecific, heteroconjugated, multivalent, effecto function, etc., antibodies.

Polyclonal Antibodies

The antibodies of the invention can comprise polyclonal antibodies. Methods of preparing polyclonal antibodies are known to the skilled artisan. For example, polyclonal antibodies against an antibody of the invention are raised in animals by one or multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against a molecule of the invention, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Typically, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies

Monoclonal antibodies against an antigen described herein can be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that typically contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Typical myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against, e.g., VEGF, Gr1, neutrophil elastase, MCP-1, MIP-1 alpha, CD11b, CD18, a URCGP, a DRCGP, a URRTP or a DRRTP, or an angiogenesis molecule. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. The monoclonal antibodies may also be made by recombinant DNA methods, such as those described in U.S. Pat. No. 4,816,567. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In another embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Humanized and Human Antibodies

Antibodies of the invention can comprise humanized antibodies or human antibodies. A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immnol., 151:2623 (1993)).

It is further important that antibodies be humanized with retention of high affinity for the antigen and other favorable biological properties. To achieve this goal, according to a typical method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

Human antibodies can also be produced using various techniques known in the art, including phage display libraries (Hoogenboom and Winter, J. Mol. Biol., 227:381 (1991); Marks et al., J. Mol. Biol., 222:581 (1991)). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, K S. and Chiswell, D J., Cur Opin in Struct Biol 3:564-571 (1993). Several sources of V-gene segments can be used for phage display. For example, Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated, e.g., by essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147(1):86-95 (1991)). Human antibodies may also be generated by in vitro activated B cells (see U.S. Pat. Nos. 5,567,610 and 5,229,275).

Antibody Fragments

Antibody fragments are also included in the invention. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Patent No. 5,587,458. Fv and sFv are the only species with intact combining sites that are devoid of constant regions; thus, they are suitable for reduced nonspecific binding during in vivo use. SFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

Multispecific Antibodies (e.g., Bispecific)

Antibodies of the invention also include, e.g., multispecific antibodies, which have binding specificities for at least two different antigens. While such molecules normally will only bind two antigens (i.e. bispecific antibodies, BsAbs), antibodies with additional specificities such as trispecific antibodies are encompassed by this expression when used herein. Examples of BsAbs include those with one arm directed against a tumor cell antigen and the other arm directed against a cytotoxic trigger molecule such as anti-FcγRI/anti-CD15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3; BsAbs with one arm which binds specifically to a tumor antigen and one arm which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α(IFN-α)/anti-hybridoma idiotype, anti-CEA/anti-vinca alkaloid; BsAbs for converting enzyme activated prodrugs such as anti-CD30/anti-alkaline phosphatase (which catalyzes conversion of mitomycin phosphate prodrug to mitomycin alcohol); BsAbs which can be used as fibrinolytic agents such as anti-fibrin/anti-tissue plasminogen activator (tPA), anti-fibrin/anti-urokinase-type plasminogen activator (uPA); BsAbs for targeting immune complexes to cell surface receptors such as anti-low density lipoprotein (LDL)/anti-Fc receptor (e.g. FcγRI, FcγRII or FcγRIII); BsAbs for use in therapy of infectious diseases such as anti-CD3/anti-herpes simplex virus (HSV), anti-T-cell receptor:CD3 complex/anti-influenza, anti-FcγR/anti-HIV; BsAbs for tumor detection in vitro or in vivo such as anti-CEA/anti-EOTUBE, anti-CEA/anti-DPTA, anti-p185^{HER2}/anti-hapten; BsAbs as vaccine adjuvants; and BsAbs as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-β-galactosidase. Examples of trispecific antibodies include anti-CD3/anti-CD4/anti-CD37, anti-CD3/anti-CD5/anti-CD37 and anti-CD3/anti-CD8/anti-CD37. Bispecific antibodies can be prepared as full length antibodies or antibody fragments (e.g. F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in WO96/27011, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the VEGF receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

Heteroconjugate Antibodies

Bispecific antibodies include cross-linked or “heteroconjugate” antibodies, which are antibodies of the invention. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Multivalent Antibodies

Antibodies of the invention include a multivalent antibody. A multivalent antibody may be internalized (and/or catabolized) faster than a bivalent antibody by a cell expressing an antigen to which the antibodies bind. The antibodies of the invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ₋VD2-(X2)ₙ₋Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH—CH1-flexible linker-VH—CH1-Fc region chain; or VH—CH1-VH—CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Effector Function Engineering

It may be desirable to modify the antibody of the invention with respect to effector function, so as to enhance the effectiveness of the antibody in treating cancer, for example. For example, a cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Immunoconjugates

The invention also pertains to immunoconjugates comprising the antibody described herein conjugated to a cytotoxic agent such as a chemotherapeutic agent, toxin (e.g an enzymatically active toxin of bacterial, fungal, plant or animal origin, or fragments thereof), or a radioactive isotope (i.e., a radioconjugate). A variety of radionuclides are available for the production of radioconjugate antibodies. Examples include, but are not limited to, e.g., ²¹²Bi, ¹³¹I, ¹³¹In, ⁹⁰Y and ¹⁸⁶Re.

Chemotherapeutic agents useful in the generation of such immunoconjugates have been described above. For example, BCNU, streptozoicin, vincristine, 5-fluorouracil, the family of agents known collectively LL-E33288 complex described in U.S. Pat. Nos. 5,053,394, 5,770,710, esperamicins (U.S. Pat. No. 5,877,296), etc. (see also the definition of chemotherapeutic agents herein) can be conjugated to antibodies of the invention or fragments thereof.

For selective destruction of the tumor, the antibody may comprise a highly radioactive atom. A variety of radioactive isotopes are available for the production of radioconjugated antibodies or fragments thereof. Examples include, but are not limited to, e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, ¹¹¹In, radioactive isotopes of Lu, etc. When the conjugate is used for diagnosis, it may comprise a radioactive atom for scintigraphic studies, for example ^{99m}tc or ¹²³I, or a spin label for nuclear magnetic resonance (NMR) imaging (also known as magnetic resonance imaging, MRI), such as iodine-123, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as ^{99m}tc or ¹²³I, ¹⁸⁶Re, ¹⁸⁸Re and ¹¹¹In can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. See, e.g., Monoclonal Antibodies in Immunoscintigraphy (Chatal, CRC Press 1989) which describes other methods in detail.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, neomycin, and the tricothecenes. See, e.g., WO 93/21232 published Oct. 28, 1993.

Conjugates of the antibody and cytotoxic agent are made using a variety of bifunctional protein coupling agents such as N-succinimidyl-3-(2-pyridyldithiol)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

Alternatively, a fusion protein comprising the anti-VEGF, and/or the anti-protein of the invention antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In certain embodiments, the antibody is conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g avidin) which is conjugated to a cytotoxic agent (e.g. a radionucleotide). In certain embodiments, an immunoconjugate is formed between an antibody and a compound with nucleolytic activity (e.g., a ribonuclease or a DNA endonuclease such as a deoxyribonuclease; Dnase).

Maytansine and Maytansinoids

The invention provides an antibody of the invention, which is conjugated to one or more maytansinoid molecules. Maytansinoids are mitototic inhibitors which act by inhibiting tubulin polymerization. Maytansine was first isolated from the east African shrub Maytenus serrata (U.S. Pat. No. 3,896,11 1). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

An antibody of the invention can be conjugated to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. In one embodiment, maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Typical coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. The linkage is formed at the C-3 position of maytansinol or a maytansinol analogue.

Calicheamicin

Another immunoconjugate of interest comprises an antibody of the invention conjugated to one or more calicheamicin molecules. The calicheamicin family of antibiotics is capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ^{I}₁ (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules, or in macroemulsions. Such techniques are disclosed in Remington 's Pharmaceutical Sciences, 16th edition, Oslo, A., Ed., (1980).

Liposomes and Nanoparticles

Polypeptides of the invention can be formulated in liposomes. For example, antibodies of the invention can be formulated as immunoliposomes. Liposomes containing the antibody are prepared by methods known in the art, such as described in Epstein et al., Proc. Natl. Acad. Sci. USA, 82:3688 (1985); Hwang et al., Proc. Natl Acad. Sci. USA, 77:4030 (1980); and U.S. Pat. Nos. 4,485,045 and 4,544,545. Liposomes with enhanced circulation time are disclosed in U.S. Pat. No. 5,013,556. Generally, the formulation and use of liposomes is known to those of skill in the art.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the invention can be conjugated to the liposomes as described in Martin et al. J. Biol. Chem. 257: 286-288 (1982) via a disulfide interchange reaction. A chemotherapeutic agent (such as Doxorubicin) is optionally contained within the liposome. See Gabizon et al. J. National Cancer Inst. 81(19)1484 (1989).

Other Uses

The antibodies of the invention have various utilities. For example, antibodies of the invention may be used in diagnostic assays for, e.g., detecting the protein expression in specific cells, tissues, or serum, for cancer detection (e.g., in detecting resistant tumors), etc. In one embodiment, antibodies are used for selecting the patient population for treatment with the methods provided herein, e.g. for detecting patients with altered expression of Gr1, a neutrophil elastase, MCP-1, MIP-1 alpha, a URCGP, a DRCGP, a URRTP or a DRRTP. Various diagnostic assay techniques known in the art may be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. And Cytochem., 30:407 (1982).

Antibodies of the invention also are useful for the affinity purification of protein or fragment of a protein of the invention from recombinant cell culture or natural sources. In this process, the antibodies against the protein are immobilized on a suitable support, such a Sephadex resin or filter paper, using methods well known in the art. The immobilized antibody then is contacted with a sample containing the protein to be purified, and thereafter the support is washed with a suitable solvent that will remove substantially all the material in the sample except the protein, which is bound to the immobilized antibody. Finally, the support is washed with another suitable solvent that will release the protein from the antibody.

### Covalent Modifications to Polypeptides of the Invention

Covalent modifications of a polypeptide of the invention, e.g. a protein of the invention, an antibody of a protein of the invention, a polypeptide antagonist fragment, a fusion molecule (e.g., an immunofusion molecule), etc., are included within the scope of this invention. They may be made by chemical synthesis or by enzymatic or chemical cleavage of the polypeptide, if applicable. Other types of covalent modifications of the polypeptide are introduced into the molecule by reacting targeted amino acid residues of the polypeptide with an organic derivatizing agent that is capable of reacting with selected side chains or the N- or C-terminal residues, or by incorporating a modified amino acid or unnatural amino acid into the growing polypeptide chain, e.g., Ellman et al. Meth. Enzym. 202:301-336 (1991); Noren et al. Science 244:182 (1989); and, & US Patent application publications 20030108885 and 20030082575.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is typically performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl)carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to a polypeptide of the invention. These procedures are advantageous in that they do not require production of the polypeptide in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 published 11 Sep. 1987, and in Aplin and Wriston, CRC Crit. Rev. Biochem., pp. 259-306 (1981).

Removal of any carbohydrate moieties present on a polypeptide of the invention may be accomplished chemically or enzymatically. Chemical deglycosylation requires exposure of the polypeptide to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin, et al. Arch. Biochem. Biophys. 259:52 (1987) and by Edge et al. Anal. Biochem., 118:131 (1981). Enzymatic cleavage of carbohydrate moieties, e.g., on antibodies, can be achieved by the use of a variety of endo- and exo-glycosidases as described by Thotakura et al. Meth. Enzymol. 138:350 (1987).

Another type of covalent modification of a polypeptide of the invention comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Vectors, Host Cells and Recombinant Methods

The polypeptides of the invention can be produced recombinantly, using techniques and materials readily obtainable.

For recombinant production of a polypeptide of the invention, e.g., a protein of the invention, an antibody of a protein of the invention, e.g., anti-VEGF antibody, the nucleic acid encoding it is isolated and inserted into a replicable vector for further cloning (amplification of the DNA) or for expression. DNA encoding the polypeptide of the invention is readily isolated and sequenced using conventional procedures. For example, a DNA encoding a monoclonal antibody is isolated and sequenced, e.g., by using oligonucleotide probes that are capable of binding specifically to genes encoding the heavy and light chains of the antibody. Many vectors are available. The vector components generally include, but are not limited to, one or more of the following: a signal sequence, an origin of replication, one or more marker genes, an enhancer element, a promoter, and a transcription termination sequence.

Signal Sequence Component

Polypeptides of the invention may be produced recombinantly not only directly, but also as a fusion polypeptide with a heterologous polypeptide, which is typically a signal sequence or other polypeptide having a specific cleavage site at the N-terminus of the mature protein or polypeptide. The heterologous signal sequence selected typically is one that is recognized and processed (i.e., cleaved by a signal peptidase) by the host cell. For prokaryotic host cells that do not recognize and process the native polypeptide signal sequence, the signal sequence is substituted by a prokaryotic signal sequence selected, for example, from the group of the alkaline phosphatase, penicillinase, 1pp, or heat-stable enterotoxin II leaders. For yeast secretion the native signal sequence may be substituted by, e.g. the yeast invertase leader, α factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the polypeptide of the invention.

Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, typically primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding a polypeptide of the invention, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid Yrp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

Promotor Component

Expression and cloning vectors usually contain a promoter that is recognized by the host organism and is operably linked to a nucleic acid encoding a polypeptide of the invention. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the polypeptide of the invention.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldyhyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Transcription of polypeptides of the invention from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and typically Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the rous sarcoma virus long terminal repeat can be used as the promoter.

Enhancer Element Component

Transcription of a DNA encoding a polypeptide of this invention by higher eukaryotes is often increased by inserting an enhancer sequence into the vector. Many enhancer sequences are now known from mammalian genes (globin, elastase, albumin, α-fetoprotein, and insulin). Typically, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide-encoding sequence, but is typically located at a site 5′ from the promoter.

Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide of the invention. One useful transcription termination component is the bovine growth hormone polyadenylation region. See WO94/11026 and the expression vector disclosed therein.

Selection and Transformation of Host Cells

Suitable host cells for cloning or expressing DNA encoding the polypeptides of the invention in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Typically, the E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide of the invention-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated polypeptides of the invention are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980) ); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for polypeptide of the invention production and cultured in conventional nutrient media modified as appropriate for inducing promoters, selecting transformants, or amplifying the genes encoding the desired sequences.

Culturing the Host Cells

The host cells used to produce polypeptides of the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Polypeptide Purification

A polypeptide or protein of the invention may be recovered from a subject. When using recombinant techniques, a polypeptide of the invention can be produced intracellularly, in the periplasmic space, or directly secreted into the medium. Polypeptides of the invention may be recovered from culture medium or from host cell lysates. If membrane-bound, it can be released from the membrane using a suitable detergent solution (e.g. Triton-X 100) or by enzymatic cleavage. Cells employed in expression of a polypeptide of the invention can be disrupted by various physical or chemical means, such as freeze-thaw cycling, sonication, mechanical disruption, or cell lysing agents.

The following procedures are exemplary of suitable protein purification procedures: by fractionation on an ion-exchange column; ethanol precipitation; reverse phase HPLC; chromatography on silica, chromatography on heparin SEPHAROSE™ chromatography on an anion or cation exchange resin (such as a polyaspartic acid column, DEAE, etc.); chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of polypeptides of the invention. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular polypeptide of the invention produced.

For example, an antibody composition prepared from the cells can be purified using, for example, hydroxylapatite chromatography, gel electrophoresis, dialysis, and affinity chromatography, with affinity chromatography being the typical purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™resin (J. T. Baker, Phillipsburg, N.J.) is useful for purification. Other techniques for protein purification, e.g., those indicated above, are also available depending on the antibody to be recovered. See also, Carter et al., Bio/Technology 10: 163-167 (1992) which describes a procedure for isolating antibodies which are secreted to the periplasmic space of E. coli.

Pharmaceutical Compositions

Therapeutic formulations of agents of the invention (VEGF antagonist, myeloid cell reduction agent, URCGP antagonist, URRTP antagonist, DRCGP agonist, a DRRTP agonist, or an anti-cancer agent), and combinations thereof and described herein used in accordance with the invention are prepared for storage by mixing a molecule, e.g., polypeptide(s), having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing a polypeptide of the invention, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions. See also, e.g., U.S. Pat. No. 6,699,501, describing capsules with polyelectrolyte covering.

It is further contemplated that an agent of the invention (e.g., VEGF antagonist, myeloid cell reduction agent, chemotherapeutic agent or anti-cancer agent) can be introduced to a subject by gene therapy. Gene therapy refers to therapy performed by the administration of a nucleic acid to a subject. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 (1986)). The oligonucleotides can be modified to enhance their uptake, e.g. by substituting their negatively charged phosphodiester groups by uncharged groups. For general reviews of the methods of gene therapy, see, for example, Goldspiel et al. Clinical Pharmacy 12:488-505 (1993); Wu and Wu Biotherapy 3:87-95 (1991); Tolstoshev Ann. Rev. Pharmacol. Toxicol. 32:573-596 (1993); Mulligan Science 260:926-932 (1993); Morgan and Anderson Ann. Rev. Biochem. 62:191-217 (1993); and May TIBTECH 11:155-215 (1993). Methods commonly known in the art of recombinant DNA technology which can be used are described in Ausubel et al. eds. (1993) Current Protocols in Molecular Biology, John Wiley & Sons, NY; and Kriegler (1990) Gene Transfer and Expression, A Laboratory Manual, Stockton Press, NY.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 (1993)). For example, in vivo nucleic acid transfer techniques include transfection with viral vectors (such as adenovirus, Herpes simplex I virus, lentivirus, retrovirus, or adeno-associated virus) and lipid-based systems (useful lipids for lipid-mediated transfer of the gene are DOTMA, DOPE and DC-Chol, for example). Examples of using viral vectors in gene therapy can be found in Clowes et al. J. Clin. Invest. 93:644-651 (1994); Kiem et al. Blood 83:1467-1473 (1994); Salmons and Gunzberg Human Gene Therapy 4:129-141 (1993); Grossman and Wilson Curr. Opin. in Genetics and Devel. 3:110-114 (1993); Bout et al. Human Gene Therapy 5:3-10 (1994); Rosenfeld et al. Science 252:431-434 (1991); Rosenfeld et al. Cell 68:143-155 (1992); Mastrangeli et al. J. Clin. Invest. 91:225-234 (1993); and Walsh et al. Proc. Soc. Exp. Biol. Med. 204:289-300 (1993).

In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

Dosage and Administration

The agents of the invention (VEGF antagonist, myeloid cell reduction agent, chemotherapeutic agent, or anti-cancer agent) are administered to a human patient, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes, and/or subcutaneous administration.

In certain embodiments, the treatment of the invention involves the combined administration of a VEGF antagonist and one or more myeloid cell reduction agent or chermotherapeutic agent. In one embodiment, additional anti-cancer agents are present, e.g., one or more different anti-angiogenesis agents, one or more chemotherapeutic agents, etc. The invention also contemplates administration of multiple inhibitors, e.g., multiple antibodies to the same antigen or multiple antibodies to different proteins of the invention. In one embodiment, a cocktail of different chemotherapeutic agents is administered with the VEGF antagonist and/or one or more myeloid cell reduction agent. The combined administration includes coadministration, using separate formulations or a single pharmaceutical formulation, and/or consecutive administration in either order. For example, a VEGF antagonist may precede, follow, alternate with administration of the myeloid cell reduction agent or chemotherapeutic agent, or may be given simultaneously therewith. In one embodiment, there is a time period while both (or all) active agents simultaneously exert their biological activities.

For the prevention or treatment of disease, the appropriate dosage of the agent of the invention will depend on the type of disease to be treated, as defined above, the severity and course of the disease, whether the inhibitor is administered for preventive or therapeutic purposes, previous therapy, the patient's clinical history and response to the inhibitor, and the discretion of the attending physician. The inhibitor is suitably administered to the patient at one time or over a series of treatments. In a combination therapy regimen, the compositions of the invention are administered in a therapeutically effective amount or a therapeutically synergistic amount. As used herein, a therapeutically effective amount is such that administration of a composition of the invention and/or co-administration of VEGF antagonist and one or more other therapeutic agents, results in reduction or inhibition of the targeting disease or condition. The effect of the administration of a combination of agents can be additive. In one embodiment, the result of the administration is a synergistic effect. A therapeutically synergistic amount is that amount of VEGF antagonist and one or more other therapeutic agents, e.g., myeloid cell reduction agent, a chemotherapeutic agent or an anti-cancer agent, necessary to synergistically or significantly reduce or eliminate conditions or symptoms associated with a particular disease.

Depending on the type and severity of the disease, about 1 μg/kg to 50 mg/kg (e.g. 0.1-20 mg/kg) of VEGF antagonist or myeloid cell reduction agent, a chemotherapeutic agent, or an anti-cancer agent is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. Typically, the clinician will administered a molecule(s) of the invention until a dosage(s) is reached that provides the required biological effect. The progress of the therapy of the invention is easily monitored by conventional techniques and assays.

For example, preparation and dosing schedules for angiogenesis inhibitors, e.g., anti-VEGF antibodies, such as AVASTIN® (Genentech), may be used according to manufacturers' instructions or determined empirically by the skilled practitioner. In another example, preparation and dosing schedules for such chemotherapeutic agents may be used according to manufacturers' instructions or as determined empirically by the skilled practitioner. Preparation and dosing schedules for chemotherapy are also described in Chemotherapy Service Ed., M. C. Perry, Williams & Wilkins, Baltimore, Md. (1992).

Efficacy of the Treatment

The efficacy of the treatment of the invention can be measured by various endpoints commonly used in evaluating neoplastic or non-neoplastic disorders. For example, cancer treatments can be evaluated by, e.g., but not limited to, tumor regression, tumor weight or size shrinkage, time to progression, duration of survival, progression free survival, overall response rate, duration of response, quality of life, protein expression and/or activity. Because the anti-angiogenic agents described herein target the tumor vasculature and not necessarily the neoplastic cells themselves, they represent a unique class of anticancer drugs, and therefore can require unique measures and definitions of clinical responses to drugs. For example, tumor shrinkage of greater than 50% in a 2-dimensional analysis is the standard cut-off for declaring a response. However, the inhibitors of the invention may cause inhibition of metastatic spread without shrinkage of the primary tumor, or may simply exert a tumouristatic effect. Accordingly, approaches to determining efficacy of the therapy can be employed, including for example, measurement of plasma or urinary markers of angiogenesis and measurement of response through radiological imaging.

Articles of Manufacture

In another embodiment of the invention, an article of manufacture containing materials useful for the treatment of the disorders or diagnosing the disorders described above is provided. The article of manufacture comprises a container, a label and a package insert. Suitable containers include, for example, bottles, vials, syringes, etc. The containers may be formed from a variety of materials such as glass or plastic. In one embodiment, the container holds a composition which is effective for treating the condition and may have a sterile access port (for example the container may be an intravenous solution bag or a vial having a stopper pierceable by a hypodermic injection needle). At least one active agent in the composition is VEGF modulator and at least a second active agent is a myeloid cell reduction agent and/or a chemotherapeutic agent. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. In another embodiment, the containers hold a marker set which is diagnostic for detecting resistant tumors. At least one agent in the composition is a marker for detecting a Gr1, a neutrophil elastase, CD19, CD90,CD11c, a URCGP, a URRTP, a DRCGP and/or a DRRTP. The label on, or associated with, the container indicates that the composition is used for diagnosing a tumor resistant to VEGF antagonist treatment. The articles of manufacture of the invention may further include other materials desirable from a commercial and user standpoint, including additional active agents, other buffers, diluents, filters, needles, and syringes.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

### Tumor Resistance to Anti-VEGF Treatment Conferred by CD11b+Gr1+ Myeloid Cells

The cellular and molecular events were investigated, which lead to resistance of experimental tumors to anti-vascular endothelial growth factor (VEGF) treatment. A correlation between recruitment of bone marrow-derived cells and the development of tumor resistance to anti-VEGF treatment was found. Tumor admixing experiments demonstrated that CD11b+Gr1+ cells isolated from either bone marrow or tumors of mice bearing anti-VEGF-resistant (but not anti-VEGF-sensitive) tumors, are sufficient to confer resistance to anti-VEGF treatment. In vitro, conditioned media from anti-VEGF-resistant (but not anti-VEGF-sensitive tumors) stimulated migration of CD11b+Gr1+ cells. Recruitment of CD11b+Gr1+ cells to primary tumors represents a cellular mechanism mediating resistance to anti-VEGF treatment. Gene expression analysis of tumor-primed CD11b+Gr1+ cells identified a distinct set of genes regulated by resistant tumors. The mobilization and activation of CD11b+Gr1+ myeloid cells can represent two steps in the development of resistance to anti-VEGF treatment. Combination treatment with compounds targeting myeloid cells with anti-VEGF further suppressed tumor angiogenesis and growth and delayed onset of anti-VEGF resistance, demonstrating therapeutic benefit of combining compounds targeting myeloid cells and VEGF.

Methods

Cell Lines. The EL4, LLC, B16F1 and TIB6 (J558) tumor cell lines were obtained from American Type Culture Collection (ATCC) and maintained in tissue culture in high glucose Dulbecco's Modified Medium (DMEM) and supplemented with 10% fetal bovine serum (FBS) and 2 mM glutamine. The terms “B16F1” and “B16” are used interchangeably herein to refer to the same melanoma cell line.

Antibodies. Anti-VEGF, such as G6-23, is an antibody that binds to and neutralizes murine and human forms of VEGF. Derived from phage display technology, the IgG portion comprised murine isotype IgG2a (see, e.g. Malik, A. K. et al. Redundant roles of VEGF-B and PlGF during selective VEGF-A blockade in mice. Blood 107:550-7 (2006)) and was dosed at 10 mg/kg, IP, twice weekly unless indicated otherwise. Isotype-matched control antibody was anti-human ragweed-IgG2a (Genentech, Inc.). Anti-CD11b+ antibody (eBioSciences), anti-L-selectin (BD BioSciences) and anti-CXCR4 (Torrey Pines Lab) were used in FACS experiments. The anti-GR1 MAb (eBioSciences, CA or BD BioSciences, CA) was administered at 10 mg/kg, IP, twice weekly. Elastase Inhibitor (1 mg/mouse; eBiosciences, San Diego, Calif.) was administered IP, daily, to C57B1/6 mice (n=5) starting from one day following implantation of 5×10⁶ EL4 or LLC cells. Tumor measurement was performed twice/week and terminal tumor weights were determined as described above.

C57BL/6 GFP chimeric mouse model. C57BL/6 and enhanced green fluorescent protein (EGFP) transgenic mice (C57BL/6-TgN; ACTbEGFP;1Osb; JAX stock#003291) aged 6-8 weeks were obtained from Charles River Laboratories and Jackson Laboratories, respectively. EGFP is controlled by the β-actin promoter, abundant in all cells in EGFP transgenic mice (see, e.g., Okabe, M., Ikawa, M., Kominami, K., Nakanishi, T. & Nishimune, Y. ‘Green mice’ as a source of ubiuitous green cells. FEBS Lett 407:313-9 (1997)). C57BL/6 GFP chimeric mice were generated by lethal irradiation (11 Gy, Cs-irradiator) of C57BL/6 mice to ablate endogenous bone marrow, followed by rescue with 5×10⁶ BMMNCs isolated from EGFP transgenic mice. BMMNCs were prepared as previously described (see, Gerber, H. P. et al. VEGF regulates haematopoietic stem cell survival by an internal autocrine loop mechanism. Nature 417:954-8. (2002)). All tumor xenograft experiments in chimeric mice were performed at least 4 weeks after hematopoietic reconstitution. For tumor growth experiments, 5×10⁶ murine EL4 or LLC tumor cells were injected subcutaneously in the dorsal area. For experiments in XID mice, 1×10⁷ LLC or EL4 tumor cell were implanted.

B16F1 admixing experiments. Tumor growth studies were conducted in either beige nude XID (Harlan Sprague Dawley) or C57BL/6 mice (Jackson Lab, Bar harbor), or GFP bone marrow chimeric mice. 5×10⁶ or 10⁷ tumor cells (as indicated) were resuspended in 200 μl of MatriGel (Growth Factor reduced; BD BioSciences, CA) and injected subcutaneously in the dorsal flank region of mice. For bone marrow admixing experiments, 10⁶ BMMNCs or CD11b+Gr1+ cells isolated from bone marrow were mixed with 2.5×10⁶ B16F1 cells in 200 μl matrigel (BD BioSciences) and implanted in the flank of C57BL/6 mice immediately. For tumor GFP+/CD11b+Gr1+ admixing experiments, 2×10⁶ B16F1 cells were admixed with 3×10⁵ GFP+ cells and implanted as described. Anti-VEGF (G6-23) or control (anti-Ragweed) antibody treatment was initiated 4 days after tumor cell inoculation. Tumor size was assessed using Vernier calipers 2-3 times per week after tumors reached a palpable size. Tumor volume was determined using the Pi/6×L×W×W formula with L as the longest diameter and W the diameter at the position perpendicular to L.

Chemotherapy. C57B1/6 mice were implanted with TIB6, B16F1, EL4 and LLC cell lines. Mice did not receive any treatment for the first 4 days after implantation to allow establishment of tumor cells. Chemotherapeutic agents including 5-Flourouracil (5FU, American Pharmaceutical Partner, IL; 50 mg/kg once a week) and Gemcitabine (Eli Lilly Co, IN, 120 mg/kg twice a week) were administered IP. Tumor volume was measured twice a week and was calculated as described.

Immunohistochemistry (IHC). For immunofluorescence analysis, tumors were harvested and frozen in Optimum Cutting Temperature (OCT) medium for cryosectioning. A total of 6 μm tumor cryosections were dried at room temperature for 1 hour and fixed in acetone for 10 min at −20° C. After air-drying for 4 min at room temperature, the non-specific binding sites were blocked by incubating them for 1 hour at room temperature in 20% normal goat serum (NGS, GIBCO #16210-064; made in phosphate buffered saline (“PBS”)). Sections were stained sequentially with the following antibodies diluted in DAKO Block solution (DakoCytomation, CA), rabbit anti-GFP AlexaFluor 488 conjugate (Molecular Probes) kept at 20 μg/ml dilution for 1 hour at room temperature, goat anti-rabbit AlexaFluor 488 conjugate (Molecular Probes) kept at 1:500 dilution for 1 hour at room temperature, rat anti-mouse PECAM-1 (Clone MEC13.3; BD Pharmingen) at 1:100 dilution kept overnight at 4° C., and goat anti-rat AlexaFluor 594 conjugate (Molecular Probes) kept at 1:500 dilution for 1 hour at room temperature. The slides were washed and mounted in DAKO fluorescent mounting medium, and immunofluorescence images were collected on a Nikon microscope equipped with a Plan-Neofluar 20× objective and digitally merged.

Vascular Surface Area (VSA) measurement. Tumor vascular surface area was quantified from digital images of CD31-stained sections using a 20× objective. Typically, the pixels corresponding to stained vessels were selected by using ImageJ Software, using a predetermined threshold set at 50-70 as cut off. Contaminating (non-vessel) stray pixels were eliminated. Unless indicated otherwise, a total of 3-5 tumors per group were analyzed. A total of 15 images were taken from each of the tumor sections, each image covering an area of 1502 μm². Unless indicated otherwise, background staining of each group was determined by using a labeled control antibody and subtracted from the total vessel counts. The aggregate vessel pixel area relative to the total picture area and total area analyzed, is reported as % vessel/surface area. In one embodiment, vascular surface area can be quantified using a noninvasive quantitative method, including, but not limited to, magnetic resonance imaging, dynamic contrast-enhanced magnetic resonance imaging, computed tomography (CT) and positron emission tomography (PET). See e.g., O'Connor et al., British Journal of Cancer 96:189-195 (2007). In certain embodiments, gadolinium contrast agent and derivatives and complexes thereof can be used in the magnetic resonance imaging.

Flow cytometry. Tumors of control and anti-VEGF-treated mice were isolated and single cell suspension was generated by chopping of tumor tissues followed by treatment with a cell homogenizer (VWR). BMMNCs were flushed from femur and tibia of implated animals and underwent RBC lysis using ACK lysis buffer (Cambrex, Mass.). Peripheral blood was collected by retro-orbital bleed and 40 μl of peripheral blood was pre-treated with ACK buffer for red blood cells lysis.

Cells from BM, tumor or peripheral blood were stained with a series of monoclonal antibodies including, CD11b, Gr1, CD19, CD90, VEGFR2, CXCR4, L-Selectin2 (all from BD BioSciences, CA), VEGFR1 (R&D, CA), Tie2 (eBioSciences, CA) along with appropriate isotype control to investigate the myeloid and lymphoid fractions in each compartment. FACS data were acquired on FACS calibur and analyzed by Cell Quest Pro software (BD Biosciences).

To isolate GFP+ cells and/or CD11b+Gr1+, single cell suspension was provided from the bone marrow or tumors of implanted mice. Cells and were stained with anti-CD11b conjugated to APC and anti-GR1 conjugated to PE. Populations of GFP, GFP-, CD11b+Gr1+ and CD11b-Gr1− cells were isolated in a FACS Vantage machine and post-sort analysis ensured the purity of the population of interest in each compartment.

Microarrays. RNA from bone marrow-derived CD11b+Gr1+ cells was isolated using Qiagen Rneasy kit (Qiagen). The methods for preparation of complementary RNA (cRNA) and hybridization/scanning of the arrays were provided by Affymetrix (Affymetrix, Inc.). Five μg of total RNA was converted into double-stranded cDNA using a cDNA synthesis kit (SuperScript Choice, GIBCO/BRL) and a T7-(dT)₂₄ oligomer primer (Biosearch Technologies, Inc., Custom Synthesis). Double-stranded cDNA was purified on an affinity resin (Sample Cleanup Module Kit, Affymetrix, Inc.) and by ethanol precipitation. After second-strand synthesis, labeled cRNA was generated from the cDNA sample using a T7 RNA polymerase and biotin-labeled nucleotide in an in vitro transcription reaction (Enzo Biochem, Inc.). The labeled cRNA was purified on an affinity resin (sample cleanup module kit, Affymetrix). The amount of labeled cRNA was determined by measuring absorbance at 260 nm and using the convention that 1 OD at 260 nm corresponds to 40 μg/ml of RNA. Twenty μg of cRNA was fragmented by incubating at 94° C. for 30 min in 40 mM tris-acetate (pH 8.1), 100 mM potassium acetate, and 30 mM magnesium acetate. Samples were then hybridized to Mouse Genome 430 2.0 arrays at 45° C. for 19 hours in a rotisserie oven set at 60 rpm. Arrays were washed, stained, and scanned in the Affymetrix Fluidics station and scanner. Data analysis was performed using the Affymetrix GeneChip Analysis software or Spotfire software (Sportfire, MA). Genes with signal intensity of at least 1.5-fold higher than reference RNA were selected for further analysis. Next, genes that were significantly (p≦0.05) differentially (more than 1.5-fold in CD11b analysis and more than 2-fold in tumor analysis) expressed in EL4 and LLC samples compared with the corresponding B16F1 group were selected for final analysis. Hierarchical gene cluster analysis was performed on all tumor and CD11b data using algorithm in Spotfire (Spotfire) software.

Cell migration assay. Tumor cells were isolated as described for the FACS analysis and plated at 1×10⁶ cells/ml in DMEM, 10% FCS and 4 mM glutamine medium for 4 days in a CO₂ tissue culture incubator. Medium was concentrated proportional to the original volume using Amicon spin columns (Millipore). 600 μl of triplicate samples were used in transwell cell migration plates (Coming). 2.5×10⁴ freshly isolated BMMNCs isolated from C57BL/6 mice were resuspended in DMEM and placed on the top chamber of transwell plates, followed by incubation at 37° C. for 9 hours and the migration capacity of BMMNCs was measured by counting cells in the bottom chambers.

Statistics ANOVA was used to determine significant differences. A p-value of ≦0.05 was considered significant.

Results

Resistance to Anti-VEGF Treatment is not Caused by Suboptimal Dosing and is Lymphocyte Independent

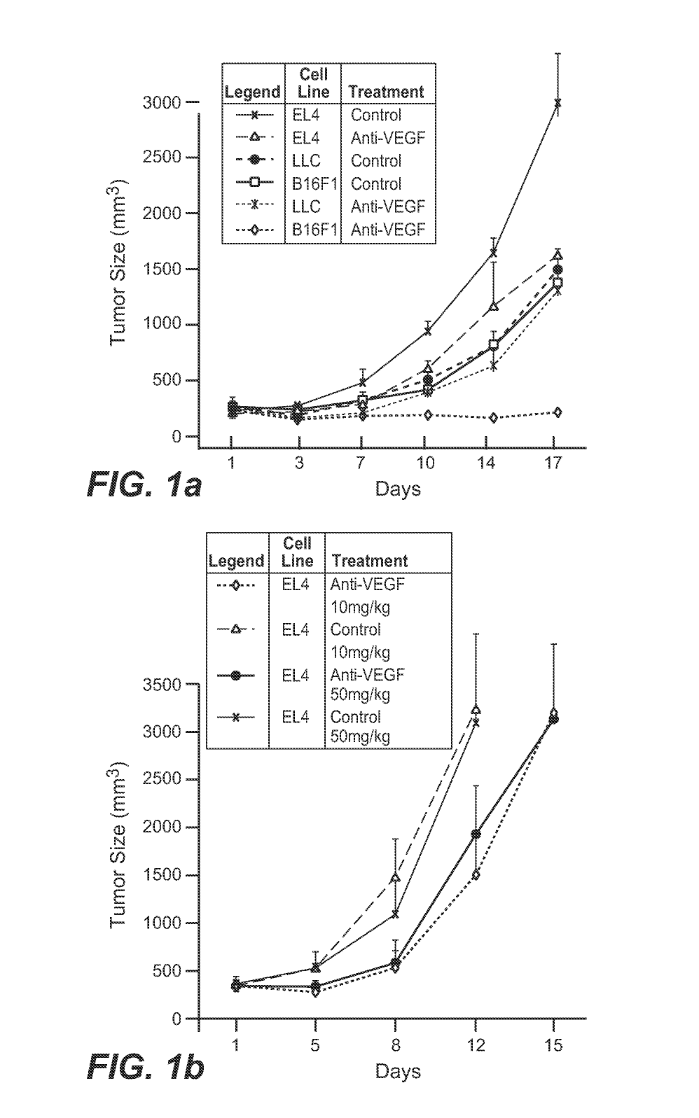

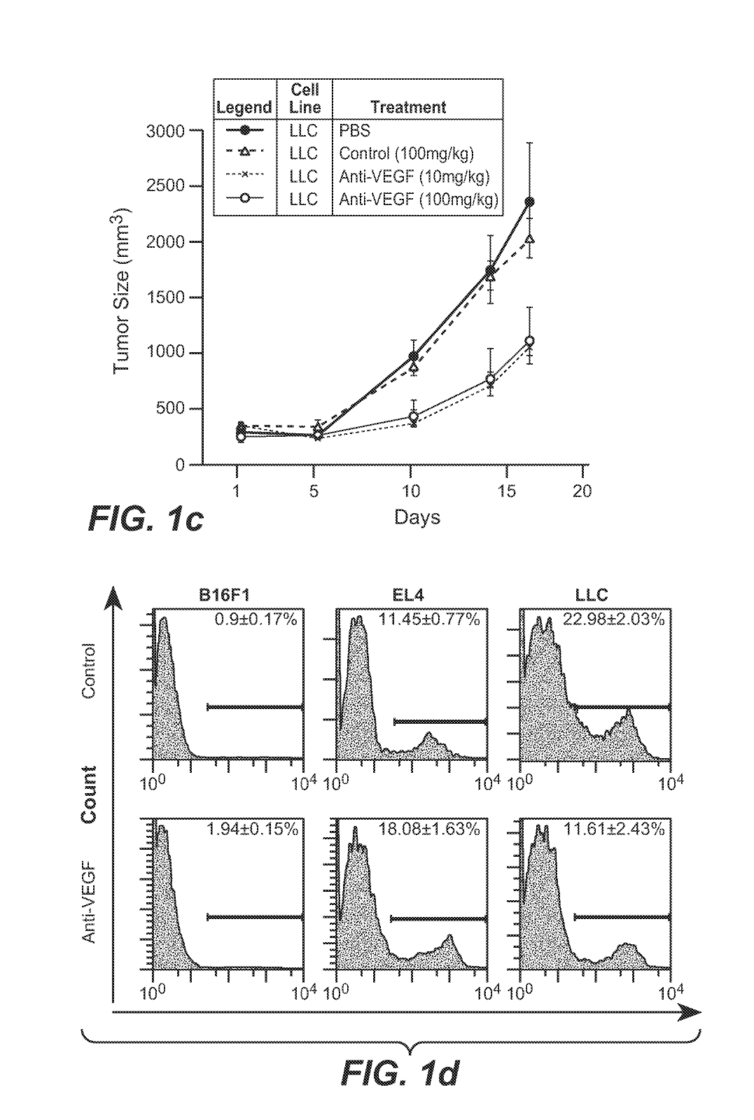

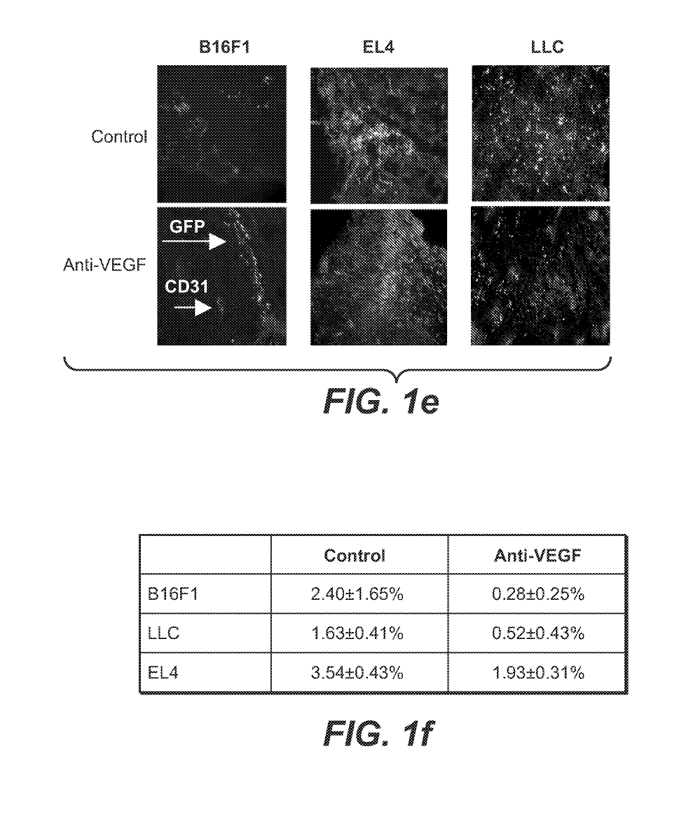

To establish an experimental model that enables assessment of the identity and relative abundance of bone marrow-derived cells (BMCs) in anti-VEGF-treated tumors, green fluorescent protein-labeled (GFP+) bone marrow mononuclear cells (BMMNCs) were adoptively transferred to lethally irradiated C57BL/6 mice (see, e.g. Okabe, M. et al., ‘Green mice’ as a source of ubiuitous green cells. FEBS Lett 407:313-9 (1997)). C57BL/6 syngeneic tumor cell lines were implanted in GFP+ bone marrow chimeric mice and the effects of a VEGF neutralizing antibody (G6-23 (see, e.g., Malik, A. K. et al. Redundant roles of VEGF-B and PlGF during selective VEGF-A blockade in mice. Blood (2005)) on tumor growth and angiogenesis were evaluated. These cell lines, included a melanoma cell line (B16F 1), two T-cell lymphoma cell lines s (EL4 and TIB6), and a Lewis lung carcinoma (LLC) cell line. The terms “B16F1” and “B16” are used interchangeably herein to refer to the same melanoma cell line. Growth of B16F1 tumors were blocked by anti-VEGF (G6-23) (FIG. 1a). In a separate experiment, growth of TIB6 tumors were also significantly blocked by anti-VEGF. However, EL4 and LLC tumors were only transiently suppressed and after an initial growth delay, tumors started expanding rapidly (FIG. 1a). Similarly, G6-23 treatment of EL4 (FIG. 1b) and LLC tumors (FIG. 1c) implanted in immunocompromised beige nude X-linked immunodeficiency (XID) mice, resulted in only transient tumor growth delays at all doses tested. These findings indicate that resistance to anti-VEGF treatment occurs in a T- and B-lymphocyte-independent manner. Resistance of EL4 and LLC tumors was not caused by suboptimal doses of anti-VEGF antibody in this model (FIG. 1b and 1c).

Lack of Bone Marrow Derived Endothelial Cell Progenitors (BM-EPCs) in the Vasculature of Anti-VEGF-Sensitive and -Resistant Tumors.

Fluorescence-activated cell sorter (FACS) analysis of EL4 and LLC tumor isolates revealed increased (p≦0.05) frequency of GFP+ bone marrow cells in resistant tumors in both anti-VEGF and control treated mice compared to anti-VEGF sensitive tumors suggesting that resistance to anti-VEGF treatment is associated with the recruitment of BMMNCs (FIG. 1d). To elucidate whether infiltrating BMMNCs directly contribute in tumor vasculature, platelet endothelial-cell adhesion molecule (CD31, PECAM)/GFP double staining was used to quantify microvessel surface areas and the numbers of GFP+/CD31+ (PECAM) EPCs in tumor sections. On day 14 of treatment, and irrespective of tumor type, the vast majority of CD31+ vascular structures in anti-VEGF- or control-treated tumors were devoid of GFP+ expression (FIG. 1e). These findings suggest that BM-EPC recruitment to tumor vasculature does not contribute directly to the formation of tumor vascularture in anti-VEGF resistant or sensitive tumors. Anti-VEGF-treated EL4 and LLC tumors displayed a 2-3-fold reduction in vascular surface area compared with control-treated tumors (FIG. 1f), correlating with a similar reduction in tumor weights. The reduction in CD31+ vessels following anti-VEGF treatment was greater in anti-VEGF-sensitive B16F1 tumors than anti-VEGF-resistant EL4 and LLC tumors. In addition, analysis of vascular surface area (VSA) displayed a significant (p≦0.05) reduction in CD31+ vessels following anti-VEGF treatment in sensitive tumors compared to resistant ones (FIG. 1f).

Recruitment and Priming of BMMNCs are Important for Anti-VEGF Resistance

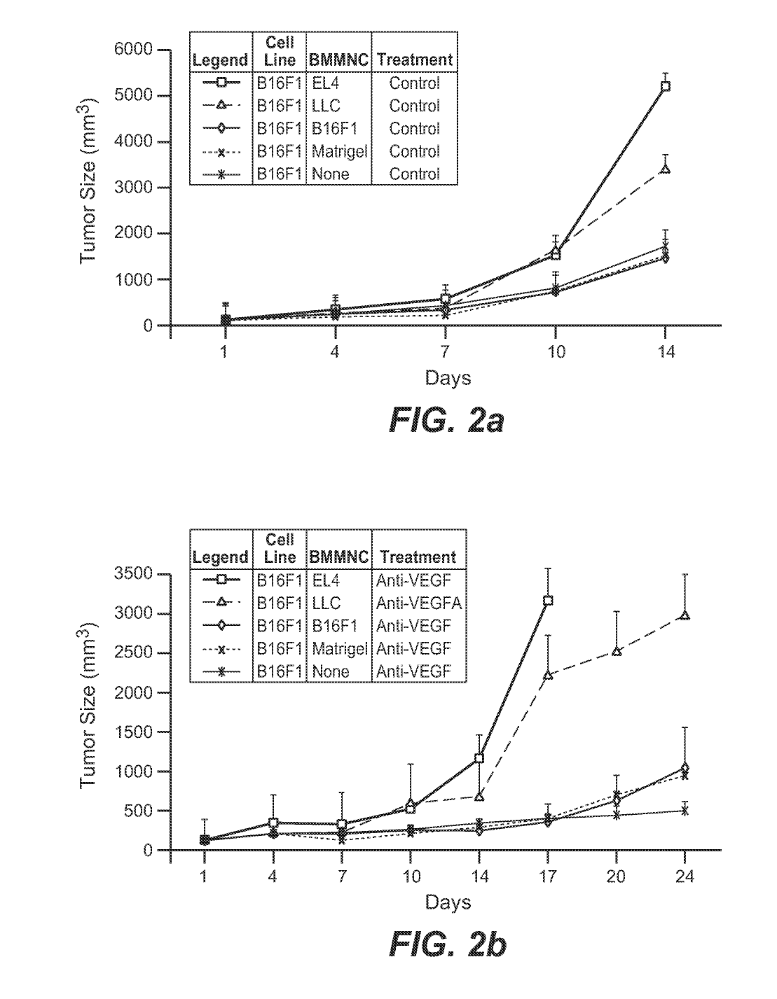

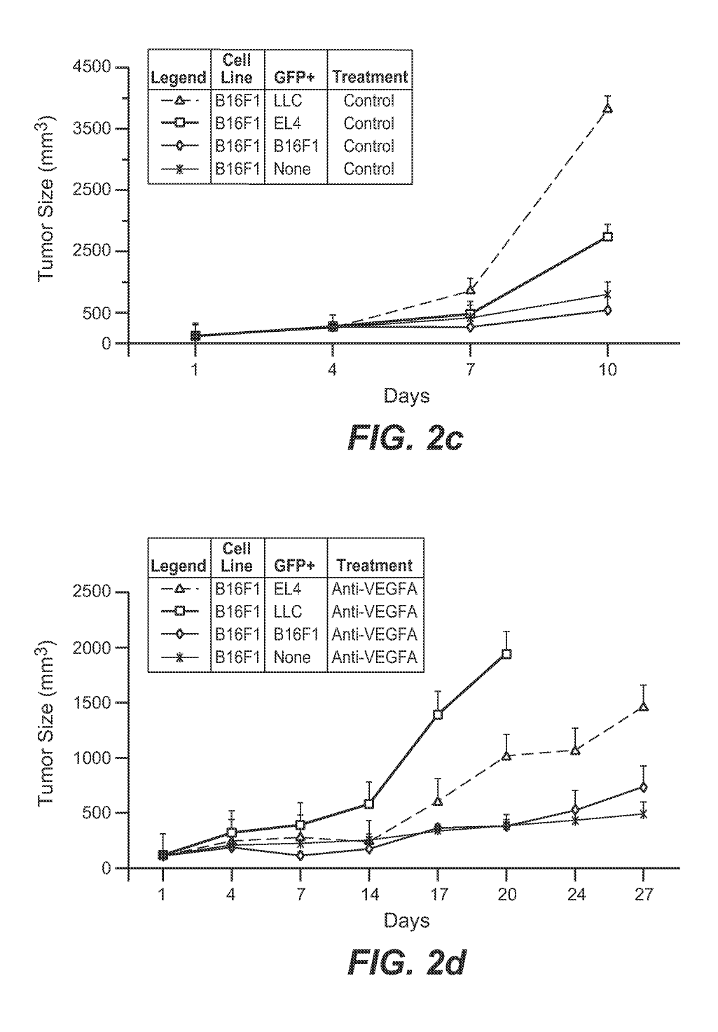

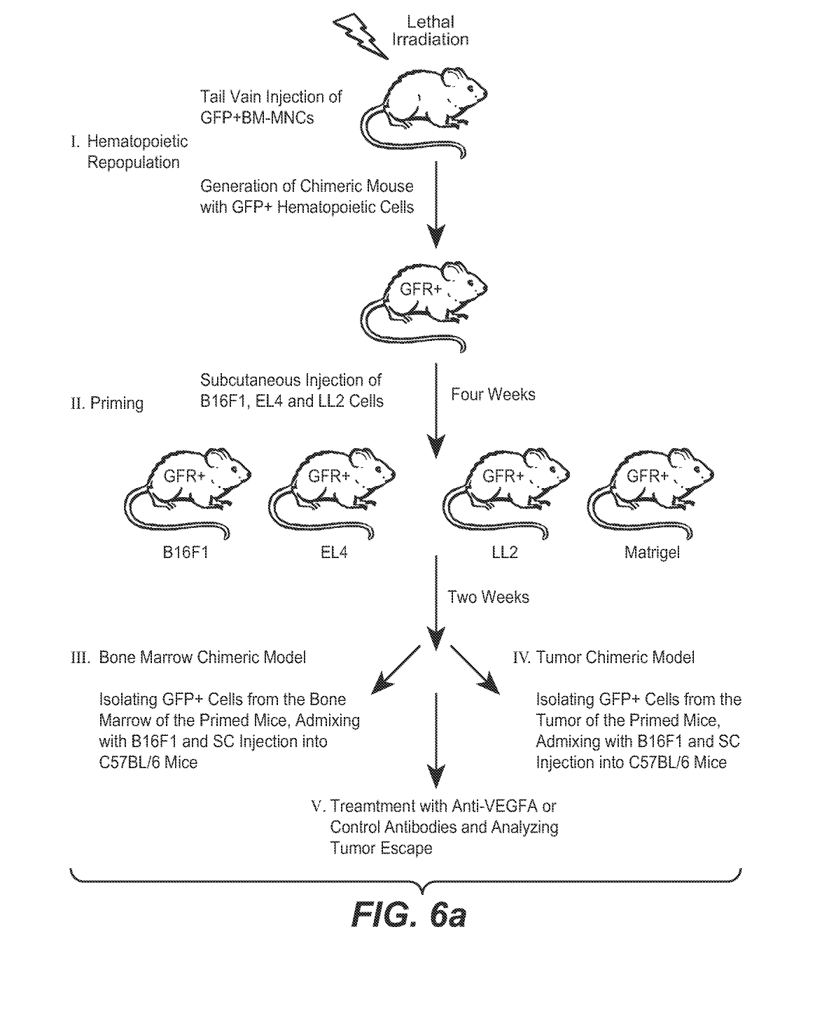

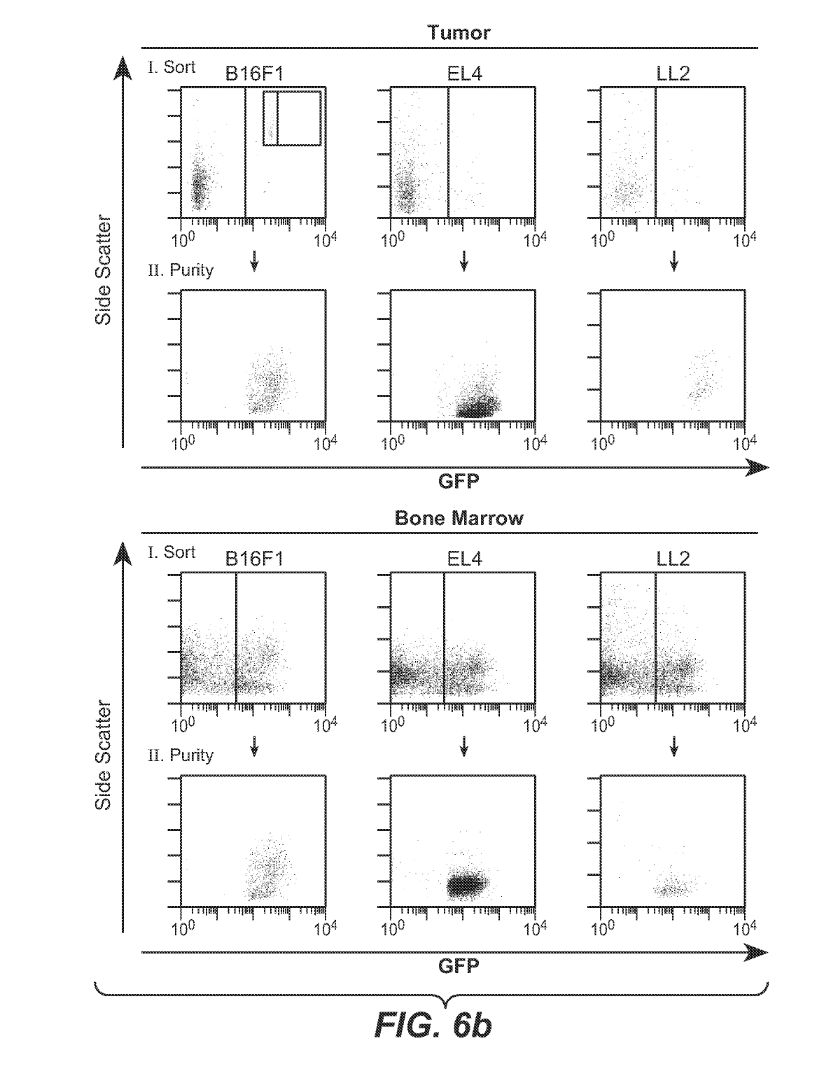

Tumor admixing experiments were conducted with anti-VEGF-sensitive B16F1 tumors to assess the functional relevance of GFP+ BMMNCs in the development of resistance to anti-VEGF treatment. See FIG. 6a and b and FIG. 7 for the experimental design and cellular purity. To perform bone marrow and tumor chimeric experiments, GFP+ cells were isolated from the tumors or the bone marrow of mice implanted with resistant and sensitive tumors. Post-sort analysis ensured the purity of GFP+ cells in each compartment. Admixing B16F1 with BMMNCs primed by resistant tumors revealed a significant (p≦0.05) growth stimulatory effect (FIG. 2a, b). In contrast, growth rates of B16F1 tumors, when admixed with BMMNCs primed by B16F1 tumors or control matrigel implants, were not significantly altered (FIG. 2a, b). BMMNCs isolated from the tibia of mice carrying EL4 and LLC tumors, admixed with B16F1 tumors, increased tumor growth rates significantly compared with BMMNCs from matrigel- or control-implanted mice (FIG. 2a). The differences in tumor growth rates were more pronounced in anti-VEGF-treated groups (FIG. 2b) than control antibody-treated groups (FIG. 2a). In contrast, growth rates in B16F1 tumors were not significantly increased when admixed with BMMNCs primed with B16F1 tumors or control matrigel, irrespective of treatment (FIG. 2a, b). Similarly, GFP+ cells isolated from EL4 and LLC tumors after 14 days of growth were sufficient to mediate resistance to anti-VEGF treatment when admixed with the anti-VEGF sensitive B16F1 tumors (FIG. 2c, d). GFP+ BMMNCs or CD11b+Gr1+ cells did not give rise to tumors when implanted alone, demonstrating the absence of contaminating tumor cells. Physical proximity of BMMNCs and anti-VEGF-sensitive tumors alone is insufficient to induce resistance and priming of bone marrow cells by anti-VEGF-resistant tumors is needed in mediating tumor resistance. Combined, these data indicate that both recruitment of BMMNCs to tumors and priming by resistant tumors are two of the steps in the cascade of events leading to the development of resistance to anti-VEGF treatment.

CD11b+Gr1+ Cells Primed by Resistant Tumors are the Major Bone Marrow Population that Mediate Anti-VEGF Resistance

BMMNCs comprise a heterogeneous population including cells of primitive, myeloid and lymphoid lineages. Morrison, S. J. et al, Annu Rev Cell Dev Biol, 11:35-71 (1995).

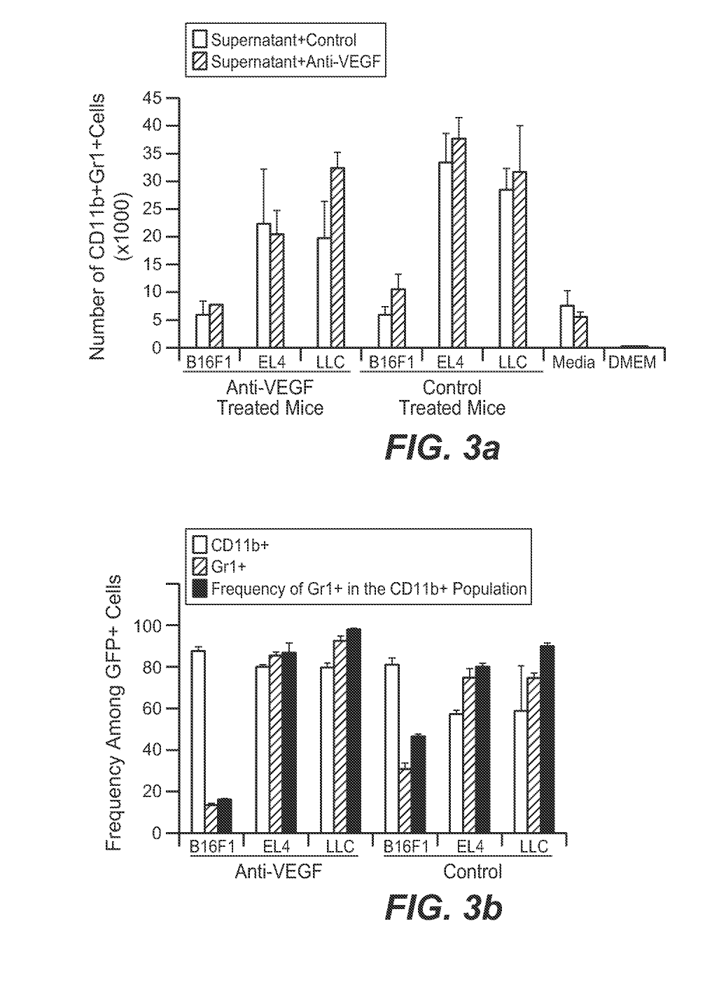

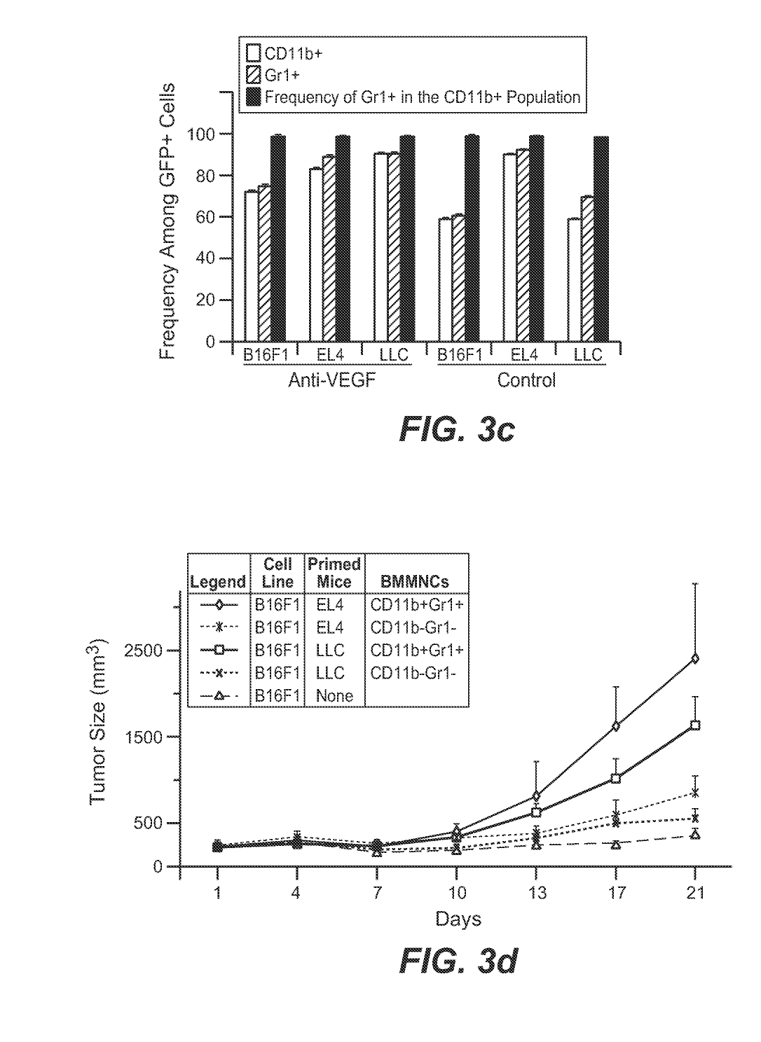

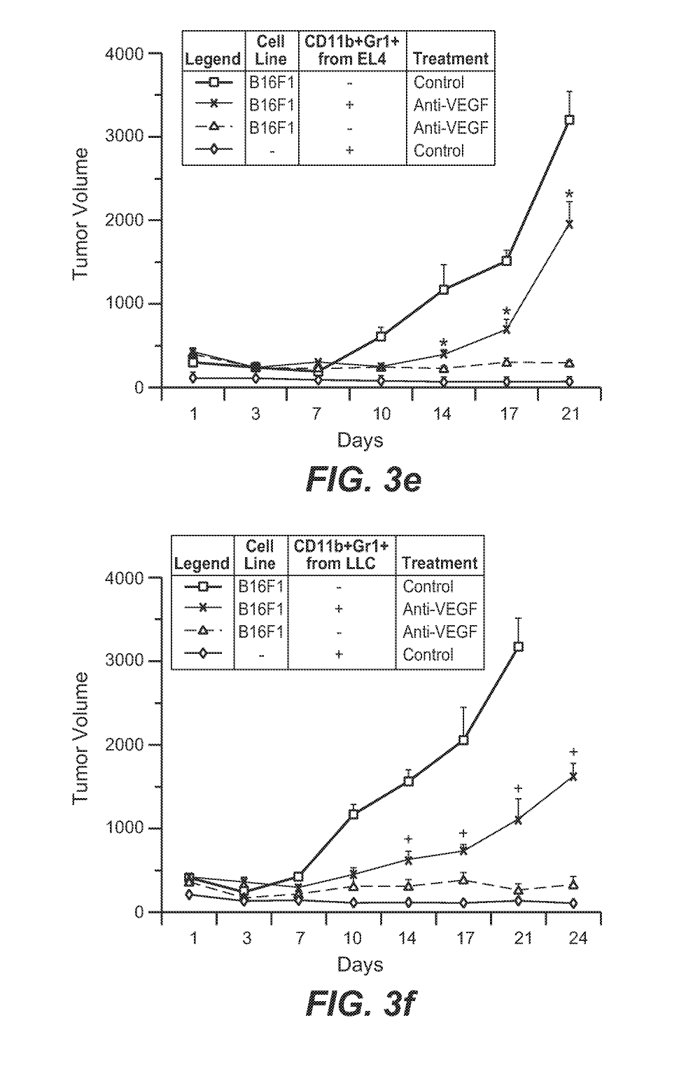

Data shown in FIGS. 3a-d suggest that CD11b+Gr1+ cells, representing the myeloid population, are the major subset of BMMNCs in the development of anti-VEGF resistance. See e.g., Onai, N. et al., Blood, 96:2074-2080 (2000). An in vitro cell migration assay was developed to test BMMNCs exposed to soluble extract harvested from resistant and sensitive tumors. Tumors were grown for 14 days in mice treated with either anti-VEGF or control antibodies. In vitro migration assay indicated greater (p≦0.05) migration capacity of BM CD11b+Gr1+ cells towards the soluble extracts of resistant but not sensitive tumors (FIG. 3a). Therefore, myeloid-chemoattractant factors are present in the soluble extracts of either control- or anti-VEGF-treated tumors, and remained unaffected when anti-VEGF (10 μg/ml) was added to the media. These findings suggest that myeloid cell recruitment is tumor intrinsic, VEGF independent and is not induced by the treatment. These findings are consistent with the data from tumor growth experiments (FIG. 1d) in which anti-VEGF treatment did not effectively block homing of BMMNCs to resistant tumors, and further support the notion that myeloid cell recruitment is intrinsic to tumors as opposed to treatment-induced.

Given the increase in CD11b+Gr1+ cell migration in response to conditioned media from anti-VEGF-resistant tumors (FIG. 3a), FACS analysis was used to study hematopoietic, lymphoid, and myeloid lineages recruited to EL4 and LLC tumors grown in mice. When gated on the CD11b+ subset, EL4 and LLC tumors displayed enrichment for CD11b+Gr1+ cells compared with B16F1 tumors (FIG. 3b). The differences were most pronounced in anti-VEGF treated tumors. In B16F1 tumors, the CD11b+Gr1+ cell population was markedly reduced in anti-VEGF treated, while they remained unaffected in EL4 or LLC tumors (FIG. 3b). In another experiment, the myeloid compartment in mice bearing TIB6, B16F1, EL4 and LLC tumors were analyzed using a FACS machine and monoclonal antibodies against CD11b and Gr1. Flowcytometric analysis of infiltrating BMMNCs in EL4 and LLC tumor isolates displayed a significant (p≦0.05) enrichment for CD11b+Gr1+ cells compared with TIB6 and B16F1 tumors. These results are consistent with the decreased levels of BMMNCs in anti-VEGF-sensitive tumors (FIG. 1d), and provide further support of a correlation between the recruitment of CD11b+Gr1+ cells to tumors and the development of drug resistance. In contrast to the data from CD11b+Gr1+ isolated in tumors, less pronounced changes were found in bone marrow CD11b+ subsets of tumor-bearing mice (FIG. 3c). These data suggest a distinct cross-talk between the bone marrow and tumors in mice bearing resistant tumors as they recruit more CD11b+Gr1+ and also instruct the bone marrow to generate more myeloid cells.

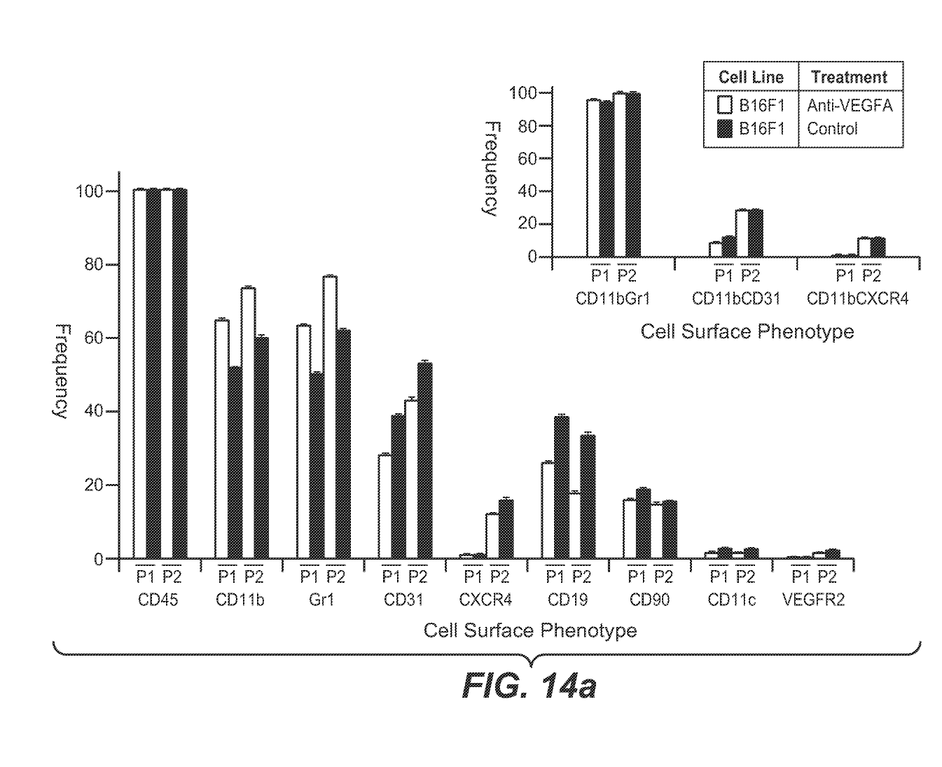

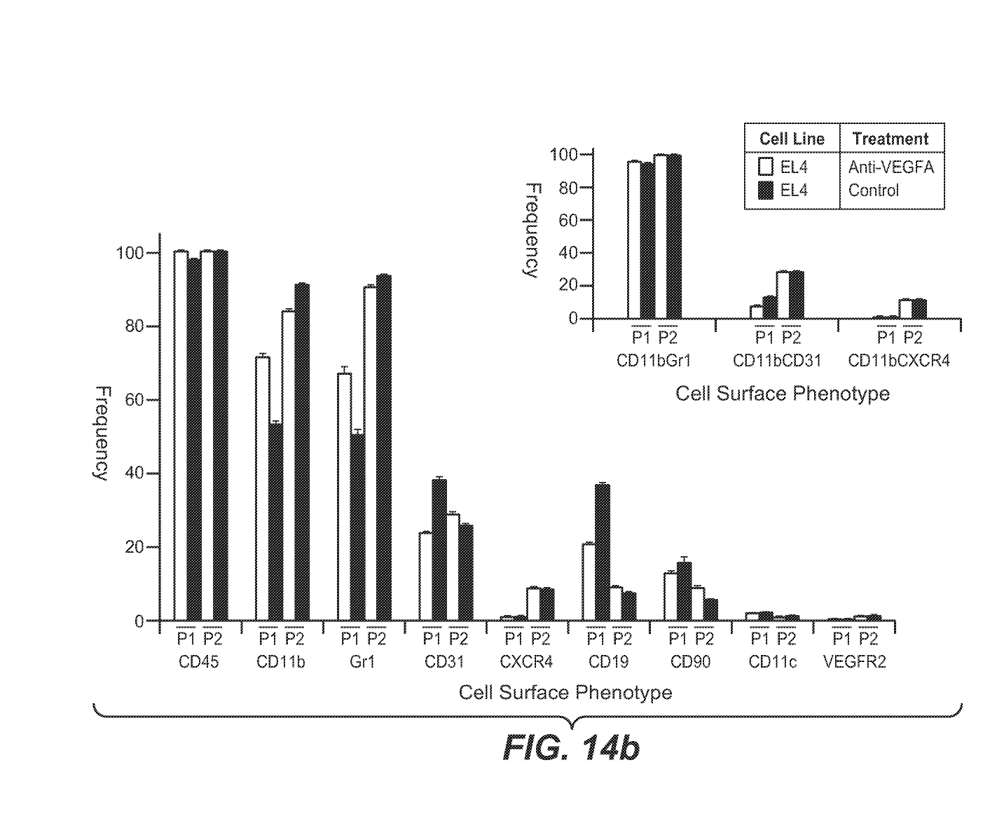

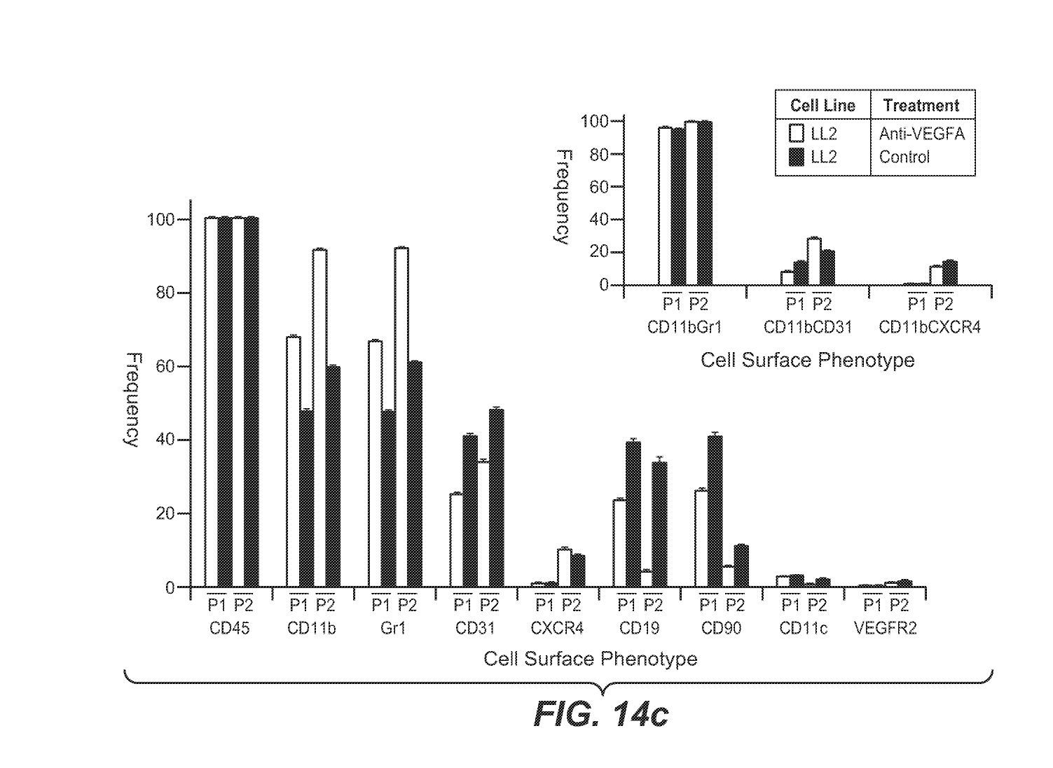

Further analysis of CD11b+Gr1+ cells in resistant and sensitive tumors revealed greater expression of molecules known to be involved in homing and trans-endothelial migration of myeloid cells such as CXCR4 and L-Selectin, respectively. The relative numbers of CD11b+CD31+ (EPCs) and CD11b+CXCR4+ cells (neutrophils), CD19 (B-cells), CD90 (T-cells), CD11c (dendritic cells) and VEGFR-2 in the BMMNCs of tumor bearing mice were similar between treatment groups and tumor types, with the exception of CD19 in some tumors (FIG. 14).

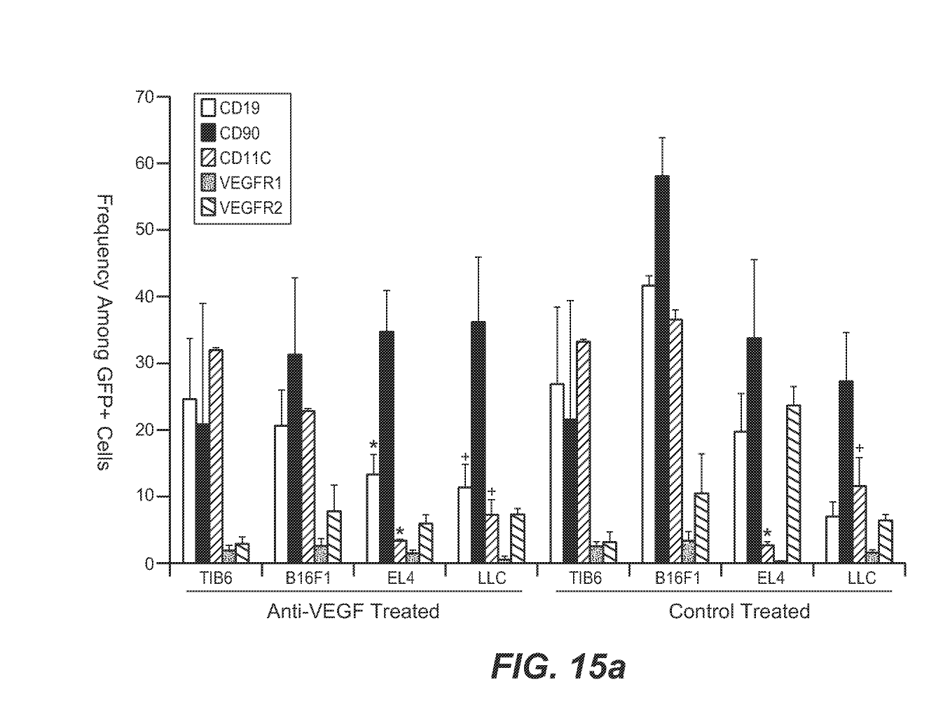

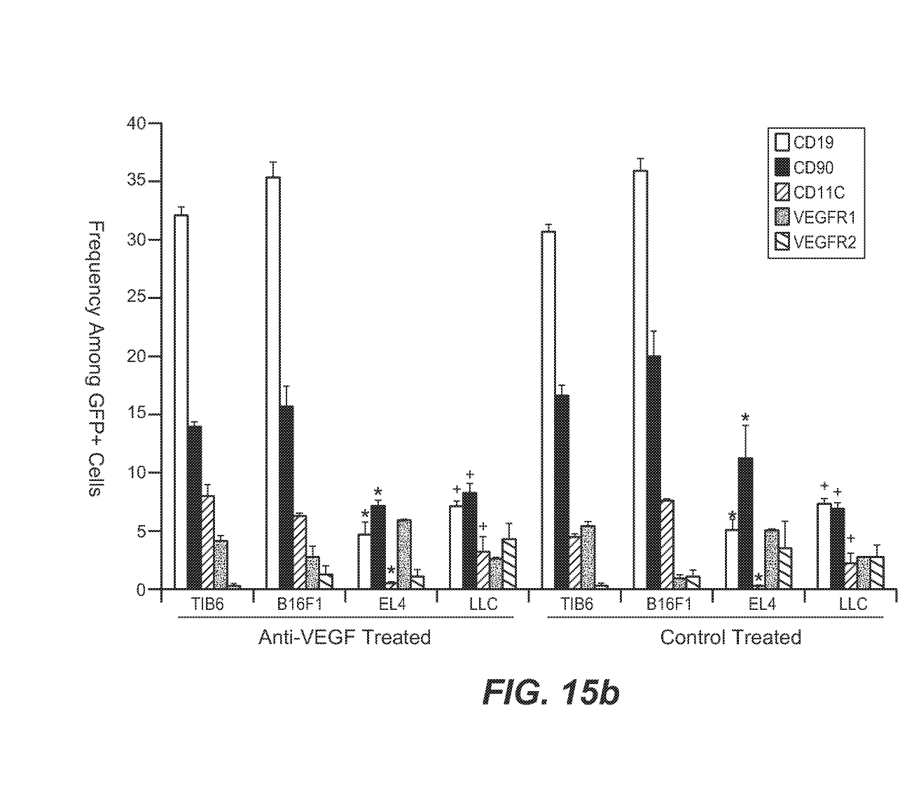

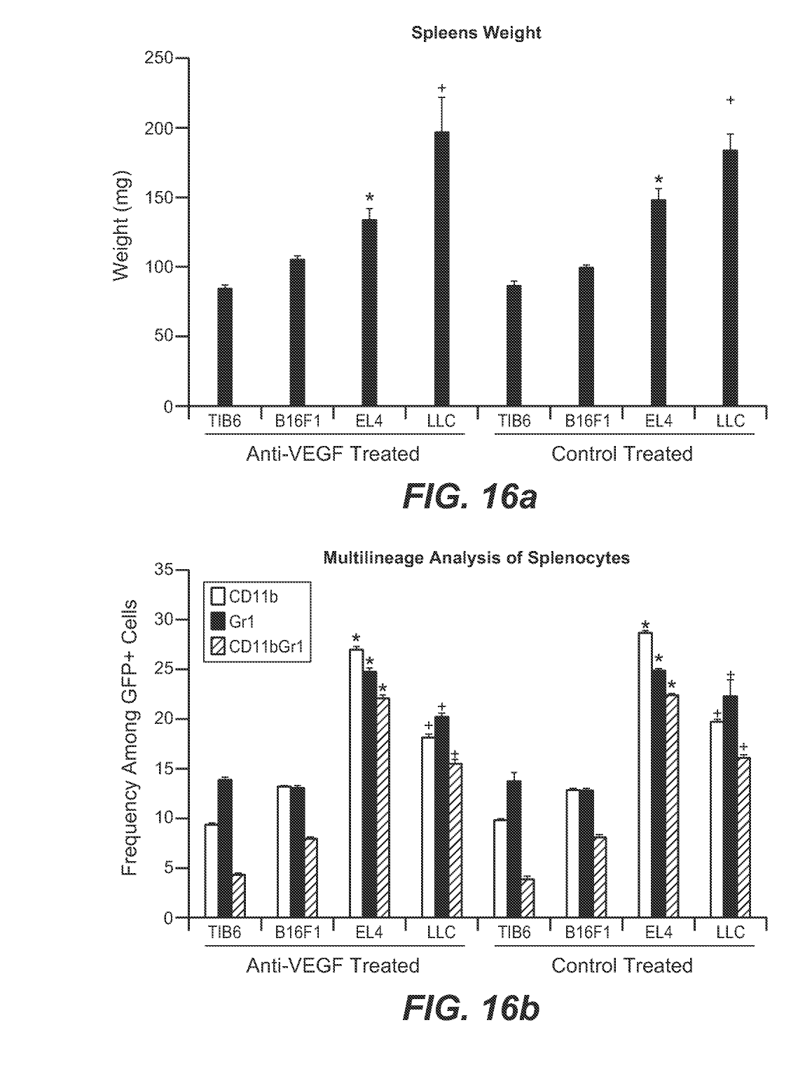

In addition to CD11b and Gr1, expressions of other hematopoietic lineages such as B and T lymphoid, CD11c, and also VEGFR1 and VEGFR2 were investigated in tumor bearing mice (FIG. 15). A significant reduction (p≦0.05) in the frequency of B-lymphoid cells and dendritic cells was notable in resistant tumors (FIG. 15a). In addition, the data indicate a significant difference in the frequency of B- and T-lymphoid as well as dendritic cells in BM of mice bearing resistant tumors compared to the corresponding sensitive ones (FIG. 15b). These observations suggest that the increase in the frequency of myeloid cells in resistant tumors is associated with a reduction in other hematopoietic lineages. In addition to the BM and tumors, spleens in tumor bearing mice were investigated since previous studies suggested that splenic CD11b+Gr1+ cells contribute in tumor expansion. See e.g., Kusmartsev, S. & Gabrilovich, D. I., Cancer Immunol Immunother, 51:293-298 (2002); Bronte, V. et al., Blood, 96:3838-3846 (2000). In support of BM and tumor data, an increase (p≦0.05) in the frequency of CD11b+Gr1+ in spleens and enlarged spleen sizes (p≦0.05) in mice implanted with resistant tumors compared to sensitive ones were found (FIG. 16a and b). Together, these observations suggested a functional role for CD11b+Gr1+ cells as one of the major cell populations in mediating resistance to anti-VEGF treatment.

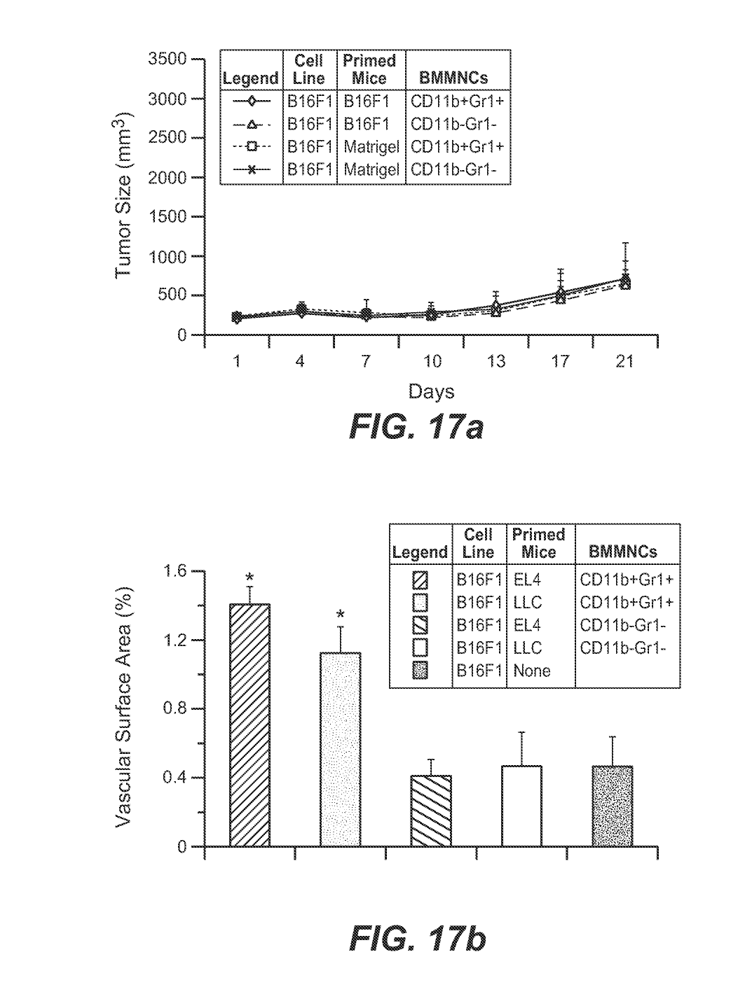

To investigate the functional relevance of myeloid cells in anti-VEGF resistance, CD11b+Gr1+ and CD11b⁻Gr1⁻ subpopulations from the bone morrow of mice primed with EL4 and LLC tumors were isolated (FIG. 17) and admixed them with B16F1 tumor cells.

As shown in FIG. 3d, CD11b+Gr1+ cells were sufficient to mediate resistance to anti-VEGF treatment. However, BMMNCs and tumor-derived GFP+ cells depleted of CD11b+Gr1+ cells failed to mediate resistance. CD11b+Gr1+ cells from the bone marrow of mice primed with anti-VEGF-resistant tumors can mediate resistance to anti-VEGF treatment. Thus, FIG. 3d indicates that CD11b+Gr1+ cells primed by resistant tumors, but not sensitive ones, mediate resistance to anti-VEGF treatment. However, admixture of B16F1 with CD11b+Gr1+ cells isolated from B16F1 or matrigel primed mice did not promote resistance to anti-VEGF treatment compare to CD11b−Gr1− population (FIG. 17a). This further proves the hypothesis that resistant tumors have distinct cross-talk with myeloid compartment compared to sensitive ones. To investigate the impact of CD11b+Gr1+ cells on tumor vasculature, vascular surface area (VSA) in B16F1 admixture with CD11b+Gr1+ and CD11b−Gr1− cells were analyzed (FIG. 17b). These findings indicate that VSA in CD11b+Gr1+ admixture is significantly (p≦0.05) greater than B16F1 alone or admixture with CD11b−Gr1− cells suggesting that development of vasculature is one of the main causes of resistance to anti-VEGF when admixing sensitive cell lines with CD11b+Gr1+ cells. Similar results were obtained when testing tumor associated CD11b+Gr1+ cells isolated from resistant tumors for their ability to confer resistance to sensitive tumors (FIG. 3e, f). Accordingly, both BM- and tumor associated-CD11b+Gr1+ cells are sufficient to confer resistance to anti-VEGF when tested in a cellular-gain-of-function approach.

Anti-VEGF-resistant tumors induce a specific set of genes in bone marrow CD11b+Gr1+ cells

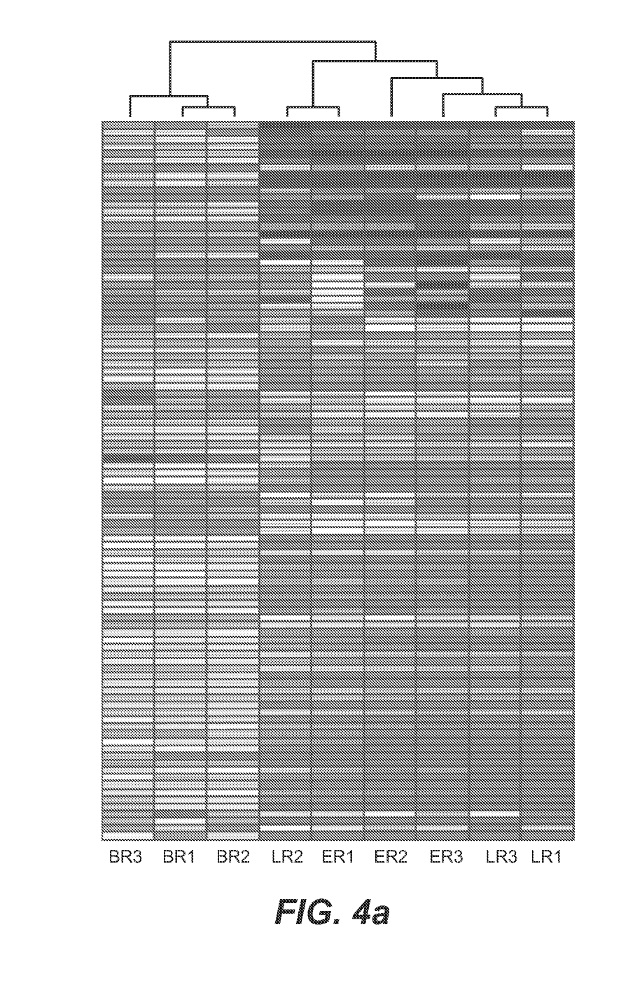

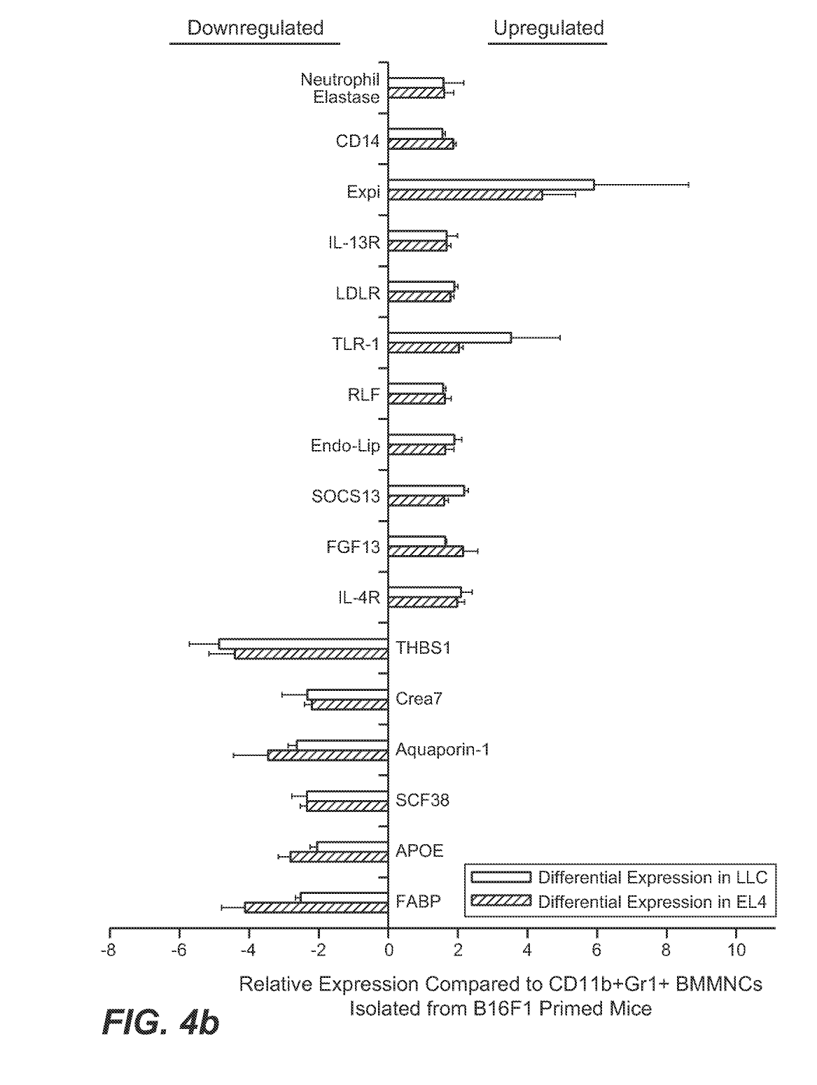

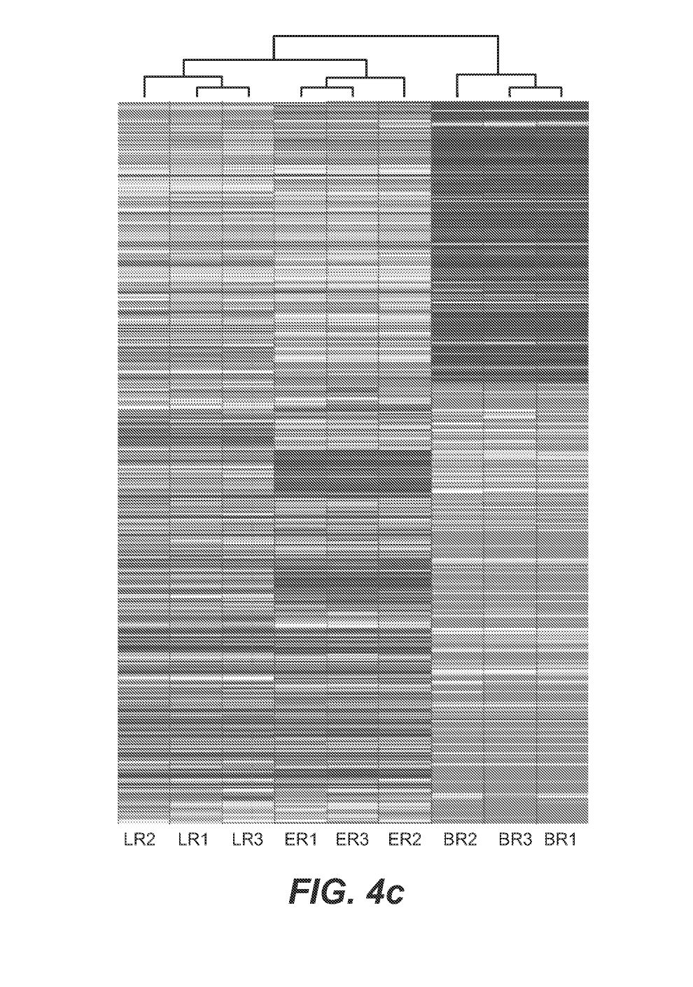

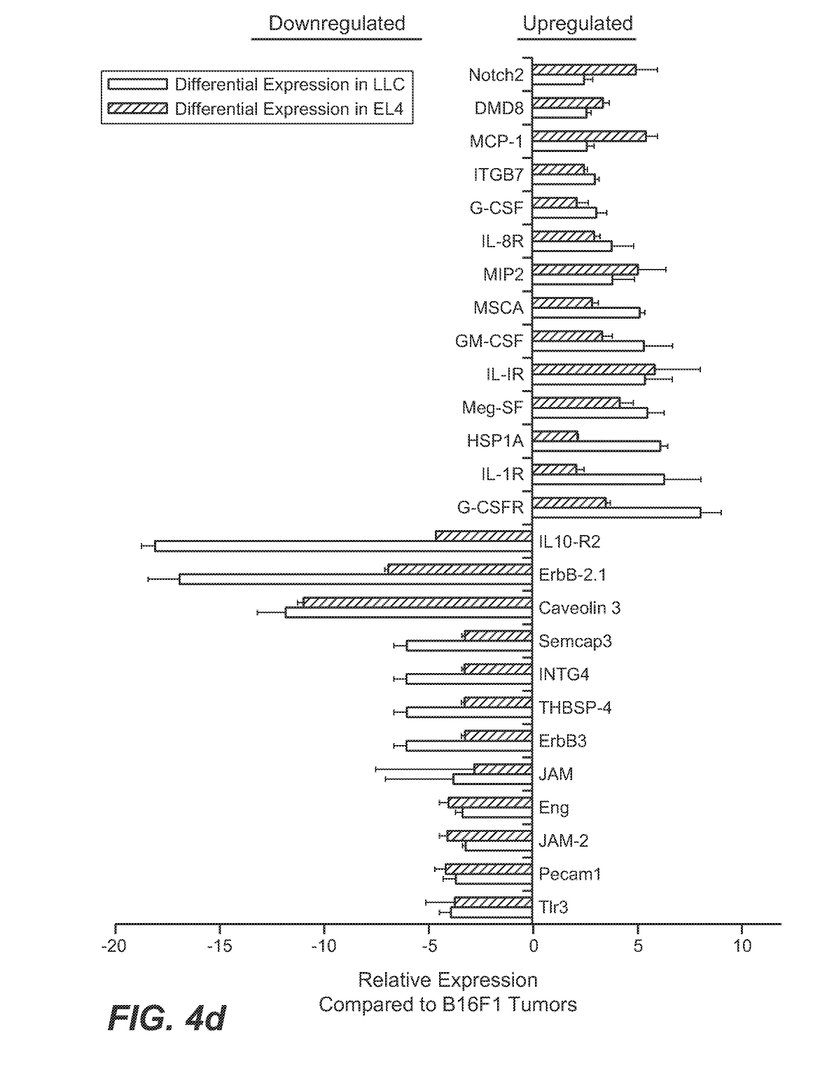

To detect potential differences in the activation status of CD11b+Gr1+ cells in the bone marrow of tumor-bearing mice, gene expression analysis was conducted using DNA arrays. Unsupervised cluster analysis of CD11b+Gr1+ cells primed by anti-VEGF-resistant EL4 or LLC tumors identified a characteristic set of differentially regulated genes, which was distinct from cells primed by anti-VEGF-sensitive B16F1 tumors (FIG. 4a). Gene ontology analysis revealed enrichment of inflammatory cytokines and markers of macrophage/myeloid cell differentiation and alterations in the levels of pro- and anti-angiogenic factors by anti-VEGF-resistant tumors (FIG. 4b). A set of genes commonly upregulated by both anti-VEGF-resistant tumors was identified, of which several are known to be involved in the regulation of angiogenesis, relaxin-like factor (RLF) (see, e.g. Silvertown, J. D., Summerlee, A. J. & Klonisch, T. Relaxin-like peptides in cancer. Int J Cancer 107:513-9 (2003)), and phospholipid scramblase (Endo-Lip) (see, e.g. Favre, C. J. et al. Expression of genes involved in vascular development and angiogenesis in endothelial cells of adult lung. Am J Physiol Heart Circ Physiol 285:H1917-38 (2003)).

Another category of genes associated with differentiation and/or activation of myeloid cells was prominently upregulated in CD11b+Gr1+ cells by anti-VEGF-resistant tumors, including the receptors for IL-4 (see, e.g. Palmer-Crocker, R. L., Hughes, C. C. & Pober, J. S. IL-4 and IL-13 activate the JAK2 tyrosine kinase and Stat6 in cultured human vascular endothelial cells through a common pathway that does not involve the gamma c chain. J Clin Invest 98:604-9 (1996)) and IL-13 (see, e.g. Roy, B. et al. IL-13 signal transduction in human monocytes: phosphorylation of receptor components, association with Jaks, and phosphorylation/activation of Stats. J Leukoc Biol 72:580-9 (2002)), CD14 (see, e.g. Scott, C. S. et al. Flow cytometric analysis of membrane CD11b, CD11c and CD14 expression in acute myeloid leukaemia: relationships with monocytic subtypes and the concept of relative antigen expression. Eur J Haematol 44:24-9 (1990)), TLR-1 (see, e.g., Edfeldt, K., Swedenborg, J., Hansson, G. K. & Yan, Z. Q. Expression of toll-like receptors in human atherosclerotic lesions: a possible pathway for plaque activation. Circulation 105:1158-61 (2002)) (FIG. 4). Conversely, thrombospondin-1, a potent angiogenesis inhibitor (see, e.g., Good, D. et al. A tumor suppressor-dependent inhibitor of angiogenesis is immunologically and functionally indistinguishable from a fragment of thrombospondin. Proc. Nat. Acad. Sci. USA 87:6624-6628 (1990); and, Iruela-Arispe, M. L., Bornstein, P. & Sage, H. Thrombospondin exerts an antiangiogenic effect on cord formation by endothelial cells in vitro. Proc Natl Acad Sci USA 88:5026-30 (1991)), was among the genes significantly downregulated by both anti-VEGF-resistant tumors.

In yet another microarray experiment showed that resistant tumors represent a distinct profile of gene expression. Gene tree analysis of CD11b+Gr1+ cells isolated from the bone marrow of mice implanted with EL4 (E1-3), LLC (L1-3), B16F1 (B1-3) and TIB6 (T1-3) tumors and treated with anti-VEGF was done. Genes down-regulated, unchanged and up-regulated were identified. A characteristic set of changes induced by anti-VEGF-resistant tumors, which is distinct from that induced by anti-VEGF-sensitive tumors, were identified. Array analysis of differentially expressed genes in bone marrow CD11b+Gr1+ cells isolated from mice bearing TIB6, B16F1, EL4 and LLC tumors and treated with anti-VEGF for 17 days was performed. Genes potentially involved in the regulation of angiogenesis or myeloid cell differentiation and migration, with significant changes (p≦0.05,>1.5 fold) in expression levels in resistant versus sensitive tumors were identified. Upregulated genes known to be involved in the regulation of angiogenesis included interleukin-11 receptor (IL-11R), interleukin-1 receptor II (IL-1RII), interferon transmembrane 1 (IFN TM1), tumor necrosis factor receptor superfamily member 18 (TNFRSF18), Wingless integration 5A (WNT5A), secretory carrier membrane 1, heat shock protein (HSP86), epidermal growth factor receptor (EGFR), Eph receptor B2 (EphRB2), G-protein coupled receptor 25 (GPCR25), hepatoma derived growth factor (HGF), angiopoietin like-6, ephrin receptor RA7 (Eph-RA7), semaphorin V1b, neurotrophin 5, claudin-18, metalloprotease-disintegrin MDC15 (MDC15), extra cellular matrix (ECM) and a disintegrin and metalloprotease with thrombospondin motif 7B (ADAMTS7B). Genes that were down-regulated included neuronal cell adhesion molecule (NCAM-140), fibronectin type III, Wiskott-Aldrich syndrome protein interacting protein (WIP), CD74, intercellular adhesion molecule 2 (ICAM-2), Jagged1, integrin alpha-4 (Itga4), integrin BETA-7 (ITGB7), transforming growth factor-beta type II receptor (TGF-BII-R), TGFb inducible early protein (TGFb IEP), mothers against decapentaplegic (MAD) and the C. elegans protein SMA-4 (Smad4), bone morphogenetic protein receptor 1A (BMPR1A), CD83, Dectin-1, CD48, E-selectin, interleukin-15 (IL-15), suppressor of cytokine signaling 4, cytokine receptor related protein 4 (Cytor4) and chemokine (C—X3-C) receptor 1 (CX3CR1).

A set of genes commonly upregulated by both resistant tumors was identified, of which several are known to be involved in the regulation of angiogenesis, including relaxin-like factor (RLF) (Ho, R. L. et al. Immunological responses critical to the therapeutic effects of adriamycin plus interleukin 2 in C57BL/6 mice bearing syngeneic EL4 lymphoma, Oncol Res, 5:363-372 (1993)), Neurotrophin 5 (Lazarovici, P. et al., Nerve growth factor (NGF) promotes angiogenesis in the quail chorioallantoic membrane, Endothelium, 13:51-59 (2006)), phospholipid scramblase (Endo-Lip) (Favre, C. J. et al., Expression of genes involved in vascular development and angiogenesis in endothelial cells of adult lung, Am J Physiol Heart Circ Physiol, 285:H1917-1938 (2003)), Angiopoietin like-6, Semaphorin VIb, Eph RA7, Eph RB2 and FGF13. Furthermore, GM-CSF (Rapoport, A. P. et al., Granulocyte-macrophage colony-stimulating factor (GM-CSF) and granulocyte colony-stimulating factor (G-CSF): receptor biology, signal transduction, and neutrophil activation, Blood Rev, 6:43-57 (1992)) that is associated with differentiation and/or activation of myeloid cells was also upregulated in CD11b+Gr1+ BM cells isolated from mice bearing resistant tumors. Several genes known to be involved in the activation/generation of dendritic cells are completely downregulated in BM CD11b+Gr1+ isolated from resistant tumors. This includes, CD83, CD48, Crea7 and Dectin-1 (see e.g., Lechmann, M et al., CD83 on dendritic cells: more than just a marker for maturation, Trends Immunol 23:273-275 (2002)), IL-15 (see e.g., Feau, S. et al., Dendritic cell-derived IL-2 production is regulated by IL-15 in humans and in mice, Blood 105:697-702 (2005)). and CX3CR1 (see e.g. Niess, J. H. et al., CX3CR1-mediated dendritic cell access to the intestinal lumen and bacterial clearance, Science 307:254-258 (2005)). The molecular data is in line with multilineage analysis of BMMNCs (FIG. 15) where there is a significant (p≦0.05) reduction in the frequency of CD11c+ cells both in the BM and tumors in mice bearing resistant tumors. In addition, several members of TGF-beta superfamily (see e.g., Derynck, R et al., TGF-beta signaling in tumor suppression and cancer progression, Nat Genet 29:117-129 (2001)) including Smad4 and BMPR1A are among downregulated genes suggesting a role for TGF-beta pathway in regulating activation/differentiation of CD11b+Gr1+ cells in mice bearing resistant tumors.

In addition, gene expression analysis from LL2, EL4 and B16F1 tumors was conducted and analyzed for gene specifically up or down-regulated in anti-VEGF treated resistant (EL4+LL2), but not in sensitive (B16F1) tumors. Overall gene-expression patterns were distinct between all tumor types. As shown in FIG. 4d, many of the genes whose expression was altered between anti-VEGF resistant and sensitive tumors belong to the class of chemokines and cytokines, suggesting the presence of inflammatory cells in anti-VEGF resistant tumors. In addition, various pro- or anti-angiogenic factors were identified.

Similarly, additional gene expression analysis in anti-VEGF treated TIB6, B16F1, EL4 and LLC tumors indicated a distinct profile of gene-expression among all tumor types. Upregulated genes included insulin-like growth factor 2, binding protein 3 (IGF2BP3), Heat shock protein 9A (HSP9A), Fibroblast growth factor 18 (FGF18), connective tissue growth factor related protein WISP-1 (ELM1), lens epithelium-derived growth factor a (Ledgfa), scavenger receptor type A, Macrophage C-type lectin, polymeric immunoglobulin receptor 3 precursor (Pigr3), Macrophage scavenger receptor type I (Macrophage SRT-1), G protein-coupled receptor, small inducible cytokine A7 (ScyA7), Interleukin-1 Receptor2 (IL-1R2), Interleukin-1 inducible protein (IL-1 inducible protein), Interleukin-1 beta (IL-1beta), LIX (LPS-induced CXC chemokine (Scyb5) gene|chemokine (C—X—C motif) ligand 5). Genes that were down-regulated included transforming growth factor beta (TGF-B), Frizzled (FIZZ1), Wolfram syndrome 1 homolog (Wfs1), transmembrane protein 14A (TP 14A), extracellular matrix associated protein (EMAP), sulfatase 2 (SULF-2), extracellular matrix 2, connective tissue growth factor (CTFG), tissue factor pathway inhibitor (TFPI), resistin like-molecule alpha mRNA|strain C57BL/6 XCP2 protein (Xcp2) gene (XCP2), receptor activity modifying protein 2 (Ramp2), RAR-related orphan receptor alpha (ROR-alpha), ephrin B1, secreted protein acidic and rich in cysteine-like 1 (SPARC-like 1), Semaphorin A. Analysis of differentially expressed genes (more than 2 fold, p≦0.05) in resistant versus sensitive (TIB6+B16F1) tumors identified several cytokines known to be involved in the mobilization of BMMNCs to the peripheral blood including granulocyte colony stimulating factor (G-CSF) (see e.g., Rapoport, A. P. et al., Granulocyte-macrophage colony-stimulating factor (GM-CSF) and granulocyte colony-stimulating factor (G-CSF): receptor biology, signal transduction, and neutrophil activation, Blood Rev 6:43-57 (1992)), and monocyte chemoattractant protein (MCP-1) (see e.g., Leonard, E. J. et al., Secretion of monocyte chemoattractant protein-1 (MCP-1) by human mononuclear phagocytes, Adv Exp Med Biol 351:55-64 (1993)). Furthermore, factors involved in inflammation such as macrophage inflammatory protein (MIP-2) (see e.g. Cook, D. N., The role of MIP-1 alpha in inflammation and hematopoiesis, J Leukoc Biol, 59:61-66 (1996)) and IL-1R (see e.g., Dinarello, C. A., Blocking IL-1 in systemic inflammation, J Exp Med, 201:1355-1359 (2005) were among differentially expressed genes. A majority of the above cytokines, such as G-CSF are also known to be involved in differentiation (see e.g., McNiece, I. K. et al., Recombinant human stem cell factor synergises with GM-CSF, G-CSF, IL-3 and epo to stimulate human progenitor cells of the myeloid and erythroid lineages, Exp Hematol, 19:226-231 (1991)) and proliferation (see e.g., Lemoli, R. M. et al., Proliferative response of human acute myeloid leukemia cells and normal marrow enriched progenitor cells to human recombinant growth factors IL-3 GM-CSF and G-CSF alone and in combination, Leukemia, 5:386-391 (1991)) of hematopoietic progenitors to myeloid cells. Therefore, in addition to priming and promoting mobilization of hematopoietic cells to the periphery, mice bearing resistant tumors may share the ability to stimulate myeloid cell differentiation.

These findings support the conclusion from gene expression studies in CD11b+, Gr1+ cells, and suggest that differential regulation of pro- or anti-angiogenic activities and inflammatory cytokines and chemokines by anti-VEGF resistant tumors may potentially contribute to resistance of anti-VEGF-resistant tumors.

Combining Anti-VEGF with Agents Interfering with Myeloid Cell Functions Suppresses Tumor Angiogenesis and Growth.

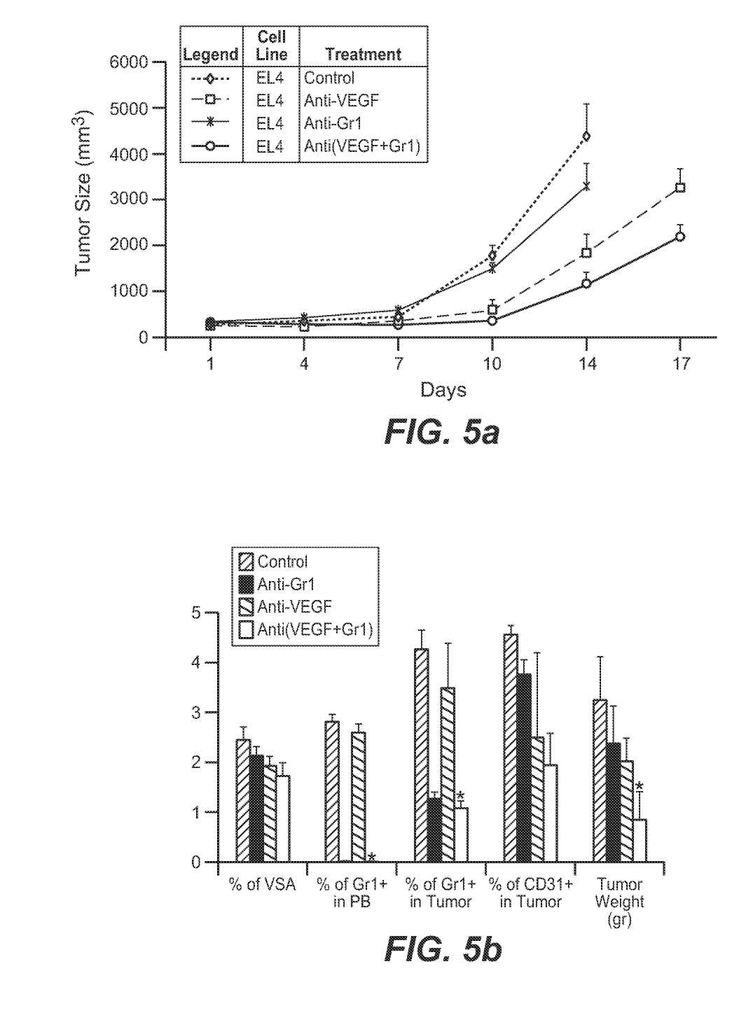

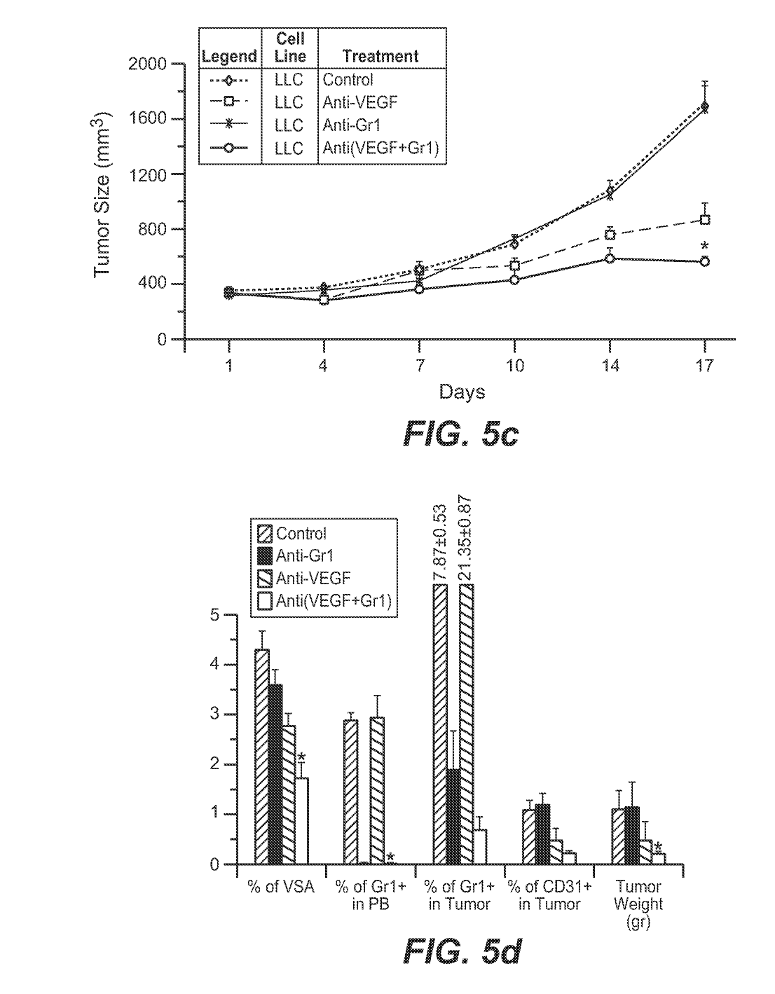

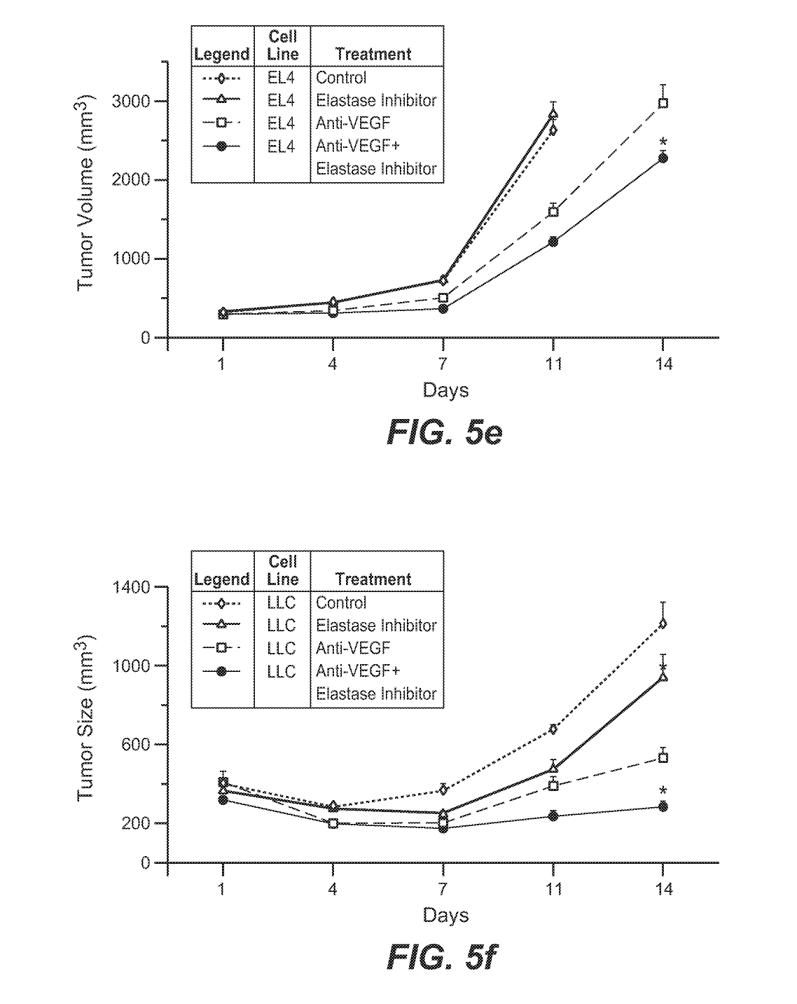

An anti-GR1 antibody reducing the numbers of Gr1+ myeloid cells in the peripheral circulation was tested alone or in combination with anti-VEGF in the context of EL4 (FIG. 5a-b) and LL2 tumors (FIG. 5c-d). When administered alone, anti-GR1 treatment was effective in reducing the numbers of peripheral and tumoral Gr1+ cells, however, it did not affect tumor growth and vascularization of EL4 tumors significantly (FIG. 5a-b). However, when the anti-GR1 antibody was combined with G6-23, we observed a trend towards prolonged tumor growth delay and onset of tumor resistance of either EL4 (FIG. 5b) or LL2 (FIG. 5d) tumors when compared to the effects induce by anti-VEGF-A treatment alone was present in the combination treatment groups. Histological analysis of LL2 tumors revealed a trend towards a reduction in Gr1+ myeloid cells by FACS and vascular surface areas (VSA), which correlated with a reduction in tumor growth rates (FIG. 5c and d) in the combination treatment group.

The gene expression analysis revealed a significant increase in neutrophil elastase expression in the tumor and CD11b, Gr1+ bone marrow cells by anti-VEGF-resistant tumor cell lines (FIG. 4b). Elastase produced by neutrophils was described to promote tumor cell proliferation, motility and to stimulate growth of various tumor types. See, e.g., Sun, Z. & Yang, P. Role of imbalance between neutrophil elastase and alpha 1-antitrypsin in cancer development and progression. Lancet Oncol 5:182-90 (2004). In addition, a role for neutrophil elastase in the regulation of neutrophil mobilization and angiogenesis was proposed. See, e.g., Shamamian, P. et al. Activation of progelatinase A (MMP-2) by neutrophil elastase, cathepsin G, and proteinase-3: a role for inflammatory cells in tumor invasion and angiogenesis. J Cell Physiol 189:197-206 (2001). An anti-VEGF treatment was combined with an elastase inhibitor. Combination treatment resulted in a significant reduction of tumor volumes and terminal tumor weights of LLC and EL4 tumors (FIG. 5e and f). Similar to treatment with anti-GR1 antibody (FIG. 5a-d), the elastase inhibitor induced almost complete ablated circulatiory myeloid cells, however, within the tumors, a 2 to 3 fold reduction when compared to control treatment was found. Based on this, we hypothesize that certain myeloid progenitor cells that lack CD11b or GR1 expression may not be affected by treatment. Alternatively, progenitor cells may potentially infiltrate tumors and differentiate to myeloid cells in situ. Strategies inducing more profound myeloid cell ablation within anti-VEGF treated tumors may further increase the therapeutic effects of the combination treatment. Combined, these finding suggest improved therapeutic efficacy when combining anti-VEGF with compounds targeting myeloid cell functions and provide the first evidence that pro-angiogenic functions of myeloid cells may contribute to the development of resistance towards anti-VEGF treatment. Furthermore, these findings support the notion that several pathways may be involved in the recruitment and activation of myeloid cells to anti-VEGF resistant tumors.

Phenotypic Characteristics of CD11b+GR1+ in Resistant Tumors

Based on the distinct functional characteristics of CD11b+Gr1+ cells in resistant tumors, their cellular properties were investiged. The expression of molecules known to be involved in the mobilization (CXCR4 (see e.g., Orimo, A. et al., Stromal fibroblasts present in invasive human breast carcinomas promote tumor growth and angiogenesis through elevated SDF-1/CXCL12 secretion, Cell, 121:335-348 (2005))) and transendothelial migration (L-Selectin (see e.g., Simon, S. I. et al., L-selectin (CD62L) cross-linking signals neutrophil adhesive functions via the Mac-1 (CD11b/CD18) beta 2-integrin, J Immunol, 155:1502-1514 (1995))) of hematopoietic cells were examined. In addition, TAMs, known by the expression of F480, have been described as a subset of myeloid cells with the potential to increase tumor growth (see e.g., Luo, Y. et al., Targeting tumor-associated macrophages as a novel strategy against breast cancer, J Clin Invest, 116:2132-2141 (2006)). Depletion of TAMs, using clodronate, improved the efficacy of anti-VEGF treatment in mice bearing resistant tumors. Also, Tie2 positive TAMs were found to localize within tumor vessels and to mediate angiogenesis (see e.g., De Palma, M. et al., Tie2 identifies a hematopoietic lineage of proangiogenic monocytes required for tumor vessel formation and a mesenchymal population of pericyte progenitors, Cancer Cell, 8:211-226 (2005)). Therefore, the expression of CXCR4, L-Selectin, F4/80 and Tie-2 in myeloid fraction in anti-VEGF-treated -resistant and -sensitive tumors were investigated.

C57BL/6 mice (n=5) were implanted with TIB6, B16F1, EL4 and LLC tumors and were treated with anti-VEGF or control antibodies as described. Tumor isolate from each mouse was harvested after 17 days and was stained with antibodies against CD11b, Gr1, CXCR4, F480, L-Selectin and Tie2. A significant difference (p≦0.05) in the expression of CXCR4, F480, L-Selectin and Tie2 subsets was observed when comparing tumor associated CD11b+Gr1+ in mice bearing resistant tumors versus corresponding sensitive ones. BMMNCs were isolated from tumor bearing mice and were stained with the same markers as described above. Consistent with tumor analysis, there was a significant difference (p≦0.05) in the frequency of CXCR4, F480, L-Selectin and Tie2 subsets in the GFP+CD11b+Gr1+ cells in resistant versus corresponding population in sensitive tumors.

Flowcytometric analysis revealed that tumor associated CD11b+Gr1+ in resistant tumors are highly enriched (p≦0.05) for the expression of CXCR4, F480, L-Selectin and Tie2. A similar picture was obtained when BM CD11b+Gr1+ cells isolated from tumor bearing mice were analyzed. These observations suggest that CD11b+Gr1+ in resistant tumors are more potent in mobilization, transendothelial migration and homing to the tumors.

Distinct Mechanisms Govern Resistance to Anti-VEGF and Chemotherapeutic Agents

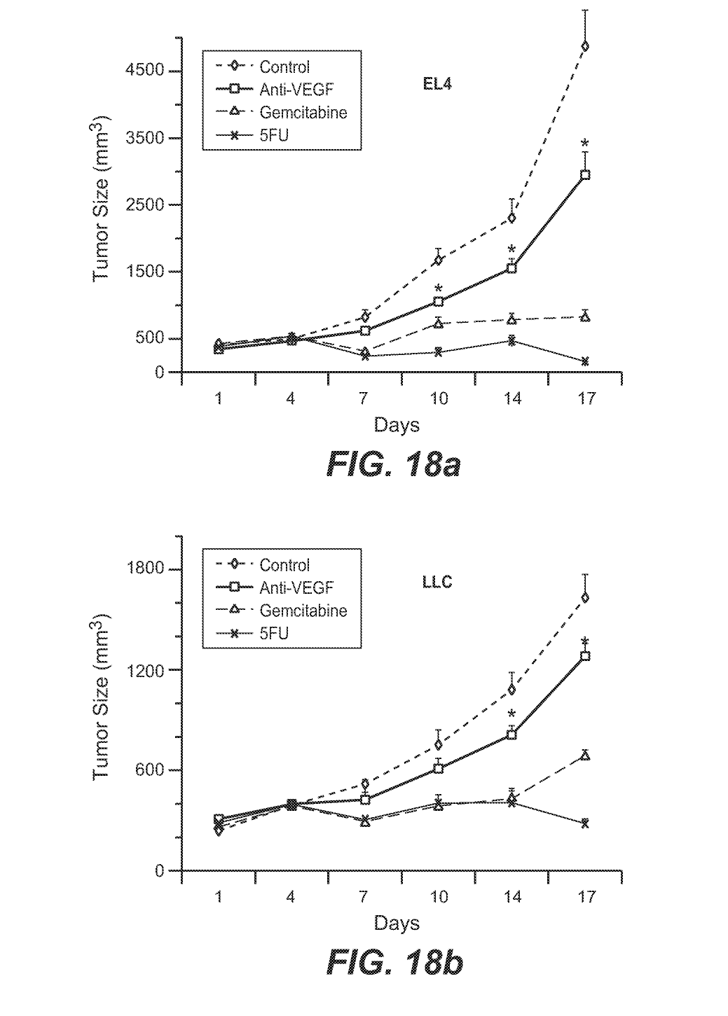

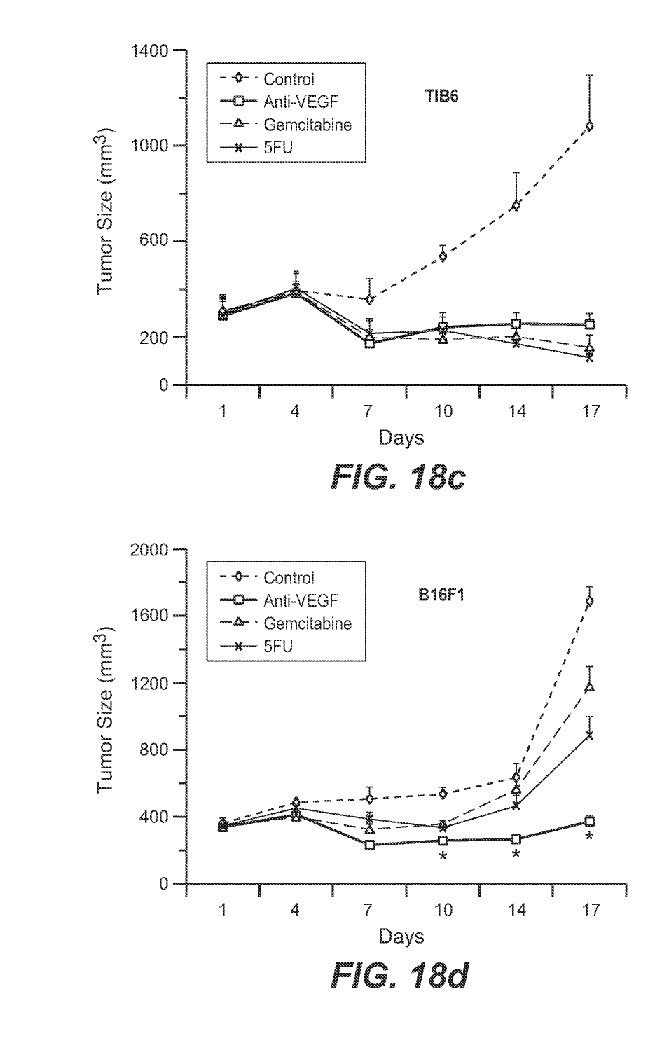

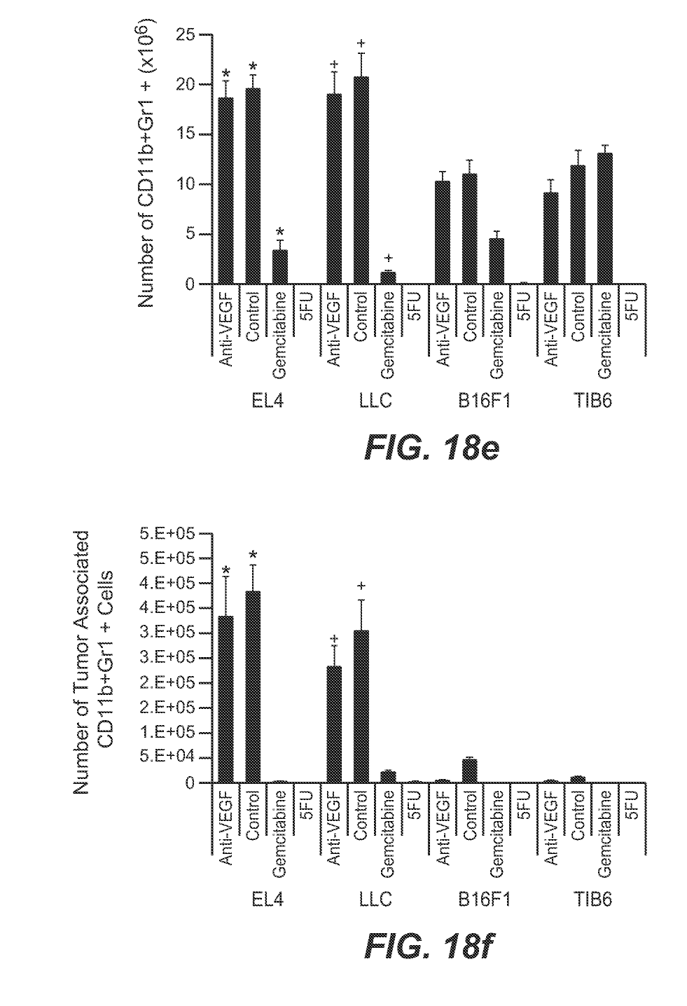

Understanding cellular mechanisms of resistance to anti-VEGF raises the question whether myeloid cells also mediate resistance to other anti-cancer compounds. Therefore, we investigated tumor resistance to two commonly used chemotherapeutic agents including 5-Fluorouracil (5FU) and Gemcitabine (see e.g., Pasetto, L. M. et al., Old and new drugs in systemic therapy of pancreatic cancer, Crit Rev Oncol Hematol, 49:135-151 (2004)). Anti-VEGF resistant and sensitive tumors displayed different responses to chemotherapy. As shown in FIG. 18a and b, both anti-VEGF resistant tumors, i.e. EL4 and LLC, showed a complete response to 5FU and a partial resistance to Gemcitabine at later time point that is much slighter than resistance to anti-VEGF treatment. In anti-VEGF sensitive cell lines, TIB6 tumors were found to be completely sensitive to both compounds with no significant difference compared to response to anti-VEGF treatment (FIG. 18c). However, B16F1 tumors showed resistance to both 5FU and Gemcitabine compared to anti-VEGF treatment (FIG. 18d). Therefore, the data clearly indicate that the profile of resistance to anti-VEGF does not correspond to chemotherapy in resistant and sensitive tumors and suggest that different mechanisms are involved in development of resistance in an antiangiogenic approach vs. chemotherapeutic agents. Analysis of BM cells showed complete exhaustion of CD11b+Gr1+ cells in all of the 5FU treated mice and to a lesser degree in Gemcitabine treated animals (FIG. 6e). However, lack of CD11b+Gr1+ cells in B16F1 tumors treated with Gemcitabine or 5FU (FIG. 6f) minimizes the involvement of myeloid cells in development of resistance to chemotherapy.

Recruitment of CD11b+Gr1+ cells to primary tumors represents a cellular mechanism mediating resistance to anti-VEGF treatment within a subset of experimental tumors in mice. Gene expression profiling enabled identification of a set of genes that are differentially regulated in CD11b+Gr1+ cells in the bone marrow of mice bearing anti-VEGF-resistant tumors compared with anti-VEGF-sensitive tumors. Among them, several pro- or anti-angiogenic factors and markers of myeloid cell activation that became upregulated during tumor priming were found. Recruitment of myeloid cells to tumors is involved in the development of drug resistance and represents one of the earliest steps in the cascade of events. Compounds targeting tumor-derived factors regulating recruitment and/or activation of myeloid cells can be combined with anti-angiogenic compounds. Selective blockade of tumor-derived chemo-attractants for myeloid cells may be advantageous when compared with a systemic myeloid cell ablation strategy, e.g., to avoid potential complications of prolonged systemic suppression of parts of the innate immune system (see, e.g. Lewis, C. E. & Pollard, J. W. Distinct role of macrophages in different tumor microenvironments. Cancer Research 66:605-612 (2006)). Antagonists of pro-angiogenic factors secreted by tumor-infiltrating myeloid cells can be used in combination treatment with anti-VEGF compounds. Targeting factors that regulate specific functions of myeloid cells may indirectly affect tumor angiogenesis and reduce tumor resistance to anti-VEGF therapy. See FIG. 5.

Clinical evaluation of the anti-VEGF monoclonal antibody bevacizumab has shown significant single-agent activity in various human cancers, including renal and ovarian carcinomas (see, e.g., Ferrara, N., Hillan, K. J., Gerber, H. P. & Novotny, W. Discovery and development of bevacizumab, an anti-VEGF antibody for treating cancer. Nat Rev Drug Discov 3:391-400 (2004); and, Jain, R. K., Duda, D. G., Clark, J. W. & Loeffler, J. S. Lessons from phase III clinical trials on anti-VEGF therapy for cancer. Nat Clin Pract Oncol 3:24-40 (2006)). During the broad clinical development of bevacizumab in most human tumor types, it became apparent that in many tumors, the robust therapeutic effects were obtained in combination with chemotherapeutic agents. The nature of the underlying molecular and cellular events leading to the increased therapeutic benefits in combination treatment with cytotoxic compounds are under examination. It has been proposed that increased tumor drug uptake as a consequence of vessel normalization (reviewed in Jain et al. in Jain, R. K. Normalizing tumor vasculature with anti-angiogenic therapy: a new paradigm for combination therapy. Nat Med 7:987-9 (2001)) and/or interference with endothelial cell recovery following cytotoxic damage of the tumor vasculature (reviewed in Ferrara et al. in Ferrara, N., Hillan, K. J., Gerber, H. P. & Novotny, W. Discovery and development of bevacizumab, an anti-VEGF antibody for treating cancer. Nat Rev Drug Discov 3:391-400 (2004)) may account for the increased therapeutic benefit (reviewed in Ferrara & Kerbel in Ferrara, N. & Kerbel, R. S., Angiogenesis as a therapeutic target. Nature 438:967-74 (2005)). Without being bound to a single theory, the identification of a role for myeloid cells in the mechanism leading to resistance to anti-VEGF treatment provides further support for the notion that the myelosuppressive effects associated with the majority of cytotoxic compounds may contribute to the increased tumor growth inhibition. It was observed that the reduction in myeloid cell numbers within primary lung tumors in patients treated with chemotherapy correlated with survival (see, e.g. Di Maio, M. et al. Chemotherapy-induced neutropenia and treatment efficacy in advanced non-small-cell lung cancer: a pooled analysis of three randomised trials. Lancet Oncol 6:669-77 (2005)).

DNA array analysis of CD11b+Gr1+ cells identified changes in gene expression, which are distinct between anti-VEGF-resistant and -sensitive tumors, demonstrating a remarkable crosstalk between tumors grown in the flank of mice and a subset of cells in the bone marrow (FIG. 4a).

### Example 2

### Additional Factors From Tumor Models which are Resistant to Anti-VEGF Treatment

Additional factors from tumors which may directly or indirectly aid or provide resistant to tumors, were identified. Mouse lymphoma tumor lysates that are resistant to anti-VEGF treatment (e.g., EL4 and L1210) were treated with anti-VEGF antibody (G6-31) at 5 mg/kg/week, twice/week for 2 weeks. After treatment, the tumors were pooled and homogenized in 6 ml RIPA buffer 2× with protease inhibitors (Roche). The homogenization was centrifuged 2×15 minutes at 14,000 rpm in eppendorf centrifuge. The supernatant was diluted 1:1 in 20 mM Tris, pH 7.5, 50 mM NaCl and applied to 1 ml HiTrap HS. The column was washed with start buffer (20 mM Tris, pH 7.5, 50 mM NaCl) and then eluted with about 5 column volumes of step wise increase in NaCl concentrations (0.25M NaCl, 0.5 M NaCl, 1 M NaCl, and 3 M NaCl). Peak fractions at each step were collected. See FIG. 8.

A variety of factors were found in the high salt fractions from EL4 and L1210 that contribute to chemotactic activity (e.g., by a monocyte migration assay) or a proliferation assay (e.g., a HUVEC proliferation assay). For example, bFGF was found in the high salt fraction and was shown to contribute to the proliferation of HUVEC cells in a HUVEC proliferation assay but not chemotactic activity in a monocyte migration assay. Other factors which were found in the high salt fraction were found to have chemotractic activity toward monocytes.

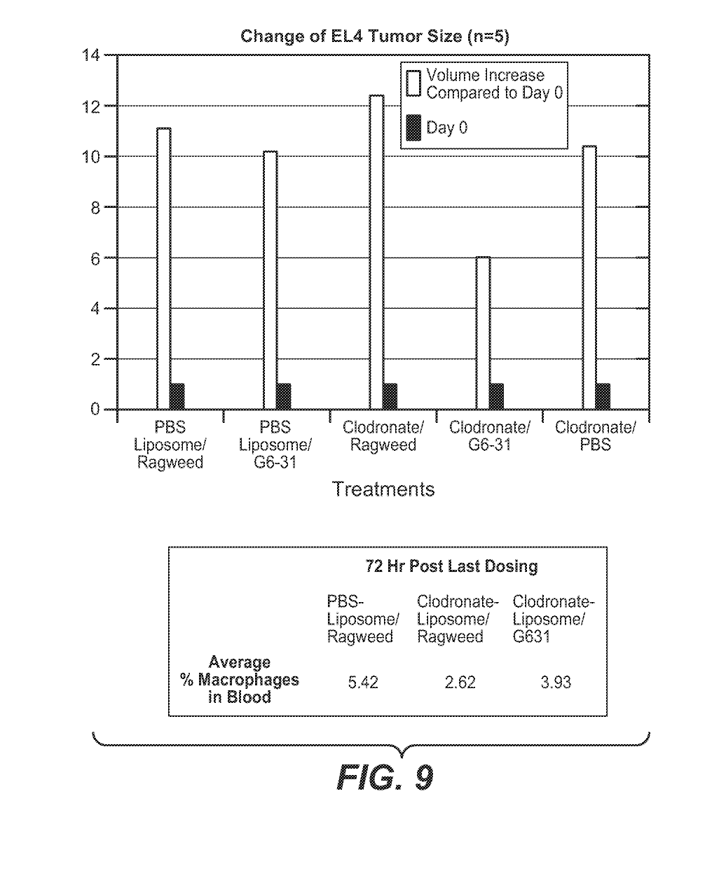

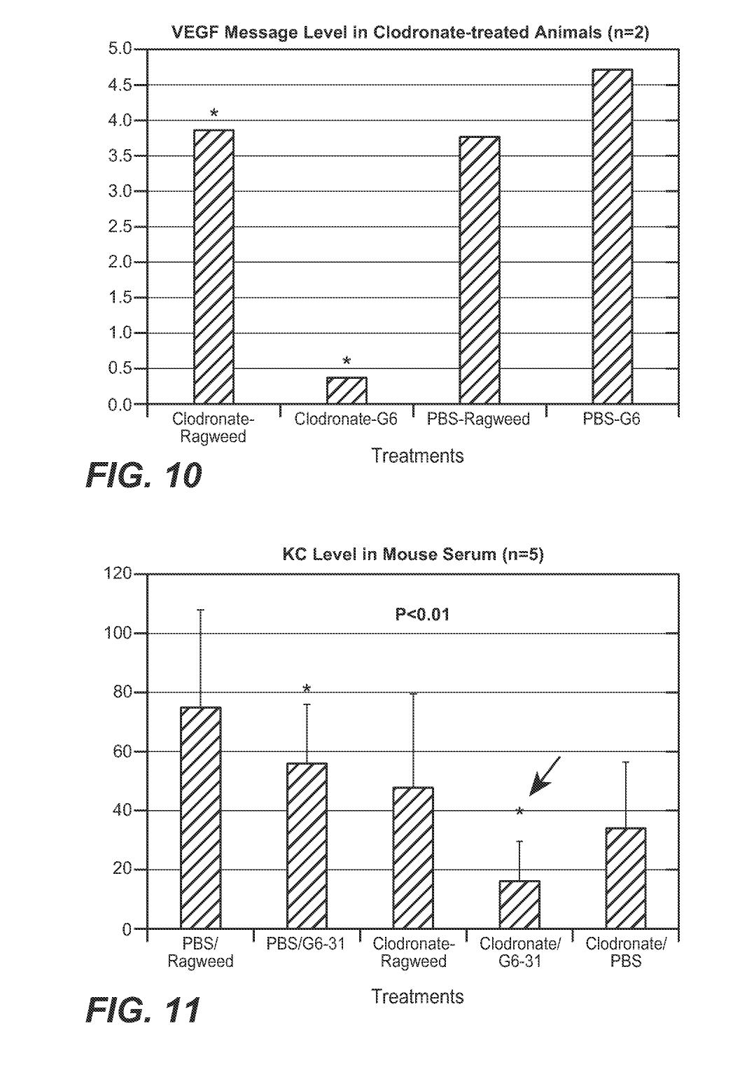

Using a combination of an agent that reduces/depletes macrophages and an anti-VEGF treatment (G6-23) in tumors (EL4) resistant to anti-VEGF treatment, the combination was found to delay tumor growth. EL4 tumors in mice were treated with 1) PBS liposome/ragweed, 2) PBS liposome/G6-31; 3) clodronate liposomes/G6-23, 4) clodronate liposomes/G6-31 or 5) clodronate liposomes/PBS in the tail vein. FIG. 9 shows the change in EL4 tumor volume (measured by caliper) 72 hours after last dosing. There is a reduction in tumor volume in mice treated with clodronate liposomes, a macrophage depleting agent, and anti-VEGF (G6-23). There was also a reduction of macrophages detected in the blood from clodronate-liposome/anti-VEGF treated animals. See bottom of FIG. 9. Clodronate liposomes also decreased VEGF expression, as measured by quantitative real-time PCR (Taqman), when administered to mice in combination with anti-VEGF (G6-23). See FIG. 10. KC (CXCL1) protein expression was also decreased, as measured by ELISA (RD Systems), in mice treated with clodronate liposomes and anti-VEGF (G6-23) as described above. See FIG. 11. KC (CXCL1) is a protein identified by its over-expression in murine monocytes and macrophages. Its synthesis is induced by TNFalpha. KC is involved in neutrophil chemotaxis/activation and arrest of rolling monocytes at endothelium surface. The synthesis of KC in vascular endothelial cells is induced by thrombin. The KC receptor and IL-8 type B receptor are homologs. The receptor is capable of binding both KC and MIP-2 (macrophage inflammatory protein-2). KC is secreted by both tumor cell lines sensitive to anti-VEGF treatment and tumor cell lines resistant to anti-VEGF treatment.

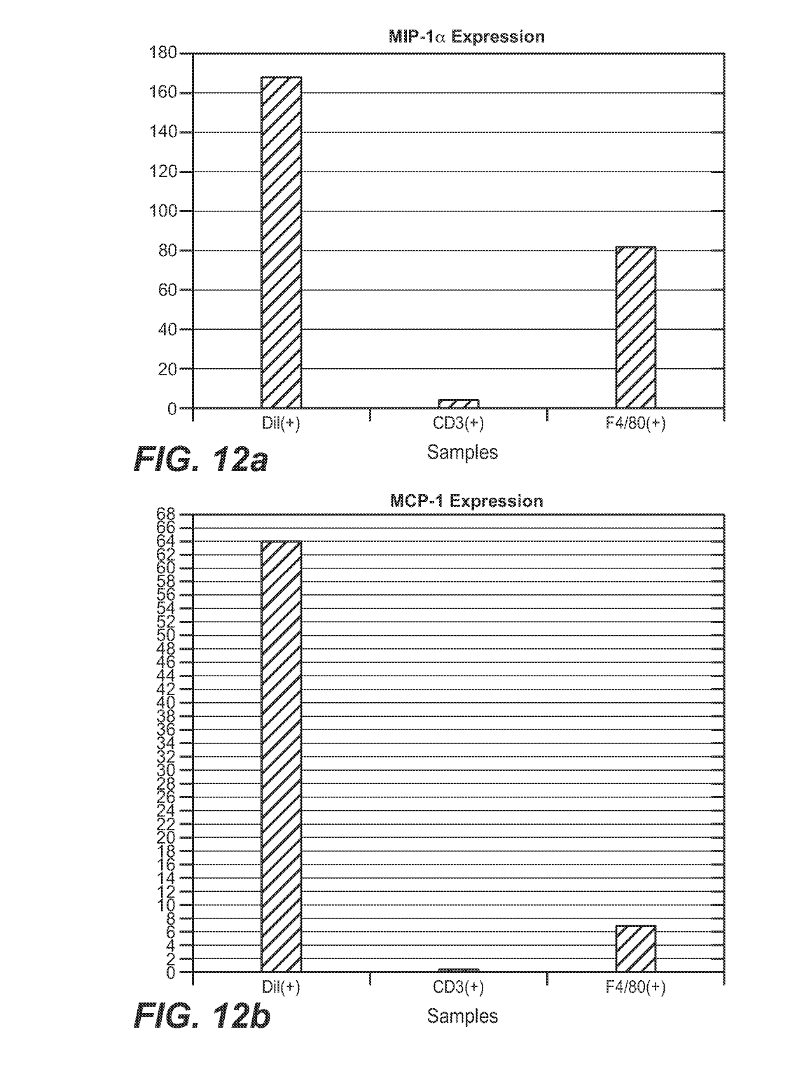

Other pro-inflammatory cytokines were found in the high salt fraction from resistant tumor cell lines, e.g., MIP-1alpha, MCP-1, IL-1alpha, IL-1beta, IL-7, IL-9, IL-10 and IL-13. MCP-1 is monocyte chemoattractant protein-1 (CCL2 or JE), and it is secreted by macrophages, fibroblasts and endothelial cells. It is induced M-CSF, IL-1, IFNgamma and TGFbeta. MIP-1alpha is macrophage inflammatory protein-1a (CCL3) it is secreted by macrophages in response to local inflammation and it activates neutrophils to produce superoxide. It is also secreted by lymphocytes and monocytes. In a mouse model of hepatocellular carcinogenesis, MIP-1alpha and MCP-1 are secreted by neovessels and stimulate proliferation through their cognate receptors in an autocrine fashion. See, e.g., Cancer Res. 66(1): 198-211 (2006). Both MCP-1 and MIP-1alpha are expressed in tumor cell lines resistant to anti-VEGF treatment. See FIG. 12, Panel A and B, where Dil(+) are endothelial cells, CD3(+) represents lymphoid cells and F4/80(+) represented macrophages. Dil stands for Dil-Ac-LDL (Acetylated Low Density lipoprotein labelled with 1,1′-dioctadecyl-3,3,3′,3′-tetramethylindo-carbocyanine perchlorate (Dil) ( Biomedical Technologies Inc). Any endothelial cells have the ability to take up this dye.

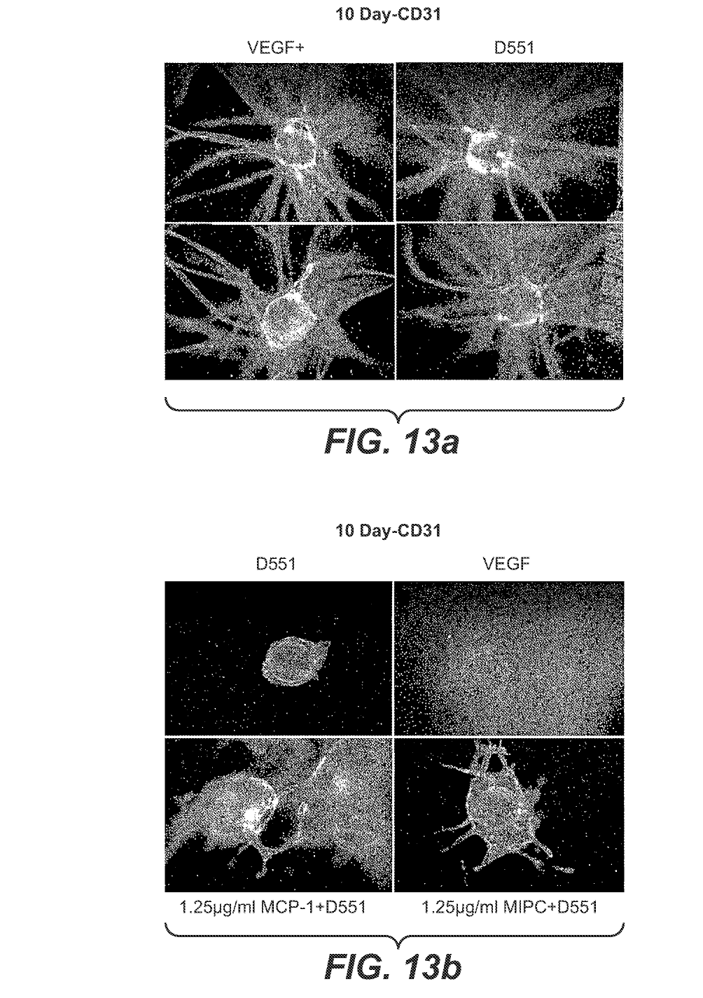

MIP-1alpha and MCP-1 were also found to have angiogenic activity in an angiogenic sprouting and capillary lumen formation assay. See FIG. 13, Panel A and Pane B on day 10. In the assays, HUVEC cells were thawed at low passage number a day before the cells were coated on beads. The cells were detached at about 80% confluency and coated on cytodex microbeads (a cross-linked dextran matrix) with HUVEC (400 cells/bead) for 4 hours at 37° C. Beads and free HUVEC cells were transferred to a flask and cultured overnight. The beads were detached and they were mixed with fibrinogen (bovine plasma) (250 μg/ml). Fibrinogen was then converted to insoluble fibrin gel by adding thrombin. There are about 100 beads/12-well plate. The beads were cultured with 40,000 D551 fibroblasts and VEGF as positive controls, D551 or VEGF as negative controls, MCP-1 and D551, or MIP-1alpha and D551. The media was changed everyday. The cultures were stained with biotinylated anti-human CD31 and Cy3 streptavidin overnight with extensive washes. Pictures were taken at 60 hours, 6 days and 10 days.

Monocyte migration assay: Step 1: isolation of monocyte from human PBMC. Blood was diluted with PBS 1:1 (v/v). The diluted blood was slowly added on top of Ficoll and centrifuged at 3000 rpm for 15 min at room temperature (RT) without break. Plasma was removed and white cells were collected (9˜5 ml interphase). Cells were washed in migration buffer containing PBS with 0.5% BSA (low in endotoxin), spun at 1850 rpm (9˜800 g) for 10 min at RT and cells were counted. Step 2: Magnetic labeling of cells. Cell pellet was resuspended in MACS buffer containing PBS with 0.5% BSA (low in endotoxin) and 2 mM EDTA, 30 μl per 10⁷ cells. FcR blocking reagent and Biotin-antibody cocktails were added and mixed well. The cells were then incubated for 10 min at 4° C. after which 30 μl more MACS buffer per 10⁷ cells was added and anti-biotin Microbeads was added. This was mixed well and incubated for 10 min at 4° C. Cells were washed with MACS buffer by adding 10-20 fold more of the labeling volume, spun at 300 g (1250 rpm) for 10 min. Cells were resuspended in up to 10⁸ cells in 2 ml buffer. Step 3: Magnetic separation with LS columns. LS column (Miltenyi Biotec) was placed in the magnetic field holder. Column was rinsed with MACS buffer. Cell suspension was applied to the column. Unlabelled flow was collected, which represents enriched monocyte fraction. Column was washed with buffer 3 times, the flow collected and combined. This was then spun at 300 g (1250 rpm) for 5 min. Step 4: Wash cells with migration media containing RPMI with 0.5% BSA (low in endotoxin) plus 2 mM L-glutamine and antibiotics. 10⁶ cells were added into 24-well Transwell plate with 5 micro meter pore size (Corning). To the outside chamber, various growth factors, cytokines/chemokines or other testing samples was added. After 2.5 hr at 37° C., the filter was carefully removed, and the cells were mixed extremely well and transfered to 10 ml ZPAK solution to count.

HUVEC proliferation assay: HUVEC at passage less than 8 were used in the study. Day 1: 3000 cells/well (96-well plate) were plated onto 1% gelatin-coated plate in the assay media (DMEM:F12 50:50) with 1.5% FBS. Day 2: Media was changed and cells were treated with various growth factors or conditioned media. Day 3: ³H-thymidine was added at 0.5 μCi/well. Day 4: 250 mM EDTA/well was added to stop the reaction in the morning. Cells were harvested onto 96-well filter plate and washed with water 3 times. ³H samples were counted with TOPCOUNT liquid scintillation counter.

In vivo treatment to examine for macrophage depletion and tumor expression: EL4, the murine lymphocyte leukemia cell line, was used. Treatment was started 48 hours after implanting EL4 tumor cells (5×10^{6,} 0.1 ml vol. in matrigel) in nude mice. Treatment was as follows: Group 1: 8 mice, twice a week PBS-liposome 200 μl IV and Ragweed IgG 2×5 mg/kg/week 100 μl ip.; Group 2: 8 mice, twice a Week: PBS-liposome 200 μl IV and G6-31 2×5 mg/kg/week 100 μl ip.; Group 3: 8 mice, twice a week: Clodronate-liposome 200 μl IV. Ragweed IgG 2×5 mg/kg/week 100 μl ip.; Group 4: 8 mice twice a week: Clodronate-liposome 200 μl IV. G6-31 2×5 mg/kg/week 100 μl ip. Group 5: 8 mice twice a week: Clodronate-liposome 200 μl Iv. PBS twice a week 100 μl ip. 3 mice of each group were pre-bled for 50 μl of whole blood for FACS macrophage cell population evaluation. 3 mice of each group were bled (optical) for 100 μl of whole blood at 1 hour after each PBS-liposome or Clodronate-liposome injection for FACS analysis. The study continued until sufficient tumor growth (not beyond 5 weeks). Tumor growth was defined as sufficient if a tumor was greater than 20 mm in length. Tumor size was measured weekly (1×w×h). Animals were observed at least twice weekly. At the endpoint of the experiment the animals were sacrificed, the tumors, measured one final time then extracted, weighed and then fixed. Blood, spleen and liver was taken from all animals for further analysis, e.g., FACS analysis, RNA analysis, etc.

Detection of Macrophage population in blood, spleen and liver as an indication of macrophage depletion: After 92 hr after first i.v. injection of clodronate liposomes, CO₂ was administrated to kill mouse (FV6 transgenic mice vs Beige Nude XID mice) (2 from Clodronate-treated FV6 and 2 from Clodronate-treated Beige Nude XID mice, one untreated Beige Nude XID mice) and 150 μl blood from heart chamber was collected and put into heparin-containing tubes and stored at RT. The blood was processed by: 1) taking 150 μl blood sample and adding 1 ml ACK Red Blood cell lysis buffer (Biosource P304-100); 2) lysing for 5 min at RT; 3) spinning 5000 rpm at RT for 2 min; 4) washing with FACS buffer (PBS+2% FCS) and spinning again; and, 5) resuspending in 60 μl FACS buffer and filtering through 70 μm mesh. The spleen was processed by: 1) preparing the single cell suspension using the frost-surface of slide glass (VWR micro slides 48312-002, 25×75mm) in FACS buffer; 2) centrifuging at 1200 rpm for 5 min; 3) suspending the pellet with 5 ml of ACK (ACK buffer: 0.15 M NH₄Cl, 10.0 mM KHCO₃, 0.1 mM Na₂ EDTA, pH to 7.2-7.4, filter sterilized through a 0.22 μm and stored at RT) and incubated at RT for 5 min or more, optionally with occasional shaking; 4) after incubation, adding FACS media to 15 ml; 5) centrifuging again and resuspending the cells in 0.5-1 ml FACS buffer (1 ml for FV6 mice, 0.5 ml for Beige nude XID mice); and 6) filtering. The liver was processed by: 1) chopping the liver (⅛ of whole piece) into small pieces in FACS buffer (on 50 ml conical tube) and washing pieces with 45 ml of PBS; 2) centrifuging the pieces at 1200 rpm for 5 min and carefully moving little pieces onto frost-surface to make single cells; 3) washing with 3 ml FACS buffer and centrifuging; and 4) resuspending in 0.5 ml FACS buffer and filter. 10 μl blood cells were diluted into 90 μl FACS buffer for total cell counting. For total cell counting, a spleen sample was taked and diluted 1:10. 50 μl samples from blood, spleen, and liver were put into 96-well cell culture cluster, V-bottom with lid (Costar 3894) and blocking antibody (CD16/32) was added at 1 μl/sample for 15 min. The cells were incubated with F4/80-PE antibody, 10 μl/sample; (Serotec, Rat anti-mouse F4/80, MCA497PE, 1101B) to detect macrophages. The cells were incubated with antibody on ice for 20 min covered with aluminum foil, after which 200 μl FACS buffer was added, and cells were centrifuge for 5 min at 4° C., 1500 rpm. The buffer was removed and the cells resuspended using 200 μl FACS buffer and were centrifuged again. Cells were finally resuspended into 130 μl FACS buffer, transfer into small tubes (202032202-12) and read on BD FACS machine.

Preparation of Single Cell Suspension from tumor sample: Tumor was dissected to remove fatty tissue and skin and put it into EL4 media containing PSGF and put on ice; tumors were washed with the same media by adding 15 ml/tumor and centrifuged at 180 rpm for 10 min; the supernatants were removed and the tissued were washed again; and, the tumors were minced into small pieces (<1 mm) into 2 ml cold EL4 media using 10 cm tissue culture dish. Single cells from the EL4 tumor were collected into a 50 ml Falcon tube by adding 8 ml media and filtered through 40 μm nylon filter mesh; 50 ml cell dissociation buffer/tumor containing collagenase IV, DNAse and elastase was added to the cells for 1.5 hr at 37° C. using 2 10 cm Petri dish. Tissue was disrupted by pipetting it up and down every 15 min. Optionally, 12.5 ml Liberase Blendyme I-containing cell dissociation buffer can be added, e.g., 200 μl into 12.5 ml after half an hour along with additional collagenase IV after 1 hr. The tissue digests were sequentially filtered through different sizes of nylon filter mesh (100, 70 and 40 μm); and samples were washed twice with EL4 media and centrifuged at 4° C. at 2000 rpm/min for 5 min. Cells were counted and collected. In one experiment, cells were lysed and total RNA was isolated (e.g., which can be analyzed by Taqman). Optionally, the cells are suspended in up to 1000000 cell/100 μl using EL4 media (1.4 ml); for 1000000 cells, cell were blocked with 2 μg FcI/II for 30 min and labeled for 1 hr at RT with F4/80, CD3 antibody or CD31 labeled antibodies to isolate macrophages, EL4 lymphocytes and other heamatopoetic cells and endothelial cells from the sample; cells were washed twice with EL4 media, suspended in 1000000 cells/0.5 ml for cell sorting. The cells are gated based on FSC/SSC and fluorescence intensity. The sorted cell can also be centrifuged, brought up into suitable culture media, counted and measured for cell viability. Cell can be prepared for morphology/immunofluorescence studies by plating cells using EL4 media on either 1% gelatin-coated or Matrigel-coated (30 min) 4-well cell culture slide chambers and cultured overnight. The cells can be loosen and lysed (<500000 cells) to isolate RNA. Optionally, the other types of cells, e.g., fibroblasts, myocytes, etc., can be isolated from sorting machine, counted and measured for cell viability, and further analysis. Optionally, these cells may be lysed and RNA isolated.

Preparation and administration of clodronate liposomes: 75-95 mg L-alpha-phosphatudylcholine was added to a 500 ml flask (that has been previously rinsed with methanol and chloroform) with 10 ml methanol and 10 ml chloroform. 10-15 mg cholesterol was added. The flask was Rotovapor with rotation (130-150 rpm) and low vacumn (gradually reducing from 200 mbar to 150 mbar) in 37° C. water bath until liquid dissolved and film formed, ˜10 min. The film was dissolved in 10 ml chloroform and placed under rotovapor again to remove chloroform and milky white phospholipids film formed around inner wall of flask. ˜15 min. In some cases, the film did not form even though liquid evaporated. The phospholipid film was dispersed in 10 ml PBS or 2.0 g clodronate/10 ml PBS and hand rotated and/or swirled until film was dissolved, in which a milky white suspension was formed. The milky white suspension was kept at RT 1.5-2 hrs under N₂ gas. The suspension was gently shook and sonicated in in waterbath sonicator for 3 min. The suspension was kept under N₂ gas for 2 hrs RT or overnight 4° C. for liposome swelling. Non-encapsulated clodronate was removed by centrifugation of liposomes 10,000×g, 15 min, 16° C. (11,600 rpm 70 Ti rotor). The liposomes formed a white band at top of suspension. Clodronate solution underneath liposomes was removed using a pipette. The liposomes were washed 2-3 times with sterile PBS and swirled by hand to disrupt pellet. The liposomes were spun 25,000×g, 30 min, 16° C. (18,400 rpm using 70 Ti rotor). The pellet was resuspended in 4 ml sterile PBS and stored up to 4 weeks under N₂ in PBS up to 4 weeks. Before administering to animals, the liposomes were gently shaken and 200 μl liposome reagent was administered to each animals via tail vein, twice every week.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

We claim:

1. A method of treating a resistant tumor in a subject with a combination treatment, the method comprising:
administering an effective amount of a VEGF antagonist in combination with an effective amount of a second agent to the subject with the resistant tumor, wherein the second agent comprises a myeloid cell reduction agent.

2. The method of claim 1, wherein the myeloid cell reduction agent comprises a Gr1 antagonist, an elastase inhibitor, a MCP-1 antagonist, or a MIP-1 alpha antagonist.

3. The method of claim 2, wherein the antagonist is an antibody.

4. A method of diagnosising a resistant tumor in a subject, the method comprising:
providing from the subject a test cell population from a tumor of the subject;
measuring the number or percentage of CD11b+Gr1+ cells in the test cell population;
comparing the number or percentage of the CD11b+Gr1+ cells in the test cell population to the number or percentage of the CD11b+Gr1+ cells in a reference cell population; and,
detecting an increase in the number or percentage of CD11b+Gr1+ cells in the test cell population compared to reference cell population, wherein the increase in number or percentage of CD11b+Gr1+ cells indicates that the tumor is the resistant tumor.

5. The method of claim 4, further comprising measuring spleen size of the subject and comparing the spleen size of the subject to a reference spleen size, wherein enlarged spleen size indicates that the tumor is the resistant tumor.

6. The method of claim 4, further comprising measuring the number or percentage of a vascular surface area (VSA) of a tumor in the subject after the subject has been administered a VEGF antagonist, and comparing the number or percentage of the vascular surface area number of the tumor in the subject to a reference vascular surface area, wherein an increase in the number or percentage of the vascular surface area of the tumor indicates that the tumor is the resistant tumor.

7. The method of claim 6 wherein the antagonist is an antibody.

8. The method of claim 4, further comprising:
providing from the subject a test cell population from a tumor of the subject;
measuring the number or percentage of CD19 B-lymphoid cells or CD11c dendritic cells in the test cell population;
comparing the number or percentage of the CD19 B-lymphoid cells or CD11c dendritic cells in the test cell population to the number or percentage of the CD19 B-lymphoid cells or CD11c dendritic cells in a reference cell population; and,
detecting a decrease in the number or percentage of CD19 B-lymphoid cells or CD11c dendritic cells in the test cell population compared to reference cell population, wherein the decrease in number or percentage of CD19 B-lymphoid cells or CD11c dendritic cells indicates that the tumor is the resistant tumor.

9. The method of claim 4, further comprising:
providing from the subject a test cell population from a bone marrow of the subject; measuring the number or percentage of CD90 T-lymphoid cells, CD19 B-lymphoid cells or CD11c dendritic cells in the test cell population; comparing the number or percentage of the CD90 T-lymphoid cells, CD19 B-lymphoid cells or CD11c dendritic cells in the test cell population to the number or percentage of the CD90 T-lymphoid cells, CD19 B-lymphoid cells or CD11c dendritic cells in a reference cell population; and, detecting a decrease in the number or percentage of CD90 T-lymphoid cells, CD19 B-lymphoid cells or CD11c dendritic cells in the test cell population compared to reference cell population, wherein the decrease in number or percentage of CD90 T-lymphoid cells, CD19 B-lymphoid cells or CD11c dendritic cells indicates that the tumor is the resistant tumor.

10. A method of treating a resistant tumor in a subject with a combination treatment, the method comprising:
administering an effective amount of a VEGF antagonist in combination with an effective amount of a myeloid cell reduction agent and an effective amount of a third agent to the subject with the resistant tumor, wherein the third agent is a chemotherapeutic agent.

11. The method of claim 10 wherein the antagonist is an antibody.

12. The method of claim 10 wherein the myeloid cell reduction agent comprises a Gr1 antagonist, an elastase inhibitor, a MCP-1 antagonist, or a MIP-1 alpha antagonist.

13. The method of claim 10 wherein the chemotherapeutic agent is 5FU or gemcitabine.
